(19) United States
(12) Patent Application Publication
(10) Pub. No.: US 2020/0190585 A1
(43) Pub. Date: Jun. 18, 2020

(54) Data Processing and Classification for Determining a Likelihood Score for Immune-Related Adverse Events

(71) Applicant: Checkpoint Sciences, Inc., Dover, MA (US)

(72) Inventor: Karl Wassmann, Dover, MA (US)

(73) Assignee: Checkpoint Sciences, Inc., Dover, MA (US)

(21) Appl. No.: 16/486,086

(22) PCT Filed: Feb. 14, 2018
(86) PCT No.: PCT/US2018/018214

§ 371 Date: Aug. 14, 2019

Related U.S. Application Data
(60) Provisional application No. 62/459,489, filed on Feb. 15, 2017.

(51) Int. Cl. C12Q 1/6883; G16B 20/00; G16H 50/20; G16H 10/40; G16H 10/20; G16H 20/10; G06N 5/04; G06N 20/00

(52) U.S. Cl. CPC C12Q 1/6883; G16B 20/00; G16H 50/20; G16H 10/40; C12Q 2600/158; G16H 20/10; G06N 5/04; G06N 20/00; C12Q 2600/106; G16H 10/20

(57) ABSTRACT

The present disclosure relates to a computer-implemented method for processing data in one or more data processing devices to determine a likelihood score or the probability of immune-related adverse events associated with immunotherapy.

### CLAIM OF PRIORITY

This application claims the benefit of U.S. Provisional Application Ser. No. 62/459,489, filed on Feb. 15, 2017, the entire contents of which are incorporated herein by reference.

### FIELD

The disclosure relates to data processing methods, computer readable hardware storage devices, and systems for correlating data corresponding to levels of biomarkers with immune-related adverse events associated with immunotherapy.

### BACKGROUND

A classifier maps input data to a category, by determining the probability that the input data classifies with a first category as opposed to another category. There are various types of classifiers, including linear discriminant classifiers, logistic regression classifiers, support vector machine classifiers, nearest neighbor classifiers, ensemble classifiers, and so forth.

### SUMMARY

The present disclosure relates to a computer-implemented method for processing data in one or more data processing devices to determine the likelihood score for, or the probability of, immune-related adverse events associated with immunotherapy.

In one aspect, the disclosure relates to computer-implemented methods for processing data in one or more data processing devices to determine a likelihood score for an immune-related adverse event associated with an immunotherapy given to a test subject. The methods include the steps of:

inputting, into a classifier, data representing one or more values for a classifier parameter that represents a gene-specific level of mRNA transcribed from a gene of a set of genes in a sample of blood collected from a test subject who was treated with the immunotherapy prior to collecting the sample, with the input data specifying a gene-specific level of mRNA transcribed from each gene of the set of genes in the sample of blood of the test subject, the set of genes comprising CCR3 and PTGS2, with the classifier being for determining a likelihood score indicating whether the gene-specific levels of mRNA transcribed from each gene in the set of genes classifies with (A) a set of immunotherapy-intolerance levels, the set of immunotherapy-intolerance levels being a set of gene-specific levels of mRNA transcribed from each gene of the set of genes in blood samples collected from a first group of individuals who were treated with the immunotherapy prior to collecting the sample, wherein each individual of the first group experienced the immune-related adverse event associated with the immunotherapy; as opposed to classifying with (B) a set of immunotherapy-tolerance levels, the set of immunotherapy-tolerance levels being a set of gene-specific levels of mRNA transcribed from each gene of the set of genes in blood samples collected from a second group of individuals who were treated with the immunotherapy prior to collecting the sample, wherein each individual of the second group did not experience the immune-related adverse event associated with the immunotherapy;

for each of one or more of the genes in the set, binding, by the one or more data processing devices, to the classifier parameter one or more values representing a gene-specific level of transcribed mRNA from that gene as specified by the input data;

applying, by the one or more data processing devices, the classifier to bound values for the parameter;

determining, by the one or more data processing devices based on application of the classifier, the likelihood score for the immune-related adverse event for the test subject; and

outputting, by the one or more data processing devices, information indicative of the determined likelihood score for the immune-related adverse event for the test subject.

In another aspect, the disclosure provides one or more machine-readable hardware storage devices for processing data to determine a likelihood score for an immune-related adverse event associated with an immunotherapy given to a test subject by storing instructions that are executable by one or more data processing devices to perform operations comprising:

inputting, into a classifier, data representing one or more values for a classifier parameter that represents a gene-specific level of mRNA transcribed from a gene of a set of genes in a sample of blood collected from a test subject who was treated with the immunotherapy prior to collecting the sample, with the input data specifying a gene-specific level of mRNA transcribed from each gene of the set of genes in the sample of blood of the test subject, the set of genes comprising CCR3 and PTGS2, with the classifier being for determining a likelihood score indicating whether the gene-specific levels of mRNA transcribed from each gene in the set of genes classifies with (A) a set of immunotherapy-intolerance levels, the set of immunotherapy-intolerance levels being a set of gene-specific levels of mRNA transcribed from each gene of the set of genes in blood samples collected from a first group of individuals who were treated with the immunotherapy prior to collecting the sample, wherein each individual of the first group experienced the immune-related adverse event associated with the immunotherapy; as opposed to classifying with (B) a set of immunotherapy-tolerance levels, the set of immunotherapy-tolerance levels being a set of gene-specific levels of mRNA transcribed from each gene of the set of genes in blood samples collected from a second group of individuals who were treated with the immunotherapy prior to collecting the sample, wherein each individual of the second group did not experience the immune-related adverse event associated with the immunotherapy;

for each of one or more of the genes in the set, binding, by the one or more data processing devices, to the classifier parameter one or more values representing a gene-specific level of transcribed mRNA from that gene as specified by the input data;

applying, by the one or more data processing devices, the classifier to bound values for the parameter;

determining, by the one or more data processing devices based on application of the classifier, the likelihood score for the immune-related adverse event for the test subject; and

outputting, by the one or more data processing devices, information indicative of the determined likelihood score for the immune-related adverse event for the test subject.

The disclosure also provides systems comprising:

one or more data processing devices; and

one or more machine-readable hardware storage devices for processing data to determine a likelihood score for an immune-related adverse event associated with an immunotherapy given to a test subject by storing instructions that are executable by one or more data processing devices to perform operations comprising:

inputting, into a classifier, data representing one or more values for a classifier parameter that represents a gene-specific level of mRNA transcribed from a gene of a set of genes in a sample of blood collected from a test subject who was treated with the immunotherapy prior to collecting the sample, with the input data specifying a gene-specific level of mRNA transcribed from each gene of the set of genes in the sample of blood of the test subject, the set of genes comprising CCR3 and PTGS2, with the classifier being for determining a likelihood score indicating whether the gene-specific levels of mRNA transcribed from each gene in the set of genes classifies with (A) a set of immunotherapy-intolerance levels, the set of immunotherapy-intolerance levels being a set of gene-specific levels of mRNA transcribed from each gene of the set of genes in blood samples collected from a first group of individuals who were treated with the immunotherapy prior to collecting the sample, wherein each individual of the first group experienced the immune-related adverse event associated with the immunotherapy; as opposed to classifying with (B) a set of immunotherapy-tolerance levels, the set of immunotherapy-tolerance levels being a set of gene-specific levels of mRNA transcribed from each gene of the set of genes in blood samples collected from a second group of individuals who were treated with the immunotherapy prior to collecting the sample, wherein each individual of the second group did not experience the immune-related adverse event associated with the immunotherapy;

for each of one or more of the genes in the set, binding, by the one or more data processing devices, to the classifier parameter one or more values representing a gene-specific level of transcribed mRNA from that gene as specified by the input data;

applying, by the one or more data processing devices, the classifier to bound values for the parameter;

determining, by the one or more data processing devices based on application of the classifier, the likelihood score for the immune-related adverse event for the test subject; and

outputting, by the one or more data processing devices, information indicative of the determined likelihood score for the immune-related adverse event for the test subject.

In some embodiments, the input data comprise one or more records that each have one or more values for the parameter representing the level of transcribed mRNA; and wherein determining the likelihood score for the immune-related adverse event for the test subject comprises: determining, by the one or more data processing devices based on application of the classifier to the input data comprising the one or more records, the likelihood score for the immune-related adverse event for the test subject.

In one aspect, the disclosure also relates to methods comprising:

a) obtaining a biological sample from a subject who is undergoing immunotherapy;

b) determining, from the biological sample, gene-specific levels of mRNA transcribed from each gene of a set of genes, wherein the set of genes comprises CCR3 and PTGS2,

c) determining that the gene-specific levels of mRNA transcribed from each gene in the set of genes are classified with (A) a set of immunotherapy-intolerance levels, the set of immunotherapy-intolerance levels being a set of gene-specific levels of mRNA transcribed from each gene of the set of genes in blood samples collected from a first group of individuals who were treated with the immunotherapy prior to collecting the sample, wherein each individual of the first group experienced an immune-related adverse event associated with the immunotherapy; rather than being classified with (B) a set of immunotherapy-tolerance levels, the set of immunotherapy-tolerance levels being a set of gene-specific levels of mRNA transcribed from each gene of the set of genes in blood samples collected from a second group of individuals who were treated with the immunotherapy prior to collecting the sample, wherein each individual of the second group did not experience the immune-related adverse event associated with the immunotherapy;

d) either (i) providing to the subject a degree of monitoring for early symptoms related to development of the immune-related adverse event that is heightened compared to the degree of monitoring (if any) for the early symptoms provided to the subject between the start of the immunotherapy and the time the determination of (c) was made; or (ii) administering to the subject a treatment suitable for reducing the likelihood the subject will actually experience the immune-related adverse event; or (iii) reducing the subject's dosage of the immunotherapy; or (iv) a combination of any two or more of (i), (ii), and (iii).

In some embodiments, the immune-related adverse event is Grade 3 diarrhea, Grade 4 diarrhea, or colitis. In some embodiments, the second group of individuals did not experience diarrhea, or experienced diarrhea no more severe than Grade 1 or Grade 2 diarrhea. In some embodiments, the set of genes comprises CCR3, MMP9, and PTGS2. In some embodiments, the set of genes further comprises at least one, at least two, at least three, at least four, or all genes selected from the group consisting of CARD12, CCND1, IL5, F5 and GYPA.

In some embodiments, the immune-related adverse event is Grade 2, Grade 3, or Grade 4 diarrhea, or colitis. In some embodiments, the second group of individuals did not experience diarrhea, or experienced diarrhea no more severe than Grade 1 diarrhea. In some embodiments, the set of genes comprises CCL3, CCR3, IL8, and PTGS2. The set of genes can further comprise at least one, at least two, at least three, at least four, at least five, or all genes selected from the group consisting of CARD12, F5, MMP9, SOCS3, IL5, and TLR9.

In some embodiments, the set of genes further comprises at least one gene, at least two genes, at least three genes, at least four genes, at least five genes, at least six genes, at least seven genes, at least eight genes, at least nine genes, at least ten genes, at least eleven genes, at least twelve genes, at least thirteen genes, at least fourteen genes, at least fifteen genes, or all sixteen genes selected from the group consisting of CARD12, CDC25A, CXCL1, F5, FAM210, GADD45A, IL18BP, IL2RA, IL5, IRAK3, ITGA4, MAPK14, MMP9, SOCS3, TLR9, and UBE2C.

In some embodiments, the classifier has a form:

Y=α+ΣβᵢXᵢ

wherein

Y is a likelihood score indicating a probability that the set of test levels classifies with the set of immunotherapy-intolerance levels, as opposed to the set of immunotherapy-tolerance levels,

Xᵢ is a level of mRNA transcribed from an ith gene of the set of genes in blood of the test subject,

βᵢ is a logistic regression equation coefficient for the ith gene,

α is a logistic regression equation constant that can be zero, and

βᵢ and α are the result of applying logistic regression analysis to the set of immunotherapy-intolerance levels and the set of immunotherapy-tolerance levels.

The disclosure also provides computer-implemented methods for processing data in one or more data processing devices to determine a likelihood score for developing Grade 2, Grade 3, or Grade 4 diarrhea in a test subject receiving an immunotherapy. The methods include the steps of:

inputting, into a classifier, data representing one or more values for a classifier parameter that represents a gene-specific level of mRNA transcribed from a gene of a set of genes in a sample of blood collected from a test subject who was treated with the immunotherapy prior to collecting the sample, with the input data specifying a gene-specific level of mRNA transcribed from each gene of the set of genes in the sample of blood of the test subject, the set of genes comprising CCR3 and PTGS2, with the classifier being for determining a likelihood score indicating whether the gene-specific levels of mRNA transcribed from each gene in the set of genes classifies with (A) a set of immunotherapy-intolerance levels, the set of immunotherapy-intolerance levels being a set of gene-specific levels of mRNA transcribed from each gene of the set of genes in blood samples collected from a first group of individuals who were treated with the immunotherapy prior to collecting the sample, wherein each individual of the first group experienced Grade 2, Grade 3, or Grade 4 diarrhea; as opposed to classifying with (B) a set of immunotherapy-tolerance levels, the set of immunotherapy-tolerance levels being a set of gene-specific levels of mRNA transcribed from each gene of the set of genes in blood samples collected from a second group of individuals who were treated with the immunotherapy prior to collecting the sample, wherein each individual of the second group experienced Grade 1 diarrhea but did not experience a higher grade of diarrhea;

for each of one or more of the genes in the set, binding, by the one or more data processing devices, to the classifier parameter one or more values representing a gene-specific level of transcribed mRNA from that gene as specified by the input data;

applying, by the one or more data processing devices, the classifier to bound values for the parameter;

determining, by the one or more data processing devices based on application of the classifier, the likelihood score for developing Grade 2, Grade 3, or Grade 4 diarrhea in the test subject; and

outputting, by the one or more data processing devices, information indicative of the determined likelihood score for developing Grade 2, Grade 3, or Grade 4 diarrhea in the test subject.

The disclosure also provides methods comprising:

a) obtaining a biological sample from a subject who is undergoing immunotherapy and is identified as having one or more diarrhea symptoms;

b) determining, from the biological sample, gene-specific levels of mRNA transcribed from each gene of a set of genes, wherein the set of genes comprises CCR3 and PTGS2,

c) determining that the gene-specific levels of mRNA transcribed from each gene in the set of genes are classified with (A) a set of immunotherapy-intolerance levels, the set of immunotherapy-intolerance levels being a set of gene-specific levels of mRNA transcribed from each gene of the set of genes in blood samples collected from a first group of individuals who were treated with the immunotherapy prior to collecting the sample, wherein each individual of the first group experienced Grade 2, Grade 3, or Grade 4 diarrhea at some point during that individual's immunotherapy treatment period; rather than being classified with (B) a set of immunotherapy-tolerance levels, the set of immunotherapy-tolerance levels being a set of gene-specific levels of mRNA transcribed from each gene of the set of genes in blood samples collected from a second group of individuals who were treated with the immunotherapy prior to collecting the sample, wherein each individual of the second group experienced Grade 1 diarrhea at some point during that individual's immunotherapy treatment period but did not experience a higher grade of diarrhea during that period; and

d) administering an anti-inflammatory agent to the subject.

In some embodiments, the set of genes comprises CCL3, CCR3, IL8, and PTGS2. The set of genes can further comprise at least one, at least two, at least three, at least four, at least five, or all genes selected from the group consisting of CARD12, F5, MMP9, SOCS3, IL5, and TLR9.

In some embodiments, the set of genes further comprises at least one gene, at least two genes, at least three genes, at least four genes, at least five genes, at least six genes, at least seven genes, at least eight genes, at least nine genes, at least ten genes, at least eleven genes, at least twelve genes, at least thirteen genes, at least fourteen genes, at least fifteen genes, or all sixteen genes selected from the group consisting of CARD12, CDC25A, CXCL1, F5, FAM210, GADD45A, IL18BP, IL2RA, IL5, IRAK3, ITGA4, MAPK14, MMP9, SOCS3, TLR9, and UBE2C.

As used herein, a “gene” refers to a locus (or segment) of DNA that is transcribed into a functional RNA product or encodes a functional protein or peptide product.

As used herein, “a set of” refers to two or more, for example, 2, 3, 4, 5, 6, 7, 8, 9, 10, or more.

As used herein, a “blood sample” or “sample of blood” refers to whole blood, serum-reduced whole blood, lysed blood (erythrocyte-depleted blood), centrifuged lysed blood (serum-depleted, erythrocyte-depleted blood), serum-depleted whole blood or peripheral blood leukocytes (PBLs), globin-reduced RNA from blood, or any other fraction of blood as would be understood by a person skilled in the art.

As used herein, “immunotherapy” refers to a type of cancer treatment designed to alter the body's natural immunological defenses to fight the cancer. Immunotherapy can induce, enhance, or suppress an immune response. Immunotherapy can be, for example, an interferon, an interleukin, or an antibody that targets receptors or ligands that are involved in the immune system. Current antibody immunotherapies include, but are not limited to, alemtuzumab, ipilimumab, ofatumumab, nivolumab, pembrolizumab, rituximab, and so forth. Antibody immunotherapies are described in detail in Creelan, Benjamin C., “Update on immune checkpoint inhibitors in lung cancer,” Cancer Control 21.1 (2014): 80-89, which is incorporated by reference in its entirety.

As used herein, “mRNA” refers to an RNA complementary to the exons of a gene. An mRNA sequence includes a protein coding region or part of the coding region, and also may include 5′ and 3′ untranslated regions (UTR).

As used herein, each of “patient,” “individual,” and “subject” refers to a mammal, which in some embodiments is a human.

As used herein, “level” or “level of expression,” when referring to RNA, means a measurable quantity (either absolute or relative quantity) of a given mRNA. The quantity can be determined by various means, for example, by microarray, quantitative polymerase chain reaction (QPCR), or sequencing.

As used herein, a “primer” refers to an oligonucleotide that is capable of acting as a point of initiation of DNA or RNA synthesis complementary to a strand of nucleic acid, when placed under conditions in which synthesis of a primer extension product complementary to the nucleic acid strand is induced, i.e., in the presence of mononucleotides and an inducing agent such as a DNA polymerase and at a suitable temperature and pH. In some embodiments, the primer may be single-stranded and is sufficiently long to prime the synthesis of the desired extension product in the presence of the inducing agent.

As used herein, “cancer” refers to cells having the capacity for autonomous growth within an animal. Examples of such cells include cells having an abnormal state or condition characterized by rapidly proliferating cell growth. Cancer further includes cancerous growths, e.g., tumors, oncogenic processes, metastatic tissues, and malignantly transformed cells, tissues, or organs, irrespective of histopathologic type or stage of invasiveness. Cancer further includes malignancies of the various organ systems, such as skin, respiratory, cardiovascular, renal, reproductive, hematological, neurological, hepatic, gastrointestinal, and endocrine systems; as well as adenocarcinomas, which include malignancies such as most colon cancers, renal-cell carcinoma, prostate cancer, testicular tumors, non-small cell carcinoma of the lung, cancer of the small intestine, and cancer of the esophagus. Cancer that is “naturally arising” includes any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections. The methods described herein can determine the likelihood score for, or the probability of, an adverse reaction to immunotherapy treatment (e.g., diarrhea) for various cancers, including cancers of the skin (e.g., melanoma, unresectable melanoma, or metastatic melanoma), stomach, colon, rectum, mouth/pharynx, esophagus, larynx, liver, pancreas, lung, breast, cervix uteri, corpus uteri, ovary, prostate, testis, bladder, bone, kidney, head, neck, brain/central nervous system, and throat etc., and also Hodgkins disease, non-Hodgkins lymphoma, sarcomas, choriocarcinoma, lymphoma, neuroblastoma (e.g., pediatric neuroblastoma), chronic lymphocytic leukemia, and squamous non-small cell lung cancer, among others.

As used herein, “melanoma” refers to a type of skin cancer that develops from melanocytes, the skin cells in the epidermis that produce the skin pigment melanin. As used herein, melanoma includes Stage I, Stage II, Stage III and Stage IV melanoma, as determined by the American Joint Committee on Cancer (AJCC) (6th Edition), non-melanotic melanoma, nodular melanoma, acral lentiginous melanoma, and lentigo maligna. “Active melanoma” is a type of melanoma in which subjects have clinical evidence of disease. “Inactive melanoma” includes melanoma in which subjects have no clinical evidence of disease.

As used herein, “prostate cancer” refers to cancer in the prostate gland. Castration-resistant prostate cancer is a subcategory of prostate cancer that is not responsive to castration treatment (reduction of available androgen/testosterone/DHT by chemical or surgical means).

As used herein, “colon cancer” refers to cancer in the colon or rectum.

As used herein, a “biomarker” refers to a measurable indicator of some biological state or condition, for example, a particular mRNA or protein, or a particular combination of mRNAs or proteins.

As used herein, the term “data” in relation to biomarkers generally refers to data reflective of the absolute and/or relative abundance (level) of a biomarker in a sample, for example, the level of one or more particular transcribed mRNAs, or the amount of one or more particular proteins. As used herein, a “dataset” in relation to biomarkers refers to a set of data representing the absolute and/or relative abundance (level) of one biomarker or a panel of two or more biomarkers in a group of subjects.

As used herein, a “mathematical model” refers to a description of a system using mathematical concepts and language. The process of developing a mathematical model is termed mathematical modeling or model construction.

As used herein, the term “classifier” refers to a mathematical model with appropriate parameters that can determine a likelihood score or a probability that a test subject classifies with a first group of subjects (e.g., a group of subjects that experienced immune-related adverse events following treatment with an immunotherapy) as opposed to another group of subjects (e.g., a group of subjects that does not experience immune-related adverse events after such treatment).

As used herein, the terms “immune-related adverse events” and “adverse reactions to an immunotherapy” respectively refer to adverse events associated with an immunotherapy treatment or undesirable reactions associated with an immunotherapy treatment.

As used herein, the term “immunotherapy-induced diarrhea” refers to diarrhea directly or indirectly caused by immunotherapy. Toxicity levels of diarrhea are typically categorized into Grades 1-4. Grade 1 refers to mild diarrhea, Grade 2 refers to moderate diarrhea, Grade 3 refers to severe diarrhea, and Grade 4 refers to potentially life-threatening diarrhea (See Food and Drug Administration, “Toxicity grading scale for healthy adult and adolescent volunteers enrolled in preventive vaccine clinical trials,” US Department of Health and Human Services (2007)). The category of “Grade 0” diarrhea is used to denote that the subject does not have observable diarrhea.

As used herein, the term “colitis” refers to the inflammation of the colon. The term “immunotherapy-induced colitis” refers to colitis directly or indirectly caused by immunotherapy.

As used herein, “random selection” or “randomly selected” refers to a method of selecting items (often called units) from a group of items or a population randomly. The probability of choosing a specific item is the proportion of those items in the population. For example, the probability of randomly selecting one particular gene out of a group of 10 genes is 0.1.

Unless otherwise defined, technical and scientific terms used herein have the same meaning as commonly understood by one of ordinary skill in the art. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. The materials, methods, and examples provided here are illustrative only and not intended to be limiting.

Other features and advantages of the methods described herein will be apparent from the following detailed description, and from the claims.

### BRIEF DESCRIPTION OF THE DRAWINGS

FIG. 1 is a schematic diagram showing a system for processing and classifying data to determine a likelihood score for immune-related adverse events associated with immunotherapy.

FIG. 2 is a flow diagram of a process for processing and classifying data to determine a likelihood score for immune-related adverse events associated with immunotherapy.

### DETAILED DESCRIPTION

This disclosure relates to a computer-implemented method for processing data to determine a likelihood score for immune-related adverse events associated with immunotherapy. A data processing system consistent with this disclosure applies classifiers to data corresponding to levels of transcribed mRNAs of a set of genes.

The practice of the present disclosure will also partly employ, unless otherwise indicated, techniques of molecular biology, microbiology and recombinant DNA that are familiar to one of skilled in the art. Such techniques are explained fully in the literature. See, e.g., Molecular Cloning: A Laboratory Manual (Michael R. Green, Joseph Sambrook, Fourth Edition, 2012); Oligonucleotide Synthesis: Methods and Applications (Methods in Molecular Biology) (Piet Herdewijn, 2004); Nucleic Acid Hybridization (M. L. M. Andersen, 1999); Short Protocols in Molecular Biology (Ausubel et al., 1990).

### Data Processing System

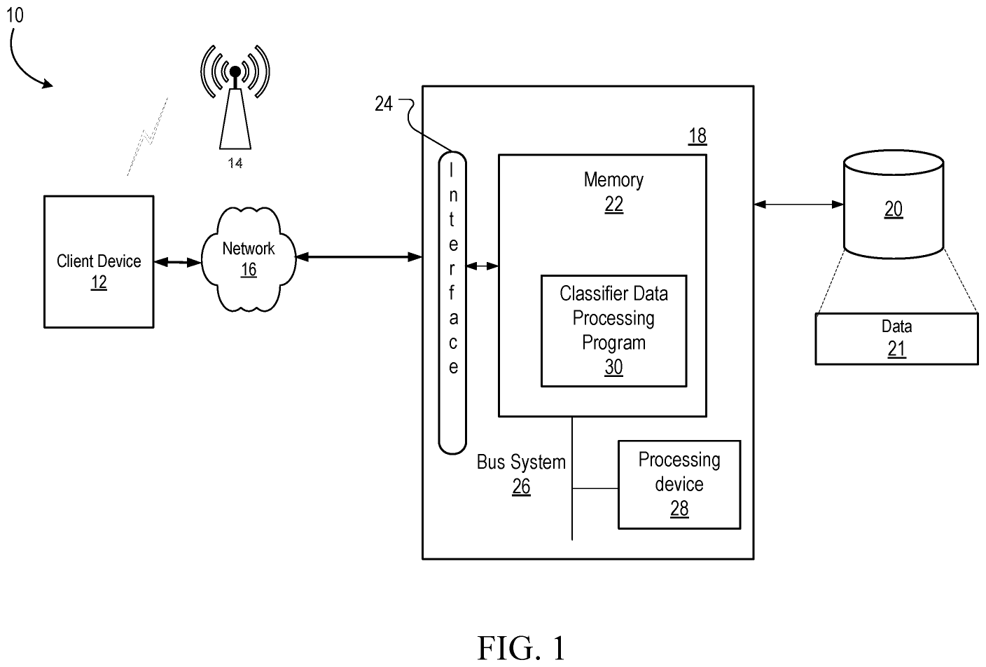

Referring to FIG. 1, system 10 classifies groups of data via binding data to parameters and applying a classifier to the input data, and outputs information indicative of a likelihood score for an immune-related adverse event associated with an immunotherapy. System 10 includes client device 12, data processing system 18, data repository 20, network 16, and wireless device 14.

Data processing system 18 retrieves, from data repository 20, data 21 representing one or more values for a classifier parameter that represents a gene-specific level of transcribed mRNA from a gene of a set of genes in a sample of blood of a test subject, as described in further detail below. Data processing system 18 inputs the retrieved data into a classifier, e.g., into classifier data processing program 30. In this embodiment, classifier data processing program 30 is programmed to execute a data classifier. There are various types of data classifiers, including, e.g., linear discriminant classifiers, support vector machine classifiers, nearest neighbor classifiers, ensemble classifiers, and so forth. In this embodiment, classifier data processing program 30 is configured to execute a classifier in accordance with the below equation:

Y=α+ΣβᵢXᵢ

In this embodiment, Y is a likelihood score indicating the probability that the set of test levels classifies with a set of immunotherapy-intolerance levels, as opposed to a set of immunotherapy-tolerance levels. The set of immunotherapy-intolerance levels is a set of gene-specific levels of mRNA transcribed from each gene of the set of genes in blood samples collected from a first group of individuals who were treated with the immunotherapy prior to collecting the sample, wherein each individual of the first group experienced the immune-related adverse event following the treatment (either before or after the blood sample was collected). The set of immunotherapy-tolerance levels is a set of gene-specific levels of mRNA transcribed from each gene of the set of genes in blood samples collected from a second group of individuals who were treated with the immunotherapy prior to collecting the sample, wherein each individual of the second group did not experience the immune-related adverse event following the treatment, whether before or after the individual's blood sample was collected.

Xᵢ is a level of mRNA transcribed from an ith gene of the set of genes in blood of the test subject. βᵢ is a logistic regression equation coefficient for the ith gene. α is a logistic regression equation constant that can be zero. βᵢ and α are the result of applying logistic regression analysis to the set of immunotherapy-intolerance levels and the set of immunotherapy-tolerance levels.

In this embodiment, Xᵢ represents a classifier parameter. Data processing system 18 binds to classifier parameter Xᵢ one or more values representing a gene-specific level of transcribed mRNA from that gene, as specified in retrieved data 21. Data processing system 18 binds values of the data to the classifier parameter by modifying a database record such that a value of the parameter is set to be the value of data 21 (or a portion thereof). Data 21 includes a plurality of data records that each have one or more values for the parameter Xᵢ representing the level of transcribed mRNA, and in some embodiments, some parameters of the classifier (e.g., values for logistic regression equation coefficients and logistic regression equation constants). In one embodiment, data processing system 18 applies classifier data processing program 30 to each of the records by applying classifier data processing program 30 to the bound values for the parameter Xᵢ. Based on application of classifier data processing program 30 to the bound values (e.g., as specified in data 21 or in records in data 21), data processing system 18 determines a likelihood score indicating a probability that the set of test levels classifies with the set of immunotherapy-intolerance levels, as opposed to the set of immunotherapy-tolerance levels, and outputs, e.g., to client device 12 via network 16 and/or wireless device 14, data indicative of the determined likelihood score for the immune-related adverse event for the test subject.

Data processing system 18 generates data for a graphical user interface that, when rendered on a display device of client device 12, display a visual representation of the output. In one embodiment, data processing system 18 generates the classifier by applying the mathematical model to a dataset to determine parameters of a classifier (e.g., values for logistic regression equation coefficients and logistic regression equation constants). The values for these parameters can be stored in data repository 20 or memory 22.

Client device 12 can be any sort of computing device capable of taking input from a user and communicating over network 16 with data processing system 18 and/or with other client devices. Client device 12 can be a mobile device, a desktop computer, a laptop, a cell phone, a personal digital assistant (PDA), a server, an embedded computing system, a mobile device and so forth.

Data processing system 18 can be a variety of computing devices capable of receiving data and running one or more services. In one embodiment, data processing system 18 can include a server, a distributed computing system, a desktop computer, a laptop, a cell phone, a rack-mounted server, and the like. Data processing system 18 can be a single server or a group of servers that are at a same position or at different positions (i.e., locations). Data processing system 18 and client device 12 can run programs having a client-server relationship to each other. Although distinct modules are shown in the figures, in some embodiments, client and server programs can run on the same device.

Data processing system 18 can receive data from wireless devices 14, and/or client device 12 through input/output (I/O) interface 24, and data repository 20. Data repository 20 can store a variety of data values for classifier data processing program 30. The classifier data processing program (which may also be referred to as a program, software, a software application, a script, or code) can be written in any form of programming language, including compiled or interpreted languages, or declarative or procedural languages, and it can be deployed in any form, including as a stand-alone program or as a module, component, subroutine, or other unit suitable for use in a computing environment. The classifier data processing program may, but need not, correspond to a file in a file system. The program can be stored in a portion of a file that holds other programs or information (e.g., one or more scripts stored in a markup language document), in a single file dedicated to the program in question, or in multiple coordinated files (e.g., files that store one or more modules, sub programs, or portions of code). The classifier data processing program can be deployed to be executed on one computer or on multiple computers that are located at one site or distributed across multiple sites and interconnected by a communication network.

In one embodiment, data repository 20 stores data 21 indicative of the gene-specific levels of mRNA, for example, the gene-specific levels of mRNA transcribed from each gene in the set of genes for a group of individuals who experienced the immune-related adverse event, a group of individuals who did not experience the immune-related adverse event, and/or a test subject. In another embodiment, data repository 20 stores parameters of a classifier, for example, coefficients and constants of a logistic regression equation. I/O interface 24 can be a type of interface capable of receiving data over a network, including, e.g., an Ethernet interface, a wireless networking interface, a fiber-optic networking interface, a modem, and so forth. Data processing system 18 also includes a processing device 28. As used herein, a “processing device” encompasses all kinds of apparatus, devices, and machines for processing information, including by way of example a programmable processor, a computer, or multiple processors or computers. The apparatus can include special purpose logic circuitry, e.g., an FPGA (field programmable gate array) or an ASIC (application specific integrated circuit) or RISC (reduced instruction set circuit). The apparatus can also include, in addition to hardware, code that creates an execution environment for the computer program in question, e.g., code that constitutes processor firmware, a protocol stack, an information base management system, an operating system, or a combination of one or more of them.

Data processing system 18 also includes memory 22 and a bus system 26, including, for example, a data bus and a motherboard, can be used to establish and to control data communication between the components of data processing system 18. Processing device 28 can include one or more microprocessors. Generally, processing device 28 can include an appropriate processor and/or logic that is capable of receiving and storing data, and of communicating over a network (not shown). Memory 22 can include a hard drive and a random access memory storage device, including, e.g., a dynamic random access memory, or other types of non-transitory machine-readable storage devices. Memory 22 stores classifier data processing program 30 that is executable by processing device 28. These computer programs may include a data engine (not shown) for implementing the operations and/or the techniques described herein. The data engine can be implemented in software running on a computer device, hardware or a combination of software and hardware.

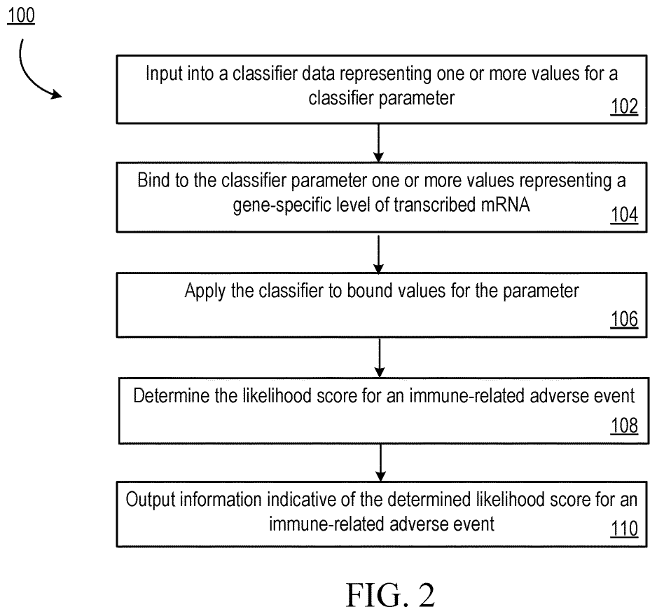

Referring to FIG. 2, data processing system 18 performs process 100 to output a likelihood score indicative of the probability of an immune-related adverse event associated with immunotherapy treatment. In operation, data processing system 18 inputs (102), into a classifier, data representing one or more values for a classifier parameter. The data can come from wireless devices 14, client device 12, and/or data repository 20. Data processing system 18 binds (104) one or more values representing a gene-specific level of transcribed mRNA to the classifier parameter. Data processing system 18 applies (106) the classifier to bound values for the parameter, and determines (108) a likelihood score indicating a probability of an immune-related adverse event associated with immunotherapy. Data processing system 18 outputs (110), by the one or more data processing devices 28, information (e.g., likelihood score) indicative of probability of an immune-related adverse event associated with immunotherapy. The output may be transmitted to a display device, e.g., a CRT (cathode ray tube) or LCD (liquid crystal display) monitor, or transmitted to client device 12, or wireless device 14 through network 16.

### Immunotherapies and Immune-Related Adverse Events

A number of new cancer immunotherapies have been approved by the Food & Drug Administration (FDA) for treating malignant melanoma, non-small cell lung cancer and kidney cancer. Based on the success of these results, clinical trials of new cancer immunotherapies are underway for many other types of cancer. The immune system has proteins called “checkpoint inhibitors” that control the immune system, preventing it from attacking normal tissue and thereby preventing autoimmune diseases. However, these checkpoint inhibitors can also allow cancer cells to escape immune system surveillance, leading to tumor proliferation. Creelan, Benjamin C. “Update on immune checkpoint inhibitors in lung cancer.” Cancer Control 21.1 (2014): 80-89. In certain patients, these recently approved cancer immunotherapies, including Yervoy® (ipilimumab) from Bristol Meyers Squibb and Keytruda® (pembrolizumab) from Merck, stimulate the immune system to “take the brakes off,” which helps the immune system recognize and attack cancer cells more effectively.

Despite important clinical benefits, immunotherapies are often associated with a unique spectrum of side effects termed immune-related adverse events. For example, in one study, immune-related adverse events were noted on in 31% of melanoma patients treated with ipilimumab (See Tirumani, Sree Harsha, et al. “Radiographic profiling of immune-related adverse events in advanced melanoma patients treated with ipilimumab.” Cancer immunology research 3.10 (2015): 1185-1192).

These immune-related adverse events can be local or systemic adverse reactions. They typically involve the gut, skin, endocrine glands, liver, or lung, and can potentially affect any other organs or tissue. The most frequent adverse events observed in at least one trial were rash, diarrhea, asthenia, nausea and headache (Ribas et al. “Phase III randomized clinical trial comparing tremelimumab with standard-of-care chemotherapy in patients with advanced melanoma.” Journal of Clinical Oncology 31.5 (2013): 616-622). Immune-related adverse events that involve skin can include, but are not limited to, pruritus, rash, rash maculopapular, rash erythematous, dermatitis, dermatitis acneiform, and vitiligo. Immune-related adverse events that involve the gastrointestinal system can include, but are not limited to, diarrhea and colitis. Immune-related adverse events that involve the liver can include, but are not limited to, increased serum alanine aminotransferase (ALT), increased serum aspartate aminotransferase (AST), and hepatitis. Immune-related adverse events that involve the endocrine glands can include, but are not limited to, hypothyroidism, hyperthyroidism, hypopituitarism, hypophysitis, adrenal insufficiency, increased thyrotropin, decreased corticotropin, increased amylase, and pancreatitis. Immune-related adverse events that involve the respiratory system can include, but are not limited to, dyspnea and pneumonitis. Immune-related adverse events that involve the kidney can include, but are not limited to, renal failure and increased serum creatinine. Other immune-related adverse events include, but are not limited to, fatigue, fever, chills, nausea, etc. Many of these immune related adverse events are further described, e.g., in Bertrand et al, “Immune related adverse events associated with anti-CTLA-4 antibodies: systematic review and meta-analysis,” BMC medicine 13.1 (2015): 1, which is incorporated by reference in its entirety.

A severe adverse reaction to an immunotherapy treatment (e.g., Grade 3 or Grade 4 diarrhea) will often require that the treatment be halted at least temporarily until the adverse reaction resolves, thereby potentially decreasing the effectiveness of the treatment in eliminating the patient's cancer. And even if the treatment is not halted, the adverse event will negatively affect the patent's quality of life, and if severe enough, require hospitalization and possibly even cause death. Knowing in advance that a given patient is likely to experience an immune-related adverse event upon receiving immunotherapy permits the caregiver to alter the patient's treatment plan to minimize the potency of the predicted adverse event, e.g., by monitoring for early indicators of the adverse event and then acting aggressively to minimize its severity by administering suitable therapies even before symptoms begin. Thus, a blood test to predict whether a cancer patient is likely to have an adverse reaction to ongoing cancer immunotherapy is of great clinical importance. In some embodiments, oncologists can use the methods described in the present disclosure to reduce the incidence or severity of adverse events in patients who receive immunotherapy, keeping them out of the hospital and improving their quality of life. Avoiding the hospitalization necessary to treat an adverse event such as severe diarrhea will also reduce the overall medical cost of immunotherapy.

In some embodiments, the immunotherapy targets any one of CD52, CTLA4, CD20, or programmed cell death 1 (PD-1) receptor. The immunotherapy treatment could be an immunomodulator, T-cell adoptive transfer, genetically engineered T cells, or an antibody immunotherapy (e.g., alemtuzumab, ipilimumab, ofatumumab, nivolumab, pembrolizumab, or rituximab). Among these antibody immunotherapies, alemtuzumab targets CD52, ipilimumab and tremelimumab target CTLA4, ofatumumab and rituximab target CD20, and nivolumab and pembrolizumab target programmed cell death 1 (PD-1) receptor. In some embodiments, the antibody immunotherapy is an anti-CTLA4 antibody, for example, ipilimumab (Yervoy®) or tremelimumab.

This disclosure provides methods of identifying immunotherapy patients who are at relatively high risk (compared to an average patient receiving the immunotherapy) of developing an immune-related adverse event. In some embodiments, if it is determined that a subject is likely to have an immune-related adverse event (e.g., Grade 2, Grade 3, or Grade 4 diarrhea, or colitis), the subject is thereafter closely monitored for the immune-related adverse event and/or receives a preventive treatment for the immune-related adverse event. For example, in these cases, the degree of monitoring for early symptoms related to development of the immune-related adverse event can be increased compared to the degree of monitoring (if any) for the early symptoms provided to the subject between the start of the immunotherapy and the time the patient was determined to be at a relatively high risk of developing an immune-related adverse event, or compared to the degree of monitoring for the early symptoms typically provided to patients undergoing treatment with the immunotherapy who have not been determined to be at relatively high risk of developing the immune-related adverse event. The monitoring typically provided to patients not determined to be at relatively high risk of developing diarrhea often is nothing more than giving the patient, at or before the start of immunotherapy and during subsequent visits during the treatment period, an explanation (orally or in writing or both) that some immunotherapy patients develop diarrhea at some point during therapy, that severe diarrhea can be dangerous, that the patient should self-monitor for changes in bowel habits and report any changes to the patient's medical provider, and/or that the patient should stay well-hydrated. The heightened degree of monitoring contemplated for patients identified as being at relatively high risk could include a warning that the patient is at relatively high risk coupled with a direction that the patient contact the medical provider every week (or every 5 days, or every 4 days, or every 3 days, or every 2 days, or every day) with an update as to symptoms such as changes in bowel habits or abdominal pain. The heightened degree of monitoring could include a program of having the medical provider or his/her proxy reach out to the patient on a frequent basis (e.g., every 7, 6, 5, 4, 3, or 2 days, or every day) to enquire about symptoms. The heightened degree of monitoring could include involving a gastroenterologist in the patient's care, or conducting endoscopy to search for early signs of intestinal inflammation.

In appropriate cases, the subject can receive a treatment suitable for reducing the likelihood the subject will actually experience an immune-related adverse event (e.g., Grade 2, Grade 3, or Grade 4 diarrhea, or colitis), or an exacerbation of Grade 1 diarrhea to a higher and thus more serious grade. In some cases, the dose of the immunotherapy can be reduced. In other cases, different routes of administration or different formulations can be used. In some cases, temporary immunosuppression with corticosteroids, tumor necrosis factor-alpha antagonists, mycophenolate mofetil, drugs of the kind typically given to suppress transplant rejection, or other immunosuppressive agents can be administered to the subject as a preventative measure, prior to development of any symptoms of the immune-related adverse event, or in the earliest stages of the adverse event before it has become serious, or after the appearance of symptoms of severe diarrhea but before test results have been obtained to rule out an infection as the cause of the diarrhea. (Normally the medical provider would await those test results before starting immunosuppressive therapy. Knowing in advance that the patient is in the high-risk group for developing immune-related Grade 2/3/4 diarrhea based on biomarkers would give the medical provider confidence to proceed with immunosuppressive therapy to control the diarrhea, without awaiting test results to rule out an infectious cause for the diarrhea.) In some embodiments, the doctor can select a different appropriate treatment regimen for the subject if the subject is determined to be likely to have an immune-related adverse event upon treatment with a particular immunotherapy, e.g., a severe immune-related adverse event.

The management of immune-related adverse events is described, e.g., in Tarhini, Ahmad. “Immune-mediated adverse events associated with ipilimumab ctla-4 blockade therapy: the underlying mechanisms and clinical management.” Scientifica 2013 (2013), which is incorporated by reference in its entirety.

### Immunotherapy-Induced Diarrhea/Colitis

Gastrointestinal adverse events, including diarrhea and/or colitis, are one of the most frequent categories of adverse reactions associated with immunotherapy. In many immunotherapy patients, diarrhea presents as moderate (Grade 2) diarrhea approximately 6 weeks after the initial administration of anti-CTLA4 or anti-PD-1 treatment and peaks as severe (Grade 3) and even life-threatening (Grade 4) later during treatment. As reported in one study of 945 patients with unresectable stage III or IV melanoma who received immunotherapy, Grade 3 or 4 diarrhea (Grade 2 diarrhea statistics not reported) occurred in 16.3% who received only nivolumab, 27.3% who received only ipilimumab, and 55.0% who received nivolumab-plus-ipilimumab. The incidence of diarrhea is reportedly much higher in patients receiving cytotoxic T-lymphocyte-associated antigen 4 (CTLA-4)-blocking antibodies compared with patients receiving immunotherapies that target the programmed cell death-1 (PD-1) receptor (Larkin et al. “Combined nivolumab and ipilimumab or monotherapy in untreated melanoma.” N Engl J Med 2015.373 (2015): 23-34).

The medical intervention for immunotherapy-induced severe diarrhea/colitis usually involves systemic corticosteroid treatment, hospitalization and anti-TNF-α-therapy. The caregiver normally first needs to rule out other causes of diarrhea, such as infections with Clostridium difficile or other bacterial/viral pathogens, as those require a different treatment approach.

The severity of immunotherapy induced diarrhea is often measured by Grades 1-4. Grade 1 refers to mild diarrhea, Grade 2 refers to moderate diarrhea, Grade 3 refers to severe diarrhea, and Grade 4 refers to potentially life-threatening diarrhea. Grade 0 means that the subject does not have observable diarrhea. These toxicity levels of diarrhea are described, e.g., in Food and Drug Administration, “Toxicity grading scale for healthy adult and adolescent volunteers enrolled in preventive vaccine clinical trials,” US Department of Health and Human Services (2007), which is incorporated by reference in its entirety.

TABLE 1
National Cancer Institute Common Toxicity Criteria for Diarrhea

| Diarrhea | Common Toxicity Criteria |
|---|---|
| Grade 1 (Mild) | 2-3 loose stools or <400 gms/24 hours |
| Grade 2 (Moderate) | 4-5 stools or 400-800 gms/24 hours |
| Grade 3 (Severe) | 6 or more watery stools or >800 gms/24 |
| | hours or requires outpatient IV hydration |
| Grade 4 (Potentially | ER visit or hospitalization |
| life threatening) | |

Mild (grade 1) diarrhea symptoms include 2-3 loose stools or <400 gms/24 hours. Grade 1 diarrhea has no interference with activity caused by headaches, fever or fatigue. It can be managed symptomatically. In some cases, anti-motility agents (loperamide or oral diphenoxylate atropine sulfate) are prescribed to the patients with mild symptoms. Budesonide can also be helpful for treating mild noninfectious diarrhea that persists but does not escalate after two to three days of dietary modification and treatment with anti-motility agents.

Moderate (grade 2) diarrhea symptoms include 4-5 loose stools or 400-800 gms/24 hours, and some interference with activity caused by headaches, fever (101° F.-102° F.), or fatigue. Colonoscopy may be helpful if grade 2 symptoms (increase of four to six stools per day over baseline) or greater occur or in situations where the diagnosis is unclear.

Severe (grade 3) diarrhea symptoms include 6 or more watery stools or >800 gms/24 hours, and headaches, fever (102.1° F.-104° F.), fatigue, and/or nausea/vomiting that significantly interferes with daily activity. Grade 3 diarrhea requires immediate medical attention. In some cases, Grade 3 diarrhea requires outpatient IV hydration and/or treatment with systemic steroids, and often includes hospitalization. In some cases, if Grade 3 diarrhea persists after 10 days of medical intervention, the patient is taken off the immunotherapy treatment.

Grade 4 diarrhea symptoms include an increase of seven or more stools per day over baseline or other complications, e.g., fever over 104° F. It is life threatening, requiring immediate emergency room treatment or hospitalization. Treatment with immunotherapy should be permanently discontinued. High doses of corticosteroids should be given to the patients.

If Grade 3-4 patients do not improve after approximately three days on intravenous corticosteroids, infliximab at a dose of 5 mg/kg once every two weeks is typically recommended. In cases refractory to infliximab, mycophenolate may be administered to the patient.

The immunotherapy can also induce colitis. In many cases, colitis is associated with Grades 3 or 4 diarrhea. Symptoms of colitis include, but are not limited to, mild to severe abdominal pain and tenderness, recurring bloody diarrhea with/without pus in the stools, fecal incontinence, flatulence, fatigue, loss of appetite and weight loss. More severe symptoms of colitis include, but are not limited to, shortness of breath, a fast or irregular heartbeat and fever. In some embodiments, patients with colitis are hospitalized and may receive a medication such as an anti-inflammatory agent or an immunosuppressant (e.g., a steroid). It is also important to keep the patient hydrated due to fluid loss.

In various aspects, the disclosure provides methods of identifying immunotherapy patients who are at higher risk of immunotherapy-induced diarrhea and/or immunotherapy-induced colitis than an average immunotherapy patient. In some embodiments, if an immunotherapy patient is predicted to be at risk of immunotherapy-induced diarrhea (e.g., Grade 2, Grade 3, or Grade 4 diarrhea) and/or immunotherapy-induced colitis, the patient will be placed in a heightened monitoring program more intense than is typically provided to patients who have not shown symptoms of diarrhea and have not been determined to be at higher than average risk of diarrhea and/or colitis. In such a heightened monitoring program, a health care provider will frequently contact the immunotherapy patient (e.g., by telephone or by email) or will ask the patient to come to the clinic on a frequent basis, to determine whether the patient has experienced any symptoms of an immune-related adverse event. In some of these cases, if the patient starts to experience some early symptoms of diarrhea (e.g., Grade 1 or Grade 2 diarrhea), the patient will be promptly treated with corticosteroid, such as budesonide, without the need to wait for results of additional tests to determine the cause of diarrhea (e.g., to determine whether the diarrhea is due to Clostridium difficile or other bacterial/viral pathogen infection), since the health care provider can be confident that the cause is the immunotherapy and not an infection. Thus, the health care provider can control diarrhea quickly before it worsens. The early treatment of the diarrhea will allow the immunotherapy patient to tolerate the immunotherapy longer and respond to the immunotherapy better and with less discomfort.

In some embodiments, if the immunotherapy patients are predicted to be at risk of immunotherapy-induced diarrhea and/or immunotherapy-induced colitis, the patients will also be asked to take necessary measures to prevent diarrhea/colitis caused by infection. These measures will allow the health care provider to be confident that any diarrhea that does develop is due to the immunotherapy and so can be treated immediately with appropriate immunosuppressive treatments, rather than waiting for the results of tests for infectious agents.

### Subject

A subject can include an individual who has been diagnosed as having cancer. In some embodiments, the subject is being treated with an immunotherapy.

Diagnosis of cancer can be made by lab tests and imaging techniques, for example, X-rays, CT scans, MRIs, PET and PET/CTs, ultrasound, and LDH testing, and biopsy, including shave, punch, incisional, and excisional biopsy.

In some embodiments, a subject can be someone who is suffering from any of various stages of cancer. Most types of cancer have 4 stages, numbered from 1 to 4. Stage 1 usually means that a cancer is relatively small and contained within the organ in which it started. Stage 2 usually means the cancer has not started to spread into surrounding tissue, but the tumor is larger than in stage 1. Stage 3 usually means the cancer is still larger. It may have started to spread into surrounding tissues, and there are cancer cells in the lymph nodes in the area. Stage 4 means the cancer has spread from where it started to another body organ. This is also called secondary or metastatic cancer.

In some embodiments, the subject has been previously treated with a surgical procedure for removing cancerous tissue.

In some embodiments, the subject has previously been treated with any one or more therapeutic treatments for cancer, alone or in combination with a surgical procedure for removing cancerous tissue. Therapeutic treatments for cancer are known in the art and include, but are not limited to, chemotherapy, immunotherapy, monoclonal antibody therapy, gene therapy, adoptive T-cell therapy, and vaccine therapy. In a further embodiment, the individual from whom a sample is obtained is a test subject for whom it is unknown whether the subject will respond to an immunotherapy, or whether the immunotherapy will induce an immune-related adverse event in the subject.

In some embodiments, an immunotherapy (e.g., tremelimumab) is administered by IV infusion once every 90 days for up to four cycles. The mechanism of action involves stimulation of an immune response, and there is a lag period before an adverse reaction to the immunotherapy can be observed. In some embodiments, diarrhea and/or colitis may develop a few weeks or a few months after the first immunotherapy dose is administered to the subject. The biological sample (e.g., a blood sample) used in the presently described methods is typically collected after the start of the immunotherapy treatment, e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or30 days after the first dose of immunotherapy, or at least 1, 2, 3, 4, 5, 6, 7, 8, 9, 10, 11, or 12 weeks after the first dose of immunotherapy. In some cases, the blood sample will be collected within 6 weeks after the start of immunotherapy, typically before 5 weeks have elapsed after the start, and usually at or around a month (30 or 31 days) after the start of immunotherapy. The timing of taking that sample is independent of appearance of diarrhea symptoms, and generally will occur before any symptoms appear. In addition to that sample, or instead of it, a blood sample may be collected shortly after the patient has first experienced Grade 1 (mild) diarrhea symptoms, e.g., at the first sign of diarrhea and before it progresses to Grade 2 or above. The first symptoms of diarrhea associated with immunotherapy typically do not appear until at least 6 weeks after the start of immunotherapy, so a blood sample taken at the earliest appearance of diarrhea symptoms will usually be taken at 6 weeks or later, but the timing of this sample is linked to when mild symptoms first appear, and not to a particular time period after start of immunotherapy. Patients will normally be asked to provide the blood sample very shortly (e.g., within a day or two) after the first diarrhea symptom is detected. However, a given patient may not immediately report the start of diarrhea symptoms to the caregiver, or may delay in providing the blood sample, so there may be a gap of several days or even a week or more from the start of the symptoms to the time the blood sample is collected. The sample is preferably taken before the diarrhea progresses to Grade 2 or higher.

In various aspects, the disclosure also provides methods of identifying a group of immunotherapy patients for clinical trials, where the clinical trial is intended to assess the efficacy of a treatment intended to prevent or reduce the incidence or severity of immune-related adverse events in patients being treated with an immunotherapy. By selecting for patients who are most at risk of experiencing the immune-related adverse event during immunotherapy, and including only those selected patients in the clinical trial, the trial can be powered to show statistically significant efficacy of the treatment with fewer total patients than if the selection for high-risk patients was not done. In these methods, patients undergoing immunotherapy for cancer would be tested to ascertain whether they are at increased risk for an immune-related adverse event (such as Grade 3/4 diarrhea or Grade 2/3/4 diarrhea, and/or colitis), prior to experiencing such an event. Patients who are diagnosed as being at increased risk would be included in a clinical trial intended to test the efficacy of a co-treatment (given in conjunction with the immunotherapy) intended to reduce the likelihood the patients will actually experience the immune-related adverse event. Patients who are diagnosed as not being at increased risk would be excluded from the clinical trial. They would continue to receive the immunotherapy without the co-treatment.

### Sample Preparation

Samples for use in the techniques described herein include any of various types of biological molecules, cells and/or tissues that can be isolated and/or derived from a subject. The sample can be isolated and/or derived from any fluid, cell or tissue. The sample can also be one isolated and/or derived from any fluid and/or tissue that predominantly comprises blood cells.

The sample that is isolated and/or derived from a subject can be assayed for gene expression products. In one embodiment, the sample is a fluid sample, a lymph sample, a lymph tissue sample or a blood sample. In one embodiment, the sample is isolated and/or derived from peripheral blood. In other embodiments, the sample may be isolated and/or derived from alternative sources, including from any one of various types of lymphoid tissue.

In one embodiment, a sample of blood is obtained from an individual according to methods well known in the art. In some embodiments, a drop of blood is collected from a simple pin prick made in the skin of an individual. Blood may be drawn from an individual from any part of the body (e.g., a finger, hand, wrist, arm, leg, foot, ankle, abdomen, or neck) using techniques known to one of skill in the art, such as phlebotomy. Examples of samples isolated and/or derived from blood include samples of whole blood, serum-reduced whole blood, serum-depleted blood, and serum-depleted and erythrocyte-depleted blood.

In some embodiments, whole blood collected from an individual is fractionated (i.e., separated into components) before measuring the absolute and/or relative abundance (level) of a biomarker in the sample. In one embodiment, blood is serum-depleted (or serum-reduced). In other embodiments, the blood is plasma-depleted (or plasma-reduced). In yet other embodiments, blood is erythrocyte-depleted or erythrocyte-reduced. In some embodiments, erythrocyte reduction is performed by preferentially lysing the red blood cells. In other embodiments, erythrocyte depletion or reduction is performed by lysing the red blood cells and further fractionating the remaining cells. In yet other embodiments, erythrocyte depletion or reduction is performed, but the remaining cells are not further fractionated. In other embodiments, blood cells are separated from whole blood collected from an individual using other techniques known in the art. For example, blood collected from an individual can be subjected to Ficoll-Hypaque™ (Pharmacia) gradient centrifugation to separate various types of cells in a blood sample. In particular, Ficoll-Hypaque™ gradient centrifugation is useful to isolate peripheral blood leukocytes (PBLs).

### RNA Quantification

The level of a biomarker (e.g., RNA) can be determined by any means known in the art, and can be taken to represent the level of expression of the corresponding gene. The quantity of RNA can be determined by various means, for example, by microarray (e.g., RNA microarray, cDNA microarray), quantitative polymerase chain reaction (qPCR), or sequencing technology (e.g., RNA-Seq).

In some embodiments, a level of a biomarker (when referring to RNA) is stated as a number of PCR cycles required to reach a threshold amount of RNA or DNA, e.g., 10, 15, 20, 25, 30, 35, 40, 45, 50, 55, or 60 cycles. The level of a biomarker, when referring to RNA, can also refer to a measurable quantity of a given nucleic acid as determined relative to the amount of total RNA, or cDNA used in QRT-PCR, in which the amount of total RNA used is, for example, 100 ng, 50 ng, 25 ng, 10 ng, 5 ng, 1.25 ng, 0.05 ng, 0.3 ng, 0.1 ng, 0.09 ng, 0.08 ng, 0.07 ng, 0.06 ng, or 0.05 ng. The level of a nucleic acid can be determined by any methods known in the art. For microarray analysis, the level of a nucleic acid is measured by hybridization analysis using nucleic acids corresponding to RNA isolated from the samples, according to methods well known in the art. The label used in the samples can be a luminescent label, an enzymatic label, a radioactive label, a chemical label or a physical label. In some embodiments, target and/or probe nucleic acids are labeled with a fluorescent molecule. The level of a biomarker, when referring to RNA, can also refer to a measurable quantity of a given nucleic acid as determined relative to the amount of total RNA or cDNA used in a microarray hybridization assay. In some embodiments, the amount of total RNA is μg, 5 μg, 2.5 μg, 2 μg, 1 μg, 0.5 μg, 0.1 μg, 0.05 μg, 0.01 μg, 0.005 μg, 0.001 μg, or the like. In some embodiments, the level of a biomarker, when referring to RNA, can refer to the number of mapped reads identified by RNA-Seq. The reads can be further normalized, e.g., by the total number of mapped reads, so that biomarker levels are expressed as Fragments Per Kilobase of transcript per Million mapped reads (FPKM).

In some embodiments, RNA is obtained from the nucleic acid mix using a filter-based RNA isolation system such as that from Ambion (RNAqueous™, Phenol-free Total RNA Isolation Kit, Catalog #1912, version 9908; Austin, Tex.) or the PAXgene™ Blood RNA System (from Pre-Analytix). The detailed method is described in pp. 55-104, in RNA Methodologies, A laboratory guide for isolation and characterization, 2nd edition, 1998, Robert E. Farrell, Jr., Ed., Academic Press. In some embodiments, RNA is prepared using a well-known system for isolating RNA (including isolating total RNA or mRNA, and the like) such as oligo dT based purification methods, Qiagen® RNA isolation methods, LeukoLOCKT™ Total RNA Isolation System, MagMAXM-96 Blood Technology from Ambion, Promega® polyA mRNA isolation system, and the like.

In some embodiments, the level of transcribed mRNA can be quantified by quantitative real-time PCR (QRT-PCR), for example, with an Applied Biosystem Prism® instrument, Cepheid SmartCycler® instrument, Cepheid GeneXpert® instrument or the Roche LightCycler® 480 Real-Time PCR System.

### Genes Measured in the Studies

The mRNA expressed from each of a total of 168 genes was measured in blood samples from all subjects in the two studies. The short name, full name, and aliases for each of these genes are listed in Table 2.

TABLE 2
List of Genes with Full Name and Aliases of Each

| Gene | Full Name | Aliases |
|---|---|---|
| ADAM17 | ADAM Metallopeptidase Domain 17 | ADAM18, CD156B, NISBD1, NISBD, TACE, CSVP |
| ALOX5 | Arachidonate 5-Lipoxygenase | 5-LOX, 5LPG, LOX-5, 5-LO, LOG5, 5-lipoxygenase |
| ANLN | Anillin, Actin Binding Protein | FSGS8, Scraps, scra |
| APAF1 | Apoptotic Peptidase Activating | CED4, APAF-1, KIAA0413 |
| | Factor 1 | |
| AXIN2 | Axin 2 | conductin, axil, axin-2, ODCRCS |
| BAD | BCL2-Associated Agonist Of Cell | BBC2, bcl2-L-8, BCL2L8, BBC6 |
| | Death | |
| BAX | BCL2-Associated X Protein | Baxdelta2G9, Baxdelta2G9omega, Baxdelta2omega, |
| | | bcl2-L-4, BCL2L4 |
| BLVRB | Biliverdin Reductase B | FR, GHBP, HEL-S-10, SDR43U1, BVRB, FLR, BVR-B |
| BPGM | 2,3-Bisphosphoglycerate Mutase | DPGM |
| BRCA1 | Breast Cancer 1, Early Onset | BRCC1, BRCAI, FANCS, IRIS, PPP1R53, BROVCA1, |
| | | PNCA4, PSCP, RNF53 |
| C1QA | Complement Component 1, Q | |
| | Subcomponent, A Chain | |
| CASP1 | Caspase 1, Apoptosis-Related | p45, caspase-1, beta, convertase, IL1B-convertase, |
| | Cysteine Peptidase | IL1BC, ICE, CASP-1, IL-1BC, IL1BCE |
| CASP3 | Caspase 3, Apoptosis-Related | CPP32B, apopain, caspase-3, procaspase3, CPP32, |
| | Cysteine Peptidase | CASP-3, CPP-32, SCA-1 |
| CCL3 | Chemokine (C-C Motif) Ligand 3 | LD78ALPHA, SCYA3, MIP1A, G0S19-1, MIP-1-alpha, |
| | | SIS-beta |
| CCL5 | Chemokine (C-C Motif) Ligand 5 | SIS-delta, RANTES, SISd, eoCP, D17S136E, SCYA5, |
| | | TCP228 |
| CCND1 | Cyclin D1 | U21B31, D11S287E, BCL1, PRAD1, BCL-1 |
| CCR3 | Chemokine (C-C Motif) Receptor 3 | CD193, CMKBR3, CKR3, CC-CKR-3, CCR-3 |
| CCR5 | Chemokine (C-C Motif) Receptor 5 | ChemR13, CD195, CKR-5, CKR5, CCCKR5, IDDM22, |
| | (Gene/Pseudogene) | CMKBR5, CC-CKR-5, CCR-5 |
| CCR7 | Chemokine (C-C Motif) Receptor 7 | CD197, CMKBR7, EBI1, BLR2, CC-CKR-7, CCR-7, |
| | | CDw197, EVI1 |
| CCR9 | Chemokine (C-C Motif) Receptor 9 | CDw199, GPR28, CC-CKR-9, GPR-9-6, CCR-9 |
| CD19 | CD19 Molecule | B4, CVID3 |
| CD28 | CD28 Molecule | Tp44 |
| CD4 | CD4 Molecule | CD4mut |
| CD40 | CD40 Molecule, TNF Receptor | P50, CDW40, TNFRSF5, Bp50 |
| | Superfamily Member 5 | |
| CD40LG | CD40 Ligand | TRAP, CD154, T-BAM, gp39, hCD40L, IMD3, IGM, |
| | | HIGM1, TNFSF5, CD40-L, CD40L |
| CD80 | CD80 Molecule | B7-1, B7.1, CD28LG1, CD28LG, LAB7, B7, BB1 |
| CD86 | CD86 Molecule | B7-2, B7.2, LAB72, CD28LG2, B70, BU63, FUN-1 |
| CD8A | CD8a Molecule | Leu2, p32, CD8, MAL |
| CDC25A | Cell Division Cycle 25A | CDC25A2 |
| CDH1 | Cadherin 1, Type 1, E-Cadherin | E-Cadherin, Arc-1, CD324, cadherin-1, uvomorulin, |
| | (Epithelial) | ECAD, LCAM, UVO, CDHE, uvomorulin) |
| CDK2 | Cyclin-Dependent Kinase 2 | CDKN2, p33(CDK2) |
| CDKN1A | Cyclin-Dependent Kinase Inhibitor | CIP1, P21, p21CIP1, CDKN1, WAF1, CAP20, MDA-6, |
| | 1A (P21, Cip1) | SDI1, MDA6, PIC1 |
| CDKN1B | Cyclin-Dependent Kinase Inhibitor | MEN1B, P27KIP1, CDKN4, MEN4, KIP1 |
| | 1B (P27, Kip1) | |
| CDKN2A | Cyclin-Dependent Kinase Inhibitor | CDK4I, p19, INK4, INK4A, P14, P14ARF, P16-INK4A, |
| | 2A | P16INK4, P16INK4A, P19ARF, TP16, CDKN2, CMM2, |
| | | P16, MLM, MTS1, ARF, MTS-1, p16-INK4 |
| CDKN2D | Cyclin-Dependent Kinase Inhibitor | INK4D, p19, p19-INK4D |
| | 2D (P19, Inhibits CDK4) | |
| CHPT1 | Choline Phosphotransferase 1 | CPT, CPT1, hCPT1 |
| CIITA | Class II, Major Histocompatibility | CIITAIV, NLRA, C2TA, MHC2TA |
| | Complex, Transactivator | |
| CNKSR2 | Connector Enhancer Of Kinase | MAGUIN, CNK2, KSR2, KIAA0902 |
| | Suppressor Of Ras 2 | |
| CSF2 | Colony Stimulating Factor 2 | sargramostim, molgramostin, GMCSF, CSF, GM-CSF |
| | (Granulocyte-Macrophage) | |
| CTLA4 | Cytotoxic T-Lymphocyte-Associated | ALPS5, CD, GSE, GRD4, CELIAC3, IDDM12, CD152, |
| | Protein 4 | CTLA-4 |
| CTSD | Cathepsin D | ceroid-lipofuscinosis, HEL-S-130P, CLN10, CPSD |
| CXCL1 | Chemokine (C-X-C Motif) Ligand 1 | GROa, MGSA-a, FSP, GRO1, MGSA, GRO-alpha(1-73), |
| | (Melanoma Growth Stimulating | NAP-3, SCYB1, alpha), GRO |
| | Activity, Alpha) | |
| CXCL10 | Chemokine (C-X-C Motif) Ligand 10 | C7, IFI10, crg-2, gIP-10, gamma-IP10, mob-1, SCYB10, |
| | | INP10, IP-10 |
| CXCL8 | Chemokine (C-X-C Motif) Ligand 8 | NAP1, GCP1, LECT, LUCT, LYNAP, NAF, emoctakin, |
| | | interleukin-8, IL8, GCP-1, MDNCF, MONAP, NAP-1, IL-8 |
| CXCR3 | Chemokine (C-X-C Motif) Receptor 3 | CD182, CD183, CMKAR3, IP10-R, Mig-R, MigR, GPR9, |
| | | CKR-L2, CXC-R3, CXCR-3 |
| DENND2D | DENN/MADD Domain Containing 2D | RP5-1180E21.2 |
| DLC1 | DLC1 Rho GTPase Activating Protein | DLC-1, HP, p122-RhoGAP, ARHGAP7, STARD12, |
| | | KIAA1723 |
| DPP4 | Dipeptidyl-Peptidase 4 | DPPIV, ADCP2, CD26, ADABP, ADCP-2, TP103 |
| E2F1 | E2F Transcription Factor 1 | RBAP1, RBP3, RBBP3, E2F-1, PBR3, RBAP-1, RBBP-3 |
| EGR1 | Early Growth Response 1 | G0S30, KROX-24, TIS8, ZIF-268, AT225, EGR-1, NGFI-A, |
| | | ZNF225, KROX24 |
| ELANE | Elastase, Neutrophil Expressed | medullasin, GE, HNE, NE, PMN-E, elastase-2, SCN1, |
| | | ELA2, HLE |
| ERBB2 | Erb-B2 Receptor Tyrosine Kinase 2 | CD340, HER-2, HER-2/neu, TKR1, herstatin, NGL, HER2, |
| | | NEU, p185erbB2, MLN19 |
| F5 | Coagulation Factor V (Proaccelerin, | FVL, PCCF, RPRGL1, THPH2 |
| | Labile Factor) | |
| FAIM3 | Fas Apoptotic Inhibitory Molecule 3 | FCMR, TOSO |
| FAM210B | Family With Sequence Similarity | 5A3, dJ1167H4.1, C20orf108 |
| | 210, Member B | |
| FASLG | Fas Ligand (TNF Superfamily, | CD178, ALPS1B, APT1LG1, TNFSF6, FASL, APTL, CD95- |
| | Member 6) | L, CD95L |
| FCGR2B | Fc Fragment Of IgG, Low Affinity IIb, | CDW32, CD32, FCG2, IGFR2, CD32B, fc-gamma-RIIb, |
| | Receptor (CD32) | fcRII-b, FCGR2 |
| FOS | FBJ Murine Osteosarcoma Viral | p55, AP-1, C-FOS, G0S7 |
| | Oncogene Homolog | |
| FOXP3 | Forkhead Box P3 | DIETER, FOXP3delta7, JM2, scurfin, AIID, PIDX, XPID, |
| | | IPEX |
| FYN | FYN Proto-Oncogene, Src Family | SYN, p59-FYN, SLK |
| | Tyrosine Kinase | |
| GADD45A | Growth Arrest And DNA-Damage- | DDIT1, GADD45, DDIT-1 |
| | Inducible, Alpha | |
| GLRX5 | Glutaredoxin 5 | GRX5, PR01238, PRSA, FLB4739, PRO1238, C14orf87 |
| GYPA | Glycophorin A (MNS Blood Group) | MN, CD235a, GPErik, GPSAT, HGpMiV, HGpMiXI, |
| | | HGpSta(C), glycophorin-A, MNS, GPA, PAS-2 |
| GYPB | Glycophorin B (MNS Blood Group) | MNS, CD235b, glycophorin-B, SS, GPB, PAS-3 |
| GZMA | Granzyme A (Granzyme 1, Cytotoxic | fragmentin-1, CTLA3, HFSP, HF, Granzyme-1 |
| | T-Lymphocyte-Associated Serine | |
| | Esterase 3) | |
| GZMB | Granzyme B (Granzyme 2, Cytotoxic | HLP, CTLA1, CCPI, CGL-1, CSP-B, fragmentin-2, CSPB, |
| | T-Lymphocyte-Associated Serine | C11, CGL1, CTLA-1, CTSGL1, SECT, GRB, Granzyme-2 |
| | Esterase 1) | |
| HLA-DRA | Major Histocompatibility Complex, | MLRW, HLA-DRA1 |
| | Class II, DR Alpha | |
| HMGA1 | High Mobility Group AT-Hook 1 | HMG-R, HMGA1A, HMGIY, HMG-I(Y) |
| HMGB1 | High Mobility Group Box 1 | Amphoterin, HMG3, SBP-1, HMG1, HMG-1 |
| HMOX1 | Heme Oxygenase 1 | HSP32, bK286B10, HMOX1D, HO-1, HO, HO1 |
| HOXA10 | Homeobox A10 | PL, HOX1.8, HOX1, HOX1H |
| HSPA1A | Heat Shock 70 kDa Protein 1A | HEL-S-103, HSP70-1, HSP70-1A, HSP70I, HSP72, |
| | | HSPA1, HSP70-1/HSP70-2, HSP70.1/HSP70.2, HSX70 |
| ICAM1 | Intercellular Adhesion Molecule 1 | BB2, CD54, P3.58, ICAM-1 |
| ICOS | Inducible T-Cell Co-Stimulator | CD278, CVID1, AILIM |
| IFI16 | Interferon, Gamma-Inducible | PYHIN2, IFNGIP1, Ifi-16 |
| | Protein 16 | |
| IFNG | Interferon, Gamma | IFG, IFI, IFN-gamma |
| IGF2BP2 | Insulin-Like Growth Factor 2 MRNA | IMP2, IMP-2, VICKZ2 |
| | Binding Protein 2 | |
| IGHG2 | Immunoglobulin Heavy Constant | |
| | Gamma 2 (G2m Marker) | |
| IL10 | Interleukin 10 | IL10A, TGIF, interleukin-10, GVHDS, CSIF, IL-10 |
| IL12B | Interleukin 12B | p40, IL12, CLMF, CLMF2, IMD28, IMD29, NKSF, NKSF2, |
| | | IL-12B |
| IL15 | Interleukin 15 | interleukin-15, IL-15 |
| IL18 | Interleukin 18 | IL-1g, iboctadekin, interleukin-18, IGIF, IL-18, IL1F4 |
| IL18BP | Interleukin 18 Binding Protein | IL18BPa, tadekinig-alfa, IL-18BP |
| IL1B | Interleukin 1, Beta | IL-1, IL1-BETA, catabolin, pro-interleukin-1-beta, IL1F2 |
| IL1R1 | Interleukin 1 Receptor, Type I | p80, IL1RA, CD121A, D2S1473, IL1R, IL-1R-1, IL-1R- |
| | | alpha, IL-1RT-1, IL-1RT1, IL1RT1 |
| IL1R2 | Interleukin 1 Receptor, Type II | CD121b, IL1R2C, IL1RB, CDw121b, IL-1R-2, IL-1R-beta, |
| | | IL-1RT-2, IL-1RT2 |
| IL1RN | Interleukin 1 Receptor Antagonist | IL-1ra3, DIRA, MVCD4, ICIL-1RA, IL-1RN, IL-1ra, IL1F3, |
| | | IL1RA, IRAP, Anakinra |
| IL2 | Interleukin 2 | aldesleukin, interleukin-2, lymphokine, IL-2, TCGF |
| IL23A | Interleukin 23, Alpha Subunit P19 | p19, interleukin-six, IL-23, IL-23A, IL23P19, SGRF, IL- |
| | | 23-A, IL-23p19 |
| IL2RA | Interleukin 2 Receptor, Alpha | p55, CD25, TCGFR, IDDM10, IL2R, IL-2-RA, IL2-RA |
| IL32 | Interleukin 32 | IL-32alpha, IL-32beta, IL-32delta, IL-32gamma, TAIFa, |
| | | TAIFb, TAIFc, TAIFd, interleukin-32, NK4, TAIF, IL-32 |
| IL5 | Interleukin 5 | TRF, interleukin-5, eosinophil, EDF, IL-5 |
| IL6 | Interleukin 6 | interferon, interleukin-6, BSF2, HGF, HSF, IFNB2, BSF- |
| | | 2, CDF, IFN-beta-2, IL-6 |
| IL7R | Interleukin 7 Receptor | CDW127, ILRA, CD127, IL7RA, IL-7R-alpha, IL-7RA |
| INPP4B | Inositol Polyphosphate-4- | |
| | Phosphatase, Type II, 105 kDa | |
| IRAK3 | Interleukin-1 Receptor-Associated | ASRT5, IRAKM, IRAK-3, IRAK-M |
| | Kinase 3 | |
| IRF1 | Interferon Regulatory Factor 1 | MAR, IRF-1 |
| ITGA4 | Integrin, Alpha 4 (Antigen CD49D, | IA4, CD49D |
| | Alpha 4 Subunit Of VLA-4 Receptor) | |
| ITGAL | Integrin, Alpha L (Antigen CD11A | LFA-1, LFA1A, CD11A, LFA-1A |
| | (P180), Lymphocyte Function- | |
| | Associated Antigen 1; Alpha | |
| | Polypeptide) | |
| LARGE | Like-Glycosyltransferase | like-glycosyltransferase, MDC1D, MDDGA6, MDDGB6, |
| | | KIAA0609, LARGE1 |
| LCK | LCK Proto-Oncogene, Src Family | Lsk, IMD22, YT16, p56lck, pp58lck, p56-LCK |
| | Tyrosine Kinase | |
| LGALS3 | Lectin, Galactoside-Binding, Soluble, | L31, CBP35, GAL3, GALIG, galectin-3, LGALS2, GALBP, |
| | 3 | MAC2, Gal-3, L-31 |
| LTA | Lymphotoxin Alpha | LT, lymphotoxin-alpha, TNFB, LT-alpha, TNF-beta, |
| | | TNFSF1 |
| MAPK14 | Mitogen-Activated Protein Kinase | P38, RK, CSBP, EXIP, Mxi2, PRKM14, PRKM15, |
| | 14 | p38ALPHA, CSBP2, CSPB1, CSBP1, SAPK2A |
| MCAM | Melanoma Cell Adhesion Molecule | Gicerin, CD146, MUC18 |
| MIF | Macrophage Migration Inhibitory | GLIF, GIF, MMIF |
| | Factor (Glycosylation-Inhibiting | |
| | Factor) | |
| MMP12 | Matrix Metallopeptidase 12 | HME, ME, MME, MMP-12 |
| MMP9 | Matrix Metallopeptidase 9 | MANDP2, CLG4B, GELB, MMP-9 |
| MNDA | Myeloid Cell Nuclear Differentiation | PYHIN3 |
| | Antigen | |
| MSH2 | MutS Homolog 2 | HNPCC, LCFS2, FCC1, HNPCC1, COCA1, hMSH2 |
| MYC | V-Myc Avian Myelocytomatosis Viral | MRTL, MYCC, c-Myc, bHLHe39 |
| | Oncogene Homolog | |
| NAB2 | NGFI-A Binding Protein 2 (EGR1 | MADER |
| | Binding Protein 2) | |
| NBEA | Neurobeachin | LYST2, neurobeachin, BCL8B, KIAA1544 |
| NEDD4L | Neural Precursor Cell Expressed, | NEDD4-2, hNEDD4-2, RSP5, NEDD4.2, KIAA0439, |
| | Developmentally Down-Regulated | NEDL3 |
| | 4-Like, E3 Ubiquitin Protein Ligase | |
| NEDD9 | Neural Precursor Cell Expressed, | P105, Cas-like, CAS2, HEF1, CASL, CAS-L, CASS2, NEDD- |
| | Developmentally Down-Regulated 9 | 9 |
| NFATC1 | Nuclear Factor Of Activated T-Cells, | NF-ATC, NFAT2, NFATc, NF-ATc1, NFATc1 |
| | Cytoplasmic, Calcineurin-Dependent | |
| | 1 | |
| NFKB1 | Nuclear Factor Of Kappa Light | P50, P105, KBF1, NF-kB1, NF-kappa-B, NF-kappaB, NF- |
| | Polypeptide Gene Enhancer In B- | kappabeta, NFKB-p105, NFKB-p50, NFkappaB, EBP-1 |
| | Cells 1 | |
| NLRC4 | NLR Family, CARD Domain | AIFEC, CLANA, CLANB, CLANC, CLAND, CLR2.1, FCAS4, |
| | Containing 4 | CARD12, CLAN, IPAF, CLAN1 |
| NME4 | NME/NM23 Nucleoside | NDK, NDPK-D, NM23H4, NDPKD, nm23-H4, NM23D |
| | Diphosphate Kinase 4 | |
| NRAS | Neuroblastoma RAS Viral (V-Ras) | HRAS1, CMNS, N-ras, NCMS, NRAS1, ALPS4, NS6 |
| | Oncogene Homolog | |
| NUCKS1 | Nuclear Casein Kinase And Cyclin- | P1, JC7, NUCKS |
| | Dependent Kinase Substrate 1 | |
| NUDT4 | Nudix (Nucleoside Diphosphate | DIPP2alpha, DIPP2beta, HDCMB47P, DIPP-2, DIPP2, |
| | Linked Moiety X)-Type Motif 4 | KIAA0487 |
| PBX1 | Pre-B-Cell Leukemia Homeobox 1 | PRL |
| PDE3B | Phosphodiesterase 3B, CGMP- | HcGIP1, cGIPDE1, CGIP1 |
| | Inhibited | |
| PDGFA | Platelet-Derived Growth Factor | PDGF-A, PDGF-1, PDGF1 |
| | Alpha Polypeptide | |
| PLA2G7 | Phospholipase A2, Group VII | LDL-PLA2, LP-PLA2, PAFAD, PAFAH, LDL-PLA(2), gVIIA- |
| | (Platelet-Activating Factor | PLA2 |
| | Acetylhydrolase, Plasma) | |
| PLAUR | Plasminogen Activator, Urokinase | CD87, URKR, U-PAR, UPAR, MO3 |
| | Receptor | |
| PLEK2 | Pleckstrin 2 | pleckstrin-2 |
| PLXDC2 | Plexin Domain Containing 2 | 1200007L24Rik, TEM7R |
| PPP2R4 | Protein Phosphatase 2A Activator, | PTPA, PP2A, PR53 |
| | Regulatory Subunit 4 | |
| PTEN | Phosphatase And Tensin Homolog | 10q23del, DEC, PTEN1, BZS, MHAM, CWS1, GLM2, |
| | | MMAC1, TEP1 |
| PTGS2 | Prostaglandin-Endoperoxide | COX2, PGG/HS, GRIPGHS, PHS-2, hCox-2, COX-2, |
| | Synthase 2 (Prostaglandin G/H | PGHS-2, Cyclooxygenase-2 |
| | Synthase And Cyclooxygenase) | |
| PTPRC | Protein Tyrosine Phosphatase, | LCA, GP180, B220, CD45R, LY5, CD45, L-CA, T200 |
| | Receptor Type, C | |
| PTPRK | Protein Tyrosine Phosphatase, | R-PTP-kappa, PTPK |
| | Receptor Type, K | |
| RBM5 | RNA Binding Motif Protein 5 | G15, H37, LUCA15, RMB5 |
| RHOC | Ras Homolog Family Member C | H9, RHOH9, ARH9, ARHC |
| S100A4 | S100 Calcium Binding Protein A4 | MTS1, 18A2, 42A, FSP1, P9KA, PEL98, CAPL, |
| | | Calvasculin, Metastasin |
| S100A6 | S100 Calcium Binding Protein A6 | 2A9, 5B10, CABP, calcyclin, CACY, PRA |
| SCN3A | Sodium Channel, Voltage Gated, | Nav1.3, NAC3, KIAA1356 |
| | Type III Alpha Subunit | |
| SERPINA1 | Serpin Peptidase Inhibitor, Clade A | PI, alpha-1-antitrypsin, A1A, A1AT, PI1, PRO2275, |
| | (Alpha-1 Antiproteinase, | alpha-1-antiproteinase, alpha1AT, AAT |
| | Antitrypsin), Member 1 | |
| SERPINE1 | Serpin Peptidase Inhibitor, Clade E | PAI, PAI1, PLANH1, PAI-1, PLANH1 |
| | (Nexin, Plasminogen Activator | |
| | Inhibitor Type 1), Member 1 | |
| SIAH2 | Siah E3 Ubiquitin Protein Ligase 2 | siah-2, hSiah2 |
| SLC4A1 | Solute Carrier Family 4 (Anion | BND3, CD233, EMPB3, FR, RTA1A, SW, WD1, WR, WD, |
| | Exchanger), Member 1 (Diego Blood | DI, AE1, EPB3 |
| | Group) | |
| SOCS1 | Suppressor Of Cytokine Signaling 1 | CIS1, CISH1, SSI1, JAB, SOCS-1, SSI-1, TIP-3, TIP3 |
| SOCS3 | Suppressor Of Cytokine Signaling 3 | ATOD4, Cish3, CIS3, SSI3, SOCS-3, SSI-3, CIS-3 |
| SPARC | Secreted Protein, Acidic, Cysteine- | osteonectin, ON, BM-40 |
| | Rich (Osteonectin) | |
| ST14 | Suppression Of Tumorigenicity 14 | epithin, matriptase, HAI, TMPRSS14, prostamin, |
| | (Colon Carcinoma) | PRSS14, ARCI11, MTSP1, MT-SP1, SNC19, TADG15 |
| TGFB1 | Transforming Growth Factor, Beta 1 | LAP, CED, TGFbeta, TGFB, DPD1, TGF-beta-1 |
| THBS1 | Thrombospondin 1 | TSP1, thrombospondin-1p180, THBS, THBS-1, TSP-1, |
| | | thrombospondin-1, TSP |
| TIMP1 | TIMP Metallopeptidase Inhibitor 1 | EPO, HCI, CLGI, TIMP, EPA, TIMP-1 |
| TLK2 | Tousled-Like Kinase 2 | PKU-ALPHA, HsHPK |
| TLR2 | Toll-Like Receptor 2 | CD282, TIL4 |
| TLR4 | Toll-Like Receptor 4 | CD284, TLR-4, TOLL, ARMD10, hToll |
| TLR9 | Toll-Like Receptor 9 | CD289 |
| TMOD1 | | ETMOD, e-tropomodulin, tropomodulin-1, D9S57E, |
| | | TMOD, E-Tmod |
| TNF | Tumor Necrosis Factor | DIF, cachectin, TNFA, TNF-a, TNF-alpha, TNFSF2 |
| TNFRSF13B | Tumor Necrosis Factor Receptor | CD267, CVID, IGAD2, RYZN, TNFRSF14B, CVID2, TACI |
| | Superfamily, Member 13B | |
| TNFRSF1A | Tumor Necrosis Factor Receptor | P60, tbp1, p55, CD120a, TNF-R, TNF-R-I, TNF-R55, |
| | Superfamily, Member 1A | TNFR1-d2, TNFR55, TNFR60, p55-R, FPF, MS5, TNFR1, |
| | | TNFAR, TNF-R1, TNF-RI, TNFR-I |
| TNFRSF1B | Tumor Necrosis Factor Receptor | p75, CD120b, TBPII, TNF-R-II, TNF-R75, TNFR1B, |
| | Superfamily, Member 1B | TNFR80, p75TNFR, TNFR2, TNFBR, TNF-R2, TNF-RII, |
| | | Etanercept, TNFR-II |
| TNS1 | Tensin 1 | tensin, MST091, MST122, MST127, MSTP091, |
| | | MSTP122, MSTP127, PPP1R155, tensin-1, MXRA6, TNS |
| TP53 | Tumor Protein P53 | TRP53, BCC7, LFS1, P53 |
| TSPAN5 | Tetraspanin 5 | NET-4, TSPAN-5, tetraspanin-5, TM4SF9, NET4 |
| TXNRD1 | Thioredoxin Reductase 1 | TR1, TR, TRXR1, oxidoreductase, TXNR, GRIM-12, |
| | | GRIM12, KDRF |
| UBE2C | Ubiquitin-Conjugating Enzyme E2C | dJ447F3.2, UBCH10 |
| VEGFA | Vascular Endothelial Growth Factor | MVCD1, VEGF, VPF, VEGF-A |
| | A | |
| XK | X-Linked Kx Blood Group | NAC, neuroacanthocytosis, neurocanthocytosis, KX, |
| | | X1k, NA, MCLDS, XKR1, XRG1 |
| ZBTB10 | Zinc Finger And BTB Domain | RINZF, RINZFC |
| | Containing 10 | |

### Mathematical Models

A mathematical model can be used to determine the likelihood score for an immune-related adverse event associated with immunotherapy.

Various types of mathematical models may be used, including, e.g., the regression model in the form of logistic regression, principal component analysis, linear discriminant analysis, correlated component analysis, etc. These models can be used in connection with data from different sets of genes. The model for a given set of genes is applied to a training dataset, generating relevant parameters for a classifier. In some cases, these models with relevant parameters for a classifier can be applied back to the training dataset, or applied to a validation (or test) dataset to evaluate the classifier.

To apply the classifier to a test subject, a sample is collected from the test subject at a point in time after the subject has begun the immunotherapy treatment. In some embodiments, the sample is collected about 15 to 30 days after the immunotherapy treatment has begun. The levels of the selected biomarkers (representing expression of each of the genes in the gene set) in the sample are determined. These data are then tested in accordance with the classifier, and the subject's likelihood score for an immune-related adverse event (e.g., the probability that the immunotherapy will induce or at least be associated with an immune-related adverse event, or a value indicative of the probability that the immunotherapy will induce or be associated with an immune-related adverse event) is calculated.

As the immunotherapy involves gradual stimulation of the immune system, there is often a lag period before any adverse reactions can be observed. Thus, the classifier can offer an early determination regarding whether the immunotherapy treatment will induce a severe immune-related adverse event. Based on that determination, a physician can determine an appropriate treatment regimen for the subject. If the immunotherapy treatment is determined to be likely to cause a severe adverse reaction in the tested subject, the subject should be closely and actively monitored for early signs of even mild gastrointestinal effects. Instead or in addition, medical interventions (e.g., preventative anti-diarrhea medicine, immunosuppressant drugs, and/or lowered dose of immunotherapy) can be performed early in the course of therapy, before the subject's gastrointestinal condition would appear to call for them, as a prophylactic measure to prevent development of the predicted severe adverse event. In some cases, e.g., where the patient is so fragile that the risk of severe diarrhea is too great to take, a determination that the patient is at increased risk of severe diarrhea can lead to a recommendation to terminate the immunotherapy treatment for that subject, substituting another anti-cancer therapy less likely to trigger severe diarrhea. If appropriate, a different immunotherapy can be selected for treating cancer in the subject. In some cases, non-immunotherapy treatment should be recommended.

Some exemplary mathematic models are listed below.

### (1) Core Models/Core Classifiers

A “Core model” is a mathematical model that includes a core model gene set. Various types of mathematical models may be used as the core model, including, e.g., the regression model in the form of logistic regression, principal component analysis, linear discriminant analysis, and correlated component analysis etc.

The gene set for the Core models includes both CCR3 and PTGS2.

The gene set can be used in connection with a mathematical model, for example, logistic regression, to construct a Core model. The Core model can then be applied to a training dataset, generating appropriate classifier parameters, thus creating a “Core classifier.”

The classifier can be used to determine the likelihood score for an immune-related adverse event associated with immunotherapy. In some embodiments, the likelihood score indicates the probability that an immunotherapy will induce or otherwise be associated with an immune-related adverse event.

In some embodiments, the immune-related adverse event is diarrhea or colitis, which may present together.

In some embodiments, the immune-related adverse event is defined as any of a group of adverse reactions, such as Grade 3 and Grade 4 diarrhea. In such embodiments, if the immunotherapy induces, or otherwise is associated with, Grade 3 or Grade 4 diarrhea in a subject, the subject will be classified as having an immune-related adverse event, but if the immunotherapy induces only Grade 1 or Grade 2 diarrhea in the subject, or does not cause diarrhea in the subject, the subject will not be classified as having an immune-related adverse event. In some embodiments, the immune-related adverse event refers to a group of adverse reactions including not only Grade 3 and Grade 4 diarrhea, but also colitis.

In some embodiments, the immune-related adverse event refers specifically to a group of adverse reactions that includes Grade 2, Grade 3 and Grade 4 diarrhea. In such embodiments, if the immunotherapy induces, or otherwise is associated with, Grade 2, Grade 3, or Grade 4 diarrhea in a subject, the subject will be classified as having an immune-related adverse event, but if the immunotherapy induces Grade 1 diarrhea but no higher grade of diarrhea in the subject, or does not cause diarrhea in the subject, the subject will not be classified as having an immune-related adverse event. In some embodiments, the immune-related adverse event refers to a group of adverse reactions including not only Grade 2, Grade 3, and Grade 4 diarrhea, but also colitis.

### (2) Models and Classifiers for Classifying a Subject in Either (1) the Grade 3-4 Diarrhea/Colitis Group, or (2) the Grade 0-2 Diarrhea Group

In some embodiments, the immune-related adverse event is defined as any of a group of adverse events that includes only Grade 3 and Grade 4 diarrhea. Thus, the classifier determines a likelihood score indicating whether the gene-specific levels of mRNA transcribed from each gene of a defined set of genes in a blood sample from the test subject classifies with (A) a set of immunotherapy-intolerance levels, the set of immunotherapy-intolerance levels being a set of gene-specific levels of mRNA transcribed from each gene of the set of genes in blood samples collected from a first group of individuals who were treated with the immunotherapy prior to collecting the sample, wherein each individual of the first group experienced Grade 3 or Grade 4 diarrhea at some point during immunotherapy; as opposed to classifying with (B) a set of immunotherapy-tolerance levels, the set of immunotherapy-tolerance levels being a set of gene-specific levels of mRNA transcribed from each gene of the set of genes in blood samples collected from a second group of individuals who were treated with the immunotherapy prior to collecting the sample, wherein no individual of the second group experienced Grade 3 or Grade 4 diarrhea at any point during immunotherapy.

In some embodiments, the immune-related adverse event refers to a group of adverse reactions including only Grade 3 diarrhea, Grade 4 diarrhea, and colitis.

In some embodiments, the gene set for models for classifying a subject in either (1) the Grade 3-4 diarrhea group, or (2) the Grade 0-2 diarrhea group, includes both CCR3 and PTGS2. In some embodiments, the gene set for models for classifying a subject in either (1) the Grade 3-4 diarrhea/colitis group, or (2) the Grade 0-2 diarrhea group, includes both CCR3 and PTGS2.

In some embodiments, the gene set includes CCR3, MMP9, and PTGS2.

In some embodiments, the gene set further includes at least one gene, at least two genes, at least three genes, at least four genes, or all five genes selected from the group consisting of CARD12, CCND1, IL5, F5 and GYPA.

The gene set can be used in connection with a mathematical model, for example, logistic regression, to construct a model. The model can then be applied to a training dataset, generating appropriate classifier parameters.

### (3) Models and Classifiers for Classifying a Subject in Either (1) the Grade 2-4 Diarrhea/Colitis Group, or (2) the Grade 0-1 Diarrhea Group

In some embodiments, the immune-related adverse event is defined as any of a group of adverse reactions that includes only Grade 2, Grade 3 and Grade 4 diarrhea. Thus, the classifier determines a likelihood score indicating whether the gene-specific levels of mRNA transcribed from each gene of a defined set of genes in a blood sample from the test subject classifies with (A) a set of immunotherapy-intolerance levels, the set of immunotherapy-intolerance levels being a set of gene-specific levels of mRNA transcribed from each gene of the set of genes in blood samples collected from a first group of individuals who were treated with the immunotherapy prior to collecting the sample, wherein each individual of the first group experienced Grade 2, Grade 3, or Grade 4 diarrhea at some point during the immunotherapy; as opposed to classifying with (B) a set of immunotherapy-tolerance levels, the set of immunotherapy-tolerance levels being a set of gene-specific levels of mRNA transcribed from each gene of the set of genes in blood samples collected from a second group of individuals who were treated with the immunotherapy prior to collecting the sample, wherein no individual of the second group experienced Grade 2, Grade 3, or Grade 4 diarrhea at any point during immunotherapy.

In some embodiments, the immune-related adverse event is defined as any of a group of adverse reactions including Grade 2, Grade 3, and Grade 4 diarrhea, and colitis.

In some embodiments, the gene set for models for classifying a subject in either (1) the Grade 2-4 diarrhea, or (2) the Grade 0-1 diarrhea group includes both CCR3 and PTGS2. In some embodiments, the gene set for models for classifying a subject in either (1) the Grade 2-4 diarrhea/colitis group, or (2) the Grade 0-1 diarrhea group includes both CCR3 and PTGS2.

In some embodiments, the gene set includes CCL3, CCR3, IL8, and PTGS2. In some embodiments, the gene set further includes at least one gene, at least two genes, at least three genes, at least four genes, at least five genes, or all six genes selected from the group consisting of CARD12, F5, MMP9, SOCS3, IL5, and TLR9.

In some embodiments, the gene set includes CCL3, CCR3, IL8, and PTGS2, and further includes at least one gene, at least two genes, at least three genes, at least four genes, at least five genes, at least six genes, at least seven genes, at least eight genes, at least nine genes, at least ten genes, at least eleven genes, at least twelve genes, at least thirteen genes, at least fourteen genes, at least fifteen genes, or all sixteen genes selected from the group consisting of CARD12, CDC25A, CXCL1, F5, FAM210, GADD45A, IL18BP, IL2RA, IL5, IRAK3, ITGA4, MAPK14, MMP9, SOCS3, TLR9, and UBE2C.

In some embodiments, the gene set includes CARD12, CXCL1, F5, FAM210, GADD45A, IL18BP, IL2RA, IL5, IRAK3, ITGA4, MAPK14, MMP9, SOCS3, TLR9, and UBE2C.

The gene set can be used in connection with a mathematical model, for example, logistic regression, to construct a model. The model can then be applied to a training dataset, generating appropriate classifier parameters.

(4) Models and Classifiers for Classifying a Subject Who has Diarrhea into Either (1) the Grade 2-4 Diarrhea/Colitis Group, or (2) the Grade 1 Diarrhea Group

In some embodiments, the models and classifiers described herein can be used to classify a subject in either (1) the Grade 2-4 diarrhea/colitis group, or (2) the Grade 1 diarrhea group. In these cases, the subject has some mild symptoms of diarrhea (qualifying as Grade 1), but it is unknown whether the diarrhea is likely to progress to Grade 2 or higher. Thus, there is a need to quickly determine the likelihood that the subject will develop Grade 2-4 diarrhea and/or colitis during the course of the immunotherapy treatment. If the subject is determined to be likely to develop Grade 2-4 diarrhea and/or colitis (e.g., Grade 3 or Grade 4 diarrhea), the subject can be treated with any appropriate treatment for Grade 2-4 diarrhea and/or colitis (e.g., a steroid) as a prophylactic measure even before showing symptoms of Grade 2-4 diarrhea or colitis.

The classifier can determine a likelihood score indicating whether the gene-specific levels of mRNA transcribed from each gene of a defined set of genes in a blood sample from the test subject classifies with (A) a set of immunotherapy-intolerance levels, the set of immunotherapy-intolerance levels being a set of gene-specific levels of mRNA transcribed from each gene of the set of genes in blood samples collected from a first group of individuals who were treated with the immunotherapy prior to collecting the sample, wherein each individual of the first group experienced Grade 2, Grade 3, or Grade 4 diarrhea at some point during the immunotherapy; as opposed to classifying with (B) a set of immunotherapy-tolerance levels, the set of immunotherapy-tolerance levels being a set of gene-specific levels of mRNA transcribed from each gene of the set of genes in blood samples collected from a second group of individuals who were treated with the immunotherapy prior to collecting the sample, wherein each individual in the second group experienced Grade 1 diarrhea, but no higher grade of diarrhea (i.e., the most severe diarrhea each individual experienced is Grade 1 diarrhea) at any point during immunotherapy.

In some embodiments, the gene set for models for classifying a subject in either (1) the Grade 2-4 diarrhea group, or (2) the Grade 1 diarrhea group includes both CCR3 and PTGS2. In some embodiments, the gene set for models for classifying a subject in either (1) the Grade 2-4 diarrhea/colitis group, or (2) the Grade 1 diarrhea group includes both CCR3 and PTGS2.

In some embodiments, the gene set includes CCL3, CCR3, IL8, and PTGS2. In some embodiments, the gene set further includes at least one gene, at least two genes, at least three genes, at least four genes, at least five genes, or all six genes selected from the group consisting of CARD12, F5, MMP9, SOCS3, IL5, and TLR9.

In some embodiments, the gene set includes CCL3, CCR3, IL8, and PTGS2, and further includes at least one gene, at least two genes, at least three genes, at least four genes, at least five genes, at least six genes, at least seven genes, at least eight genes, at least nine genes, at least ten genes, at least eleven genes, at least twelve genes, at least thirteen genes, at least fourteen genes, at least fifteen genes, or all sixteen genes selected from the group consisting of CARD12, CDC25A, CXCL1, F5, FAM210, GADD45A, IL18BP, IL2RA, IL5, IRAK3, ITGA4, MAPK14, MMP9, SOCS3, TLR9, and UBE2C.

In some embodiments, the gene set includes CARD12, CXCL1, F5, FAM210, GADD45A, IL18BP, IL2RA, IL5, IRAK3, ITGA4, MAPK14, MMP9, SOCS3, TLR9, and UBE2C, as well as CCL3, CCR3, IL8, and PTGS2.

The gene set can be used in connection with a mathematical model, for example, logistic regression, to construct a model. The model can then be applied to a training dataset, generating appropriate classifier parameters.

### Classifiers

Referring to FIG. 1, classifiers are generated via data processing system 18 by applying one or more mathematical models to data representative of the level of transcribed mRNAs of selected genes across a population encompassing both immunotherapy subjects who experienced an immune-related adverse event or severe immune-related adverse event and subjects who experienced less severe immune-related adverse events or no immune-related adverse events.

In some embodiments, the mathematical model is logistic regression, as described herein. In these embodiments, data processing system 18 generates the classifier by applying the mathematical model with a set of genes to the training dataset to determine values for logistic regression equation coefficients and logistic regression equation constants. Generally, the training data set includes data representing levels of mRNA corresponding to one or more genes expressed in samples obtained from individuals of a training population (e.g., individuals who were administered a particular immunotherapy and did not experience diarrhea or colitis, experienced Grade 1-4 diarrhea with or without colitis, or had colitis without diarrhea). As described above, data processing system 18 generates and trains a classifier for each gene set. The classifier, which includes the mathematical model and the determined values of logistic regression equation coefficients and logistic regression equation constants, can be used to determine a likelihood score indicating a probability that immunotherapy will cause an immune-related adverse event in a test subject. Data processing system 18 then applies one or more of these generated classifiers to data specifying the level of mRNA expression corresponding to one or more of the genes of the gene set in a sample from the test subject, to determine a likelihood score indicating a probability that immunotherapy will cause an immune-related adverse event.

In some embodiments, the set of genes is selected based on the rule disclosed herein. In other embodiments, an individual gene is selected based on the p value as a measure of the likelihood that the transcribed mRNA of the individual gene can distinguish between the two phenotypic trait subgroups (i.e., subjects who experienced a specific immune-related adverse event vs. subjects who did not experience the specific immune-related adverse event). Thus, in some embodiments, genes are chosen to test in combination by input into a model wherein the p value of each gene is less than 0.2, less than 0.1, less than 0.5, less than 0.1, less than 0.05, less than 0.01, less than 0.005, less than 0.001, less than 0.0005, less than 0.0001, less than 0.00005, less than 0.00001, less than 0.000005, less than 0.000001, etc.

Classifiers can be used alone or in combination with each other to create a formula for determining the probability that a test subject will experience an immune-related adverse event associated with immunotherapy treatment. One or more selected classifiers can be used to generate a formula. It is not necessary that the method used to generate the data for creating the formulas be the same method used to generate data from the test subject.

In some embodiments, the individuals of the training population used to derive the model are different from the individuals of a population used to test the model. As would be understood by a person skilled in the art, this allows a person skilled in the art to characterize an individual whose phenotypic trait characterization is unknown, for example, to determine a likelihood score indicating the probability of that individual's experiencing an immune-related adverse event resulting from immunotherapy treatment, before the individual has experienced any symptoms indicative of the adverse event.

The data that is input into the mathematical model can be any data that is representative of the expression level of transcribed mRNA. Mathematical models useful in accordance with the disclosure include those using both supervised and unsupervised learning techniques. In one embodiment, the mathematical model chosen uses supervised learning in conjunction with a training population to evaluate each possible combination of transcribed mRNAs. Various mathematical models can be used, for example, a regression model, a logistic regression model, a neural network, a clustering model, principal component analysis, nearest neighbor classifier analysis, linear discriminant analysis, quadratic discriminant analysis, a support vector machine, a decision tree, a genetic algorithm, classifier optimization using bagging, classifier optimization using boosting, classifier optimization using the Random Subspace Method, a projection pursuit, and genetic programming and weighted voting, etc.

Applying a mathematical model to the data will generate one or more classifiers. In some embodiments, multiple classifiers are created that are satisfactory for the given purpose (e.g., all have sufficient AUC and/or sensitivity and/or specificity). In some embodiments, a formula is generated that utilizes more than one classifier. For example, a formula can be generated that utilizes classifiers in series. Other possible combinations and weightings of classifiers would be understood and are encompassed herein.

A classifier can be evaluated for its ability to properly characterize each individual of a population (e.g., a training population or a validation population) using methods known to a person of ordinary skill in the art. Various statistical criteria can be used, for example, area under the curve (AUC), sensitivity and/or specificity. In one embodiment, the classifier is evaluated by cross validation, Leave One OUT Cross Validation (LOOCV), n-fold cross validation, and jackknife analysis. In another embodiment, each classifier is evaluated for its ability to properly characterize those individuals of an immunotherapy-treated population not used to generate the classifier.

In some embodiments, the method used to evaluate the classifier for its ability to properly characterize each individual of the training population is a method that evaluates the classifier's sensitivity (true positive fraction) and 1-specificity (true negative fraction). In one embodiment, the method used to test the classifier is a Receiver Operating Characteristic (ROC), which provides several parameters to evaluate both the sensitivity and the specificity of the result of the equation generated. In one embodiment, the ROC area (area under the curve) is used to evaluate the equations. A ROC area greater than 0.5, 0.6, 0.7, 0.8, or 0.9 is preferred. A perfect ROC area score of 1.0 is indicative of both 100% sensitivity and 100% specificity. In some embodiments, classifiers are selected on the basis of the score. In an example, the scoring system used is a ROC curve score determined by the area under the ROC curve. In this example, classifiers with scores of greater than 0.95, 0.9, 0.85, 0.8, 0.7, 0.65, 0.6, 0.55, or 0.5 are chosen. In other embodiments, where specificity is important to the use of the classifier, a sensitivity threshold can be set, and classifiers ranked on the basis of the specificity are chosen. For example, classifiers with a cutoff for specificity of greater than 0.95, 0.9, 0.85, 0.8, 0.7, 0.65, 0.6, 0.55, 0.5 or 0.45 can be chosen. Similarly, the specificity threshold can be set, and classifiers ranked on the basis of sensitivity (e.g., greater than 0.95, 0.9, 0.85, 0.8, 0.7, 0.65, 0.6, 0.55, 0.5 or 0.45) can be chosen. Thus, in some embodiments, only the top ten ranking classifiers, the top twenty ranking classifiers, or the top one hundred ranking classifiers are selected. The ROC curve can be calculated by various statistical tools, including but not limited to Statistical Analysis System (SAS), CORExpress® statistical analysis software, and a web based calculator for ROC curves provided by Department of Radiology and Radiological Science, Johns Hopkins University School of Medicine, at a webpage located at World Wide Web (rad.jhmi.edu/jeng/javarad/roc/JROCFITi.html).

As would be understood by a person of ordinary skill in the art, the utility of the combinations and classifiers determined by a mathematical model will depend upon some characteristics (e.g., race, age group, gender, medical history) of the population used to generate the data for input into the model. One can select the individually identified genes or subsets of the individually identified genes, and test all possible combinations of the selected genes to identify useful combinations of gene sets.

Populations for Input into the Mathematical Models

Populations used for input should be chosen so as to result in a statistically significant classifier. In some embodiments, the reference or training population includes between 50 and 100 subjects. In another embodiment, the reference population includes between 100 and 500 subjects. In still other embodiments, the reference population includes two or more populations, each including between 50 and 100, between 100 and 500, between 500 and 1000, or more than 1000 subjects. The reference population includes two or more subpopulations. In one embodiment, the phenotypic trait characteristics of the two or more subpopulations are similar but for the phenotypic trait that is under investigation, for example, an immune-related adverse event associated with an immunotherapy. In some embodiments, the subpopulations are of roughly equivalent numbers. The present methods do not require using data from every member of a population, but instead may rely on data from a subset of a population in question.

For a reference population used to provide input into a mathematical model to identify those biomarkers that are useful in determining the probability that a subject will experience an adverse reaction to immunotherapy treatment, the reference population includes individuals who experienced a particular immune-related adverse event associated with an immunotherapy (e.g., a severe immune-related adverse event of one particular type (e.g., diarrhea), or of any of a set of types immune-related adverse events attributable to the immunotherapy) and individuals who did not experience the particular immune-related adverse event. The latter group may have experienced instead a moderate, mild or no immune-related adverse event, or an immune-related adverse event of a type different from the particular type.

In some embodiments, a test population (or a validation population), which is comprised of individuals who experienced an immune-related adverse event and individuals who did not experience the immune-related adverse event, is used to evaluate a classifier for its ability to properly characterize each individual.

Data for Input into the Mathematical Models

Data for input into the mathematical models are data representative of the respective levels of products of a set of genes. In one embodiment, the data are a measure that represents a gene-specific level of transcribed RNA from a gene of a set of genes. The RNA includes, but is not limited to, mRNA, all spliced variants of the mRNA, and unspliced transcript. In another embodiment, all of the RNA products are expressed in blood. In some embodiments, the data are a measure that represents a gene-specific level of protein. The level of a protein can be determined by any techniques that are known in the art, for example, protein mass spectrometry and enzyme-linked immunosorbent assay (ELISA).

A dataset can be used to generate a classifier. The “dataset,” in the context of a dataset to be applied to a classifier, can include data representing levels of each biomarker for each individual. However, in some embodiments, the dataset does not need to include data for each biomarker of each individual. For example, the data set includes data representing levels of each biomarker for fewer than all of the individuals (e.g., 99%, 95%, 90%, 85%, 80%, 75%, 70% or fewer) and can still be useful for purposes of generating a classifier.

### Mathematic Model Forms

In some embodiments, a mathematic model has the form:

V=α+Σβᵢƒ(Xᵢ)

In this form of the model, V is a value indicating the probability that the immunotherapy will cause an immune-related adverse event. In some embodiments, the immune-related adverse event is a severe adverse reaction to immunotherapy treatment. In some embodiments, the immune-related adverse event is Grade 3 or Grade 4 diarrhea. In some embodiments, the immune-related adverse event is Grade 2, Grade 3 or Grade 4 diarrhea. In some embodiments, the immune-related adverse event is colitis.

Xᵢ represents the level of mRNA transcribed from an ith gene of the set of genes in a sample from the test subject. βᵢ is a coefficient for ƒ(Xᵢ), which is a variable corresponding to the level of mRNA transcribed from the ith gene. The function ƒ(x) is a function that gives a corresponding value of x. In one embodiment, ƒ(x)=x. Thus, the mathematic model can have the form V=α+Σβᵢ Xᵢ. In some other embodiments, ƒ(x) may be a function for normalization or standardization. In a variation, the formula may include additional parameters to account for age, sex, and race category.

In some embodiments, V is an actual probability (a number varying between 0 and 1). In other embodiments, V is a value from which a probability can be derived.

In some embodiments, the mathematical model is a regression model, for example, a logistic regression model or a linear regression model. The regression model can be used to test various sets of genes.

In the case of linear regression models, the classifiers generated can be used to analyze expression data from a test subject and to provide a result indicative of a quantitative measure of the test subject, for example, the likelihood score for an immune-related adverse event associated with an immunotherapy.

In general, a linear regression equation is expressed as

Y=α+β₁X₁+β₂X₂+ . . . +βₖXₖ+ε

Y, the dependent variable, indicates a quantitative measure of a biological feature (e.g., a likelihood score for an immune-related adverse event associated with an immunotherapy).

The dependent variable Y depends on k explanatory variables (the measured characteristic values for the k select genes, e.g., the level of transcribed mRNA from subjects in the first and second subgroups), plus an error term that encompasses various unspecified omitted factors. In the above-identified model, the parameter β₁ gauges the effect of the first explanatory variable X₁ on the dependent variable Y. β₂ gives the effect of the explanatory variable X₂ on Y.

A logistic regression model is a non-linear transformation of the linear regression. The logistic regression model is often referred to as the “log it” model and can be expressed as

ln[p/(1−p)]=α+βᵢX₁+β₂X₂+ . . . +βₖXₖ+ε

where,

α and ε are constants

ln is the natural logarithm, log₍ₑ₎, where e=2.71828 . . . ,

p is the probability that the event Y occurs, p(Y=1),

p/(1−p) is the “odds ratio,”

In [p/(1−p)] is the log odds ratio, or “log it”.

It will be appreciated by those of skill in the art that a and c can be folded into a single constant, and expressed as a. In some embodiments, a single term a is used, and c is omitted. The “logistic” distribution is an S-shaped distribution function. The log it distribution constrains the estimated probabilities (p) to lie between 0 and 1.

In some embodiments, the logistic regression model is expressed as

Y=α+ΣβᵢXᵢ

Here, Y is a value indicating a probability that the set of test levels classifies with the set of immunotherapy-intolerance levels, as opposed to the set of immunotherapy-tolerance levels. In some embodiments, the set of immunotherapy-intolerance levels is a set of gene-specific levels of mRNA transcribed from each gene of the set of genes in blood samples collected from a group of individuals who were treated with the immunotherapy prior to collecting the sample, wherein each individual of the group experienced the immune-related adverse event during the course of receiving the immunotherapy. In some embodiments, the set of immunotherapy-tolerance levels is a set of gene-specific levels of mRNA transcribed from each gene of the set of genes in blood samples collected from a second group of individuals who were treated with the immunotherapy prior to collecting the sample, wherein each individual of the second group did not experience the immune-related adverse event during the course of receiving the immunotherapy.

Xi is a level of mRNA transcribed from an ith gene of the set of genes in blood of the test subject, βi is a logistic regression equation coefficient for the ith gene, α is a logistic regression equation constant that can be zero, and βi and α are the result of applying logistic regression analysis to the set of immunotherapy-intolerance levels and the set of immunotherapy-tolerance levels.

In some embodiments, the logistic regression model is fit by maximum likelihood estimation (MLE). The coefficients (e.g., α, β1, β2, . . . ) are determined by maximum likelihood. A likelihood is a conditional probability (e.g., P(Y|X), the probability of Y given X). The likelihood function (L) measures the probability of observing the particular set of dependent variable values (Y1, Y2, . . . , Yn) that occur in the sample data set. In some embodiments, it is written as the product of the probability of observing Y1, Y2, . . . , Yn:

L=Prob(Y1,Y2, . . . ,Yn)=Prob(Y1)*Prob(Y2)* . . . Prob(Yn)

The higher the likelihood function, the higher the probability of observing the Ys in the sample. MLE involves finding the coefficients (α, β1, β2, . . . ) that make the log of the likelihood function (LL<0) as large as possible or −2 times the log of the likelihood function (−2LL) as small as possible. In MLE, some initial estimates of the parameters α, β1, β2, and so forth are made. Then, the likelihood of the data given these parameter estimates is computed. The parameter estimates are improved, and the likelihood of the data is recalculated. This process is repeated until the parameter estimates remain substantially unchanged (for example, a change of less than 0.01 or 0.001). Examples of logistic regression and fitting logistic regression models are found in Hastie, The Elements of Statistical Learning, Springer, N.Y., 2001, pp. 95-100.

Once the logistic regression equation coefficients and the logistic regression equation constant are determined, the classifier can be readily applied to a test subject to obtain Y. In one embodiment, Y can be used to calculate probability (p) by solving the function Y=In (p/(1−p)), using data process system 18.

In some embodiments, explanatory variables are standardized before fitting into the model. Standardized coefficients (or beta coefficients) are the estimates resulting from a regression analysis that have been standardized so that the variances of dependent and explanatory variables are 1. Therefore, standardized coefficients represent how many standard deviations a dependent variable will change, per standard deviation increase in the explanatory variable. For univariate regression, the absolute value of the standardized coefficient equals the correlation coefficient. Standardization of the coefficient is usually performed to identify which of the explanatory variables have a greater effect on the dependent variable in a multiple regression analysis. In one embodiment, variables are standardized before fitting into a logistic regression model. Standardized logistic regression coefficients (or standardized beta coefficients) are the estimates resulting from performing a logistic regression analysis on variables that have been standardized. In some embodiments, only explanatory variables are standardized, and in some other embodiments, only dependent variables are standardized. Further, in some embodiments, both explanatory variables and dependent variables are standardized. In one embodiment, the standardized regression coefficient equals the corresponding unstandardized coefficient multiplied by the ratio std(Xᵢ)/std(Y), where “std” denotes standard deviation.

### Other Mathematical Models

The statistical techniques described above are examples of the types of models that can be used to construct classifiers useful to determine whether a subject is relatively likely to experience an immune-related adverse event associated with an immunotherapy. There are various types of classifiers, including, e.g., clustering, principal component analysis, nearest neighbor classifier analysis, linear discriminant analysis, and support vector machines.

### Rounding and Range

Rounding refers to a mathematical operation that replaces a value by another value that is approximately equal but has a shorter, simpler, or more explicit representation. The most common type of rounding is to round to an integer; or, more generally, to an integer multiple of some increment, for example, tenths, hundredths, or five tenths. When rounding to a predetermined number of significant digits, the increment m depends on the magnitude of the number to be rounded (or of the rounded result). The increment m is normally a finite fraction in a number system that is used to represent the numbers. For example, in the decimal number system, m is an integer times a power of 10, such as 1×10⁻³ or 25×10⁻². The experimentally-derived value provided in the examples and tables of the present disclosure for each coefficient or constant has n significant digits after the decimal point. Each value can be rounded to n−1 or n−2 or n−3 significant digits. Thus, a number shown with n significant digits after the decimal point is intended to provide literal support for the same number that is rounded to a number with fewer significant digits after the decimal point (e.g., n−1, n−2, n−3). For example, the number “−0.7709” (with four significant digits after the decimal point) is intended to provide full literal support for expressing the same number as −0.771, −0.77, −0.8, or −1. Similarly, the number “0.1132” is intended to provide full literal support for expressing the same number as 0.113, 0.11, 0.1, or 0.

It is also recognized by a person skilled in the art that the experimentally-derived value provided in the examples and tables of the present disclosure for each coefficient or constant in each model can be increased or decreased by an appropriate amount (e.g., 50%, 30%, 25%, 20%, 10%, or 5%) and still produce models useful in the data processing methods described in this disclosure. Thus, the value for each coefficient and constant listed in any of the tables explicitly constitutes a disclosure not only of that precise value, but also each of the following specific ranges surrounding that value: +/−50%, +/−30%, +/−25%, +/−20%, +/−10%, and +/−5%. For example, a coefficient listed in a table as “−0.2932” is deemed to be a disclosure not only of −0.2932 per se (and, when that number is rounded off, a disclosure of −0.293, and −0.29, and −0.3), but also a disclosure of “−0.2932+/−50%”, corresponding to a range of −0.4395 to −0.1465; and a disclosure of “−0.2932+/−30%”, corresponding to a range of −0.3812 to −0.2052; and a disclosure of “−0.2932+/−25%”, corresponding to a range of −0.3665 to −0.2199; and a disclosure of “−0.2932+/−20%”, corresponding to a range of −0.3518 to −0.2346; and a disclosure of “−0.2932+/−10%”, corresponding to a range of −0.3225 to −0.2639; and a disclosure of “−0.2932+/−5%”, corresponding to a range of −0.3079 to −0.2785.

Furthermore, as each coefficient or constant in each model can be increased or decreased by an appropriate amount and still remain useful in the present methods, the value for each coefficient and constant listed in any of the tables also explicitly constitutes a disclosure for a value that is reasonably close to the explicitly disclosed value. For example, a constant listed in a table as “−28.231” is deemed to be a disclosure not only of −28.231 per se (and a disclosure of rounded-off versions of that number, including −28.23, and −28.2, and −28), but also a disclosure of “about −28.231”, “about −28.23”, “about −28.2”, and “about −28.”

### Kits

The gene-specific levels of RNAs transcribed from a set of genes can be determined by using a kit. Such a kit can include materials and reagents required for obtaining an appropriate blood sample from a subject, or for measuring the levels of particular transcribed RNAs. In some embodiments, a kit includes primers appropriate for the transcribed RNAs.

In another embodiment, a kit is designed to determine the amounts of particular proteins present in a sample. The amount of a protein can be determined by any techniques that are known in the art, for example, protein mass spectrometry and enzyme-linked immunosorbent assay (ELISA). The kit includes materials and reagents required for measuring the amount of protein products of a particular set of genes, for example, an antibody or antibody fragment that targets each protein of interest.

In some embodiments, a kit may further include one or more reagents for various purposes, such as: (1) reagents for purifying RNA from blood; (2) primers for transcribed mRNA; (3) dNTPs and/or rNTPs (either premixed or separate), optionally with one or more uniquely labeled dNTPs and/or rNTPs (e.g., biotinylated or Cy3 or Cy5 tagged dNTPs); (4) post-synthesis labeling reagents, such as chemically active derivatives of fluorescent dyes; (5) enzymes, such as reverse transcriptases, DNA polymerases, and the like; (6) various buffer mediums, e.g., hybridization and washing buffers; (7) labeled probe purification reagents and components, e.g., spin columns; (8) protein purification reagents; and/or (9) signal generation and detection reagents, e.g., streptavidin-alkaline phosphatase conjugate, fluorescent or chemiluminescent substrate, and the like. In some embodiments, the kits may include pre-labeled protein or RNA transcript (for example, 18S RNA and (3-actin mRNA) for use as a control.

In some embodiments, the kits are Quantitative PCR (QPCR) kits. In other embodiments, the kits are nucleic acid arrays or protein arrays or antibody arrays. In one embodiment, kits for measuring an RNA product of a gene includes materials and reagents that are necessary for measuring the expression of the RNA product. For example, a microarray or a QPCR kit may contain only those reagents and materials that are necessary for measuring the levels of RNA products of a set of genes that are disclosed in the present disclosure. In some other embodiments, the kits can include materials and reagents for RNA products that are not discussed in the present disclosure.

For nucleic acid microarray kits, the kits generally include probes attached or localized to a support surface. The probes may be labeled with a detectable label. In one embodiment, the probes are specific for the 5′ region, the 3′ region, the internal coding region, an exon(s), an intron(s), an exon junction(s), or an exon-intron junction(s), of a RNA product(s). The microarray kits may include instructions for performing the assay and methods for interpreting and analyzing the data resulting from the performance of the assay. The kits may also include hybridization reagents and/or reagents necessary for detecting a signal when a probe hybridizes to a target nucleic acid sequence. Generally, the materials and reagents for the microarray kits are in one or more packages.

For QPCR kits, the kits generally include pre-selected primers specific for RNA products (e.g., an exon(s), an intron(s), an exon junction(s), and an exon-intron junction(s)). The QPCR kits may also include enzymes suitable for reverse transcribing and/or amplifying nucleic acids (e.g., polymerases such as Taq, reverse transcriptase etc.), and deoxynucleotides and buffers needed for the reaction mixture for reverse transcription and amplification. The probes may or may not be labeled with a detectable label (e.g., a fluorescent label). In some embodiments, when contemplating multiplexing, the probes are labeled with a different detectable label (e.g. carboxyfluorescein (FAM) or hexachloro-fluorescein (HEX)). These kits may include different containers suitable for each individual reagent, enzyme, primer and probe. Further, the QPCR kits may include instructions for performing the assay and methods for interpreting and analyzing the data resulting from the performance of the assay. The instructions for analyzing the data will typically be provided on a machine-readable medium programmed in accordance with the presently disclosed analytical methods. For antibody based kits, the kit can include, for example: (1) a first antibody (which may or may not be attached to a support) which binds to protein of interest (e.g., protein products of a set of genes); and, optionally, (2) a second, different antibody which binds to either the protein, or the first antibody and is conjugated to a detectable label (e.g., a fluorescent label, a radioactive isotope or an enzyme). The antibody-based kits may also include beads for conducting an immunoprecipitation. Each component of the antibody-based kits is generally in its own suitable package. Thus, these kits generally include different packages suitable for each antibody. Further, the antibody-based kits may include instructions for performing the assay and methods for interpreting and analyzing the data resulting from the performance of the assay. The instructions for analyzing the data will typically be provided on a machine-readable medium programmed in accordance with the presently disclosed analytical methods.

### EXAMPLES

The present disclosure further describes the following examples, which do not limit the scope of the present disclosure.

### Example 1: Patient Population

Two patient populations with melanoma were used for creating and evaluating classifiers. Detailed descriptions of the two patient populations can be found in US 2011/0070582; Kirkwood, John M., et al, “Phase II trial of tremelimumab (CP-675,206) in patients with advanced refractory or relapsed melanoma,” Clinical Cancer Research 16.3 (2010): 1042-1048; Ribas, Antoni, et al, “Phase III randomized clinical trial comparing tremelimumab with standard-of-care chemotherapy in patients with advanced melanoma,” Journal of Clinical Oncology 31.5 (2013): 616-622; each of which is incorporated by reference in its entirety. Data derived from analysis of whole blood RNA transcripts of particular genes for the two patient populations have been deposited in the National Center for Biotechnology Information (NCBI) Gene Expression Omnibus (GEO) public data repository. The data are publicly accessible through GEO accession number GSE94873 (ncbi.nlm.nih.gov/geo/query/acc.cgi?acc=GSE94873).

### “1008” Patient Population (Training Dataset)

A worldwide Phase 2, multi-center, open-label, non-randomized, multi-national study of tremelimumab was carried out inpatients at disease stages IIIC, IV M1a, M1b and IV M1c. All patients previously received chemotherapy (See Kirkwood, John M., et al, “Phase II trial of tremelimumab (CP-675,206) in patients with advanced refractory or relapsed melanoma,” Clinical Cancer Research 16.3 (2010): 1042-1048).

The original patient population included 218 patients. However, whole blood samples were collected approximately 30 days following the start of tremelimumab treatment were obtained for only 150 of the 218 patients. The 150 patients are referred to here as the “1008 patient population” or “1008 training population.” (The dataset obtained from that patient population is termed the “1008 training dataset” or “1008 dataset.”)

All patients met the following inclusion criteria:
1) Histologically confirmed melanoma that was surgically incurable and either a) Stage III melanoma (AJCC 6th Edition) including locally relapsed, in transit lesions or draining nodes, or b) Stage IV melanoma (M1a, M1b, M1c);
2) Received prior treatment including at least one systemic therapy for treatment of metastatic disease (prior systemic regimen for the treatment of metastatic melanoma included IL-2, dacarbazine and/or temozolamide or interferon-α; patient have received at least one cycle at full dose);
3) Documented disease progression after the last dose of prior therapy (including patients whose disease progressed during previous treatment (refractory), recurred following previous treatment (relapsed) or patients who could not tolerate previous treatment due to unacceptable toxicity and subsequently progressed);
4) At least one measurable lesion according to Response Evaluation Criteria in Solid Tumors (RECIST) (where measurable disease is defined as at least one lesion that can be accurately measured in at least one dimension with longest diameter=2.0 cm using conventional techniques or =1.0 cm with spiral CT scan. Skin lesions documented by color photography have a longest diameter of at least 1.0 cm. If the measurable disease is restricted to a solitary lesion, its neoplastic nature is confirmed by cytology or histology. Clinically detected lesions will only be considered measurable when they are superficial (e.g., skin nodules) and the longest diameter is =2 cm. Palpable lymph nodes >2.0 cm should be demonstrable by CT scan. Tumor lesions that are situated in a previously irradiated area will be considered measurable if progression is documented following completion of radiation therapy);
5) ECOG performance status (PS) 0 or 1;
6) Age 18 years or older;
7) Adequate bone marrow, hepatic, and renal function determined within 14 days prior to enrollment, defined as: a) absolute neutrophil count=1.5×109 cells/L; b) platelets=100×109/L; c) hemoglobin=10 g/dL; d) aspartate and alanine aminotransferases (AST, ALT)=2.5×ULN(=5×ULN, if documented liver metastases are present); e) total bilirubin=2×ULN (except patients with documented Gilbert's syndrome); 0 serum creatinine=2.0 mg/dL or calculated creatinine clearance=60 mL/min;
8) Serum lactic acid dehydrogenase (LDH)=2×ULN;
9) Patients have recovered from prior treatment-related toxicities, to baseline status, or to NCI CTCAE (v 3.0) Grade of 0 or 1, except for toxicities not considered a safety risk such as alopecia or residual peripheral neuropathy resulting from prior systemic therapy. Post-surgical pain shall not be considered a basis for exclusion; and
10) Have been willing and able to provide written informed consent.

Any subjects that met any of the following criteria were excluded from the study:
1) Diagnosed with melanoma of ocular origin (e.g., uveal melanoma);
2) Received treatment for cancer, including immunotherapy, within one month prior to enrollment (e.g., dosing);
3) Received any prior vaccine therapy for the treatment of melanoma within the last 6 months (if received last dose of vaccine prior to 6 months, patient is eligible);
4) Received any prior CTLA4-inhibiting agent;
5) History of chronic autoimmune disease (e.g., Addison's disease, multiple sclerosis, Graves disease, Hashimoto's thyroiditis, inflammatory bowel disease, psoriasis, rheumatoid arthritis, systemic lupus erythematosus, hypophysitis, etc.; active vitiligo or a history of vitiligo will not be a basis for exclusion);
6) Known active or chronic viral hepatitis;
7) History of inflammatory bowel disease, celiac disease, or other chronic gastrointestinal conditions associated with diarrhea or current acute colitis of any origin;
8) History of uveitis or melanoma-associated retinopathy;
9) Potential requirement for systemic corticosteroids or concurrent immunosuppressive drugs based on prior history or received systemic steroids within the last 4 weeks prior to enrollment (note: inhaled or topical steroids in standard doses were allowed);
10) Dementia or significantly altered mental status that would prohibit the understanding or rendering of informed consent and compliance with the requirements of this protocol;
11) Any serious, uncontrolled medical disorder or active infection, which would impair their ability to receive study treatment. Patients with evidence of Acquired Immunodeficiency Syndrome (AIDS) are excluded;
12) Brain metastases, radiological documentation of absence of brain metastases at screening was required for patients (note that a history of treated brain metastases was acceptable);
13) History of other malignancies, except for adequately treated basal cell carcinoma or squamous cell skin cancer or carcinoma of cervix, unless the patient was disease-free for at least 5 years; and
14) Pregnancy or breast-feeding (female patients are surgically sterile or are postmenopausal for two years, or have agreed to use effective contraception during the period of treatment and 12 months after. Female patients with reproductive potential have had a negative pregnancy test (serum/urine) within 72 hours prior to enrollment).

An anti-CTLA4 treatment (tremelimumab) was administered intravenously at a dose of 15 mg/kg every 90 days in patients with previously treated advanced melanoma. Patients were allowed to receive up to 4 doses of Tremelimumab in a 12-month period. Tumor data were reviewed under the RECIST guidelines.

Blood samples were collected approximately 30 days following the start of the immunotherapy treatment for the 150 patients. Many of the 150 patients developed diarrhea during the 12-month treatment period. The most severe level of diarrhea experienced by each of the 150 subjects over the 12-month period is summarized as follows:
Grade 4 Diarrhea: 0 subjects (N=0)
Grade 3 Diarrhea: 9 subjects (N=9)
Grade 2 Diarrhea: 12 subjects (N=12)
Grade 1 Diarrhea: 39 subjects (N=39)
Grade 0 Diarrhea (i.e., no diarrhea): 90 subjects (N=90)

### “1009” Patient Population (Test or Validation Dataset)

A worldwide Phase 3, multi-national, open-label, 2-arm randomized study was carried out in 264 patients with unresectable metastatic melanoma who have received no prior chemotherapy, immunotherapy or biological therapy for the treatment of metastatic disease. The patients were at disease stages IIIC, IV M1a, IV M1b, and IV M1c (See, Ribas et al., “Phase III randomized clinical trial comparing tremelimumab with standard-of-care chemotherapy in patients with advanced melanoma,” Journal of Clinical Oncology 31.5 (2013): 616-622).

The overall study originally involved 264 patients. However, whole blood samples were collected approximately 30 days following the start of treatment in only 210 of the 264 patients. The 210 patients are referred to here as the “1009 patient population” or “1009 validation population.” (The dataset obtained from that patient population is termed the “1009 validation dataset” or “1009 dataset.”) All patients met the following inclusion criteria:
1) Histologically confirmed melanoma that is not surgically curable and is either: a) Stage IV (AJCC 6th edition) or b) Stage IIIC (AJCC 6th edition) with N3 status for regional lymph nodes and in-transit or satellite lesions (note: patients with mucosal melanoma were not excluded; HLA types were eligible);
2) Patients may have either had measurable disease or non-measurable disease which could be evaluated for objective response. A measure disease includes at least lesion that meets the following criteria: a sole lesion that can be accurately measured in at least one dimension, lesion on a CT scan has a longest diameter ≥2.0 cm using conventional techniques or ≥1.0 cm with spiral CT scan. A skin lesion has longest diameter at least 1.0 cm, clinically detected lesions are superficial (e.g., skin nodules), and the longest diameter is ≥2.0 cm, palpable lymph nodes ≥2.0 cm should be demonstrable by CT scan. If the measurable disease is restricted to a solitary lesion, its neoplastic nature is confirmed by cytology or histology. Tumor lesions that are situated in a previously irradiated area will be considered measurable if progression is documented following completion of radiation therapy. Non-measurable disease includes lesions that fail to meet the above criteria for measurability. Evidence of disease confirmed by pathology, i.e., needle aspirate/biopsy; patients with previously irradiated lesions have documented progression or disease outside the radiation port;
3) Eastern Cooperative Oncology Group (ECOG) performance status of 0 or 1;
4) Age 18 years or older;
5) Adequate bone marrow, hepatic, and renal function determined within 14 days prior to randomization, defined as: a) Absolute neutrophil count ≥1.5×109 cells/L; b) Platelets ≥100×109/L; c) Hemoglobin ≥10 g/dL; d) Aspartate and alanine aminotransferases (AST, ALT)≤2.5× Upper Limit of Normal (ULN), or ≤5×ULN, if documented liver metastases are present; e) Total serum bilirubin ≤1.5×ULN (except patients with documented Gilbert's syndrome); and f) Serum creatinine ≤2.0 mg/dL or calculated creatinine clearance ≥60 mL/min;
6) Serum lactic acid dehydrogenase (LDH)≤2×ULN;
7) CT scan of the brain with contrast or MRI of the brain within 28 days of enrollment showing no evidence of brain metastases;
8) Patients have recovered from prior surgical or adjuvant (alpha-interferon) treatment-related toxicities, to baseline status, or a CTC Grade of 0 or 1, except for toxicities not considered a safety risk, such as alopecia; post-surgical pain was not considered a basis for exclusion;
9) Females of childbearing potential have had a negative serum or urine pregnancy test within 14 days prior to randomization; females who underwent surgical sterilization or who were postmenopausal for at least 2 years were not considered to be of childbearing potential;
10) Females of childbearing potential and males who have not undergone surgical sterilization have agreed to practice a form of effective contraception prior to entry into the study and for 6 months following the last dose of study drug; and
11) Patients have been willing and able to provide written informed consent.

Any subjects that met any of the following criteria were excluded from the study:
1) Melanoma of ocular origin;
2) Received any systemic therapy for metastatic melanoma except post-surgical adjuvant treatment with alpha-interferon for resected Stage II or Stage III disease; patients who received alpha-interferon have been at least 30 days from the last dose, and have documented tumor progression since the last dose (prior chemotherapy, biochemotherapy, cytokine therapy (other than alpha-interferon), or vaccine therapy was not allowed; prior intralesional injections and prior isolated limb perfusion therapy were not allowed; prior resection for Stage III or Stage IV disease is allowed as long as the patient had unresectable lesions at the time of randomization);
3) History of brain metastases;
4) Received any prior CTLA4 inhibiting agent;
5) Patients previously randomized on this protocol;
6) History of chronic inflammatory or autoimmune disease (e.g., Addison's disease, multiple sclerosis, Graves' disease, Hashimoto's thyroiditis, psoriasis, rheumatoid arthritis, systemic lupus erythematosus, hypophysitis, pituitary disorders, etc.; active vitiligo or a history of vitiligo was not a basis for exclusion);
7) History of uveitis or melanoma-associated retinopathy;
8) History of inflammatory bowel disease, celiac disease, or other chronic gastrointestinal conditions associated with diarrhea or bleeding, or current acute colitis of any origin;
9) History of hepatitis due to Hepatitis B virus or Hepatitis C virus;
10) Any serious uncontrolled medical disorder or active infection that would impair the patient's ability to receive study treatment;
11) Received an immunosuppressive dose of corticosteroids or other immunosuppressive medication (e.g., methotrexate, rapamycin) within 30 days of randomization (patients with adrenal insufficiency could take up to 5 mg of prednisone or equivalent daily; topical and inhaled corticosteroids in standard doses were allowed);
12) History of other malignancy, except for adequately treated basal cell carcinoma or squamous cell skin cancer or carcinoma in situ of the cervix, unless the patient had been disease-free for at least 5 years;
13) Breast-feeding; and
14) Dementia or significantly altered mental status that would prohibit the understanding or rendering of informed consent and compliance with the requirements of this protocol.

Patients were randomized to receive intravenous administration of an anti-CTLA4 treatment (tremelimumab) at a dose of 15 mg/kg on Day 1 of every 90-day cycle, for up to four cycles. Tremelimumab at 15 mg/kg was administered by IV infusion once every 90 days for up to four cycles to patients. Tremelimumab mechanism of action involves stimulation of an immune response, and there is an expected lag period before an effective immune response is initiated. Therefore, patients with evidence of disease progression at the first tumor assessment were allowed to continue to receive tremelimumab if they did not have clinical signs or symptoms of progression. No dose reductions were permitted; however, dose delays were permitted to allow recovery from potential treatment-related toxicity. Patients randomly assigned to the standard-of-care arm received either single-agent dacarbazine (DTIC) (1,000 mg/m2) IV on day 1 of a 21-day cycle or single-agent temozolomide (200 mg/m2) orally on days 1 to 5 of a 28-day cycle. Choice of chemotherapeutic agent was at the discretion of the investigator. Chemotherapy was administered for up to 12 cycles or until disease progression, unacceptable toxicity, or withdrawal of consent. Dose reductions or delays were permitted. Crossover to the tremelimumab cohort was not allowed for patients who progressed after treatment with DTIC or temozolomide.

Tumor responses were assessed every 90 days (one cycle) in patients treated with tremelimumab, every 42 days (two cycles) in patients treated with DTIC, and every 56 days (two cycles) in patients treated with temozolomide. In both study arms, there was a planned assessment of tumor response at 6 months to determine PFS rate at this time point. Tumor data assessed by investigators were reviewed by the sponsor to ensure compliance with RECIST criteria. Patients were evaluated for toxicity at every scheduled visit, and any toxicities were assessed according to the National Cancer Institute Common Terminology Criteria for Adverse Events, version 3.0. A detailed description of this clinical trial can be found in, e.g., Ribas et al. “Phase III randomized clinical trial comparing tremelimumab with standard-of-care chemotherapy in patients with advanced melanoma.” Journal of Clinical Oncology 31.5 (2013): 616-622; and Saenger et al. “Blood mRNA expression profiling predicts survival in patients treated with tremelimumab.” Clinical Cancer Research 20.12 (2014): 3310-3318.

Blood samples were collected approximately 30 days following the start of treatment for the 210 subjects. Many of the 210 subjects developed diarrhea during the 12-month treatment period. The most severe level of diarrhea experienced by each of the 210 subjects over the 12-month period is summarized as follows:
Grade 4 Diarrhea: 1 subject (N=1)
Grade 3 Diarrhea: 26 subjects (N=26)
Grade 2 Diarrhea: 29 subjects (N=29)
Grade 1 Diarrhea: 36 subjects (N=36)
Grade 0 Diarrhea (i.e., no diarrhea): 118 subjects (N=118)

### Example 2: Sample Preparation

Whole blood samples were obtained from the patients approximately 30 days after the patients received the first dose of immunotherapy. RNA was isolated using the PAXgene™ Blood RNA System (Pre-Analytix). Quantitative PCR assays were performed using custom primers and probes for the 169 targeted genes shown in Table 2, to obtain gene expression measurements.

### RNA Extraction

Human blood was obtained by venipuncture in an mRNA stabilization collection tube and prepared for assay. Cells were lysed and nucleic acids purified. RNA was obtained from the nucleic acid mix using a filter-based RNA isolation system from Ambion (RNAqueous™, Phenol-free Total RNA Isolation Kit, Catalog #1912, version 9908; Austin, Tex.) and the PAXgene™ Blood RNA System (from Pre-Analytix).

cDNA Synthesis

cDNA was synthesized from each RNA sample.

### Materials:

### Applied Biosystems TAQMAN Reverse Transcription Reagents Kit (P/N 808-0234).

Kit Components: 10× TaqMan RT Buffer, 25 mM Magnesium chloride, deoxyNTPs mixture,

### Random Hexamers, RNase Inhibitor, MultiScribe Reverse Transcriptase (50 U/mL)

RNase/DNase free water (DEPC Treated Water from Ambion (P/N 9915G), or equivalent).

### Method:

1) RNase Inhibitor and MultiScribe Reverse Transcriptase were placed on ice. Other reagents were thawed at room temperature and then placed on ice.
2) RNA samples were removed from −80° C. freezer and thawed at room temperature and then placed immediately on ice.
3) The cocktail of Reverse Transcriptase Reagents was prepared for each 100 mL RT reaction (for multiple samples, extra cocktail was prepared to allow for pipetting error):
   1 reaction (mL) 11×, e.g., for 10 samples (μL):
   10× RT Buffer 10.0 110.0
   25 mM MgCl₂ 22.0 242.0
   dNTPs 20.0 220.0
   Random Hexamers 5.0 55.0
   RNAse Inhibitor 2.0 22.0
   Reverse Transcriptase 2.5 27.5
   Water 18.5 203.5
   Total: 80.0 880.0 (80 μL per sample)
4) Each RNA sample was brought to a total volume of 20 μL in a 1.5 mL microcentrifuge tube (10 μL RNA was diluted to 20 μL with RNase/DNase free water; for whole blood RNA, 20 μL total RNA was used). 80 μL RT reaction mix was added to RNA sample and mixed by pipetting up and down.
5) Samples were incubated at room temperature for 10 minutes, and then at 37° C. for 1 hour.
6) Samples were further incubated at 90° C. for 10 minutes.
7) Samples were spun in microcentrifuge and were placed on ice if doing PCR immediately; otherwise they were stored at −20° C. for future use.
8) PCR quality control was run on samples using 18S RNA and 3-actin mRNA.

### Quantitative PCR on the Applied Biosystem Prism@ Instrument

Quantitative PCR was performed on the ABI Prism® 7900 Sequence Detector.

### Materials

1) 20× Primer/Probe Mix for each gene of interest.
2) 20× Primer/Probe Mix for 18s endogenous control.

### 3) 2× Taqman™ Universal PCR Master Mix.

4) cDNA transcribed from RNA extracted from cells.

### 5) Applied Biosystems 96-Well Optical Reaction Plates.

6) Applied Biosystems Optical Caps, or optical-clear film.

### 7) Applied Biosystem Prism® 7700 or 7900 Sequence Detector.

### Methods

1) Stocks of Primer/Probe mix included the Primer/Probe for the gene of interest, Primer/Probe for 18S endogenous control, and 2×PCR Master Mix were made based on the following ratio.
   For one gene with quadruplicate samples testing two conditions (2 plates), 1× (1 well) (μL): 2× Master Mix 7.5 μL
   20× 18S Primer/Probe Mix 0.75 μL
   20× Gene of interest Primer/Probe Mix 0.75 μL
   Total 9.0 μL.
   Sufficient excess was prepared to allow for pipetting error, e.g., approximately 10% excess.
2) 95 μL of cDNA was diluted into 2000 μL of water. The amount of cDNA was adjusted to give Ct values between 10 and 18, typically between 12 and 16.
3) 9 μL of Primer/Probe mix was pipetted into the appropriate wells of an Applied Biosystems 384-Well Optical Reaction Plate.
4) 10 μL of cDNA stock solution was pipetted into each well of the Applied Biosystems 384-Well Optical Reaction Plate.
5) The plate was sealed with Applied Biosystems Optical Caps, or optical-clear film.
6) The plate was then analyzed on the ABI Prism® 7900 Sequence Detector.

### Quantitative PCR Assay on Cepheid SmartCycler® Instruments

Quantitative PCR can be performed on Cepheid SmartCycler® Instruments. The experiments are typically performed in duplicate with three target genes and one reference gene in each sample.

### Materials

1) SmartMix™-HM lyophilized Master Mix.
2) Molecular grade water.
3) 20× Primer/Probe Mix for the 18S endogenous control gene. The endogenous control gene will be dual labeled with VIC-MGB or equivalent.
4) 20× Primer/Probe Mix for each for target gene 1, dual labeled with FAM-BHQ1 or equivalent.
5) 20× Primer/Probe Mix for each for target gene 2, dual labeled with Texas Red-BHQ2 or equivalent.
6) 20× Primer/Probe Mix for each for target gene 3, dual labeled with Alexa 647-BHQ3 or equivalent.
7) Tris buffer, pH 9.0.
8) cDNA reverse-transcribed from RNA extracted from sample.
9) SmartCycler® 25 μL tube.
10) Cepheid SmartCycler® instrument.

### Methods

1) For each cDNA sample to be investigated, the following are added to a sterile 650 μL tube:
   SmartMix™-HM lyophilized Master Mix 1 bead
   20× 18S Primer/Probe Mix 2.5 μL
   20× Target Gene 1 Primer/Probe Mix 2.5 μL
   20× Target Gene 2 Primer/Probe Mix 2.5 μL
   20× Target Gene 3 Primer/Probe Mix 2.5 μL
   Tris Buffer, pH 9.0 2.5 μL
   Sterile Water 34.5 μL
   Total 47 μL
   The mixture is vortexed for 1 second three times to completely mix the reagents, and then briefly centrifuged.
2) The cDNA sample is diluted so that a 3 μL addition to the reagent mixture above gives an 18S reference gene CT value between 12 and 16.
3) 3 μL of the prepared cDNA sample is added to the reagent mixture, bringing the total volume to 50 μL. The mixture is vortexed for 1 second three times to completely mix the reagents, and then briefly centrifuged.
4) 25 μL of the mixture is added to each of two SmartCycler® tubes. The tubes are spun for 5 seconds in a microcentrifuge having an adapter for SmartCycler® tubes.
5) The SmartCycler® tubes are removed from the microcentrifuge and are inspected for air bubbles. If bubbles are present, the tubes are re-spun. Otherwise, the tubes are loaded into the SmartCycler® instrument.
6) Quantitative PCR is performed on the SmartCycler® instrument. The data are exported and analyzed.
   Quantitative PCR Assay on Cepheid SmartCycler® Instruments with SmartBeads™

Quantitative PCR can be performed on Cepheid SmartCycler® Instruments. The experiments are typically performed in duplicate with three target genes and one reference gene in each sample.

### Materials

1) SmartMix™-HM lyophilized Master Mix.
2) Molecular grade water.
3) SmartBeads™ containing the 18S endogenous control gene dual labeled with VIC-MGB or equivalent, and the three target genes, one dual labeled with FAM-BHQ1 or equivalent, one dual labeled with Texas Red-BHQ2 or equivalent, and one dual labeled with Alexa 647-BHQ3 or equivalent.
4) Tris buffer, pH 9.0
5) cDNA transcribed from RNA extracted from sample.
6) SmartCycler® 25 μL tube.
7) Cepheid SmartCycler® instrument.

### Methods

1) For each cDNA sample to be investigated, the following is added to a sterile 650 μL tube:
   SmartMix™-HM lyophilized Master Mix 1 bead
   SmartBeads™ containing four primer/probe sets 1 bead
   Tris Buffer, pH 9.0 2.5 μL
   Sterile Water 44.5 μL
   Total 47 μL
   The mixture is vortexed for 1 second three times to completely mix the reagents, and then briefly centrifuged.
2) The cDNA sample is diluted so that a 3 μL addition to the reagent mixture above gave an 18S reference gene CT value between 12 and 16.
3) 3 μL of the prepared cDNA sample is added to the reagent mixture, bringing the total volume to 50 μL. The mixture is vortexed for 1 second three times to completely mix the reagents, and then briefly centrifuged.
4) 25 μL of the mixture is added to each of two SmartCycler® tubes. The tubes are spun for 5 seconds in a microcentrifuge having an adapter for SmartCycler® tubes.
5) The two SmartCycler® tubes are removed from the microcentrifuge and inspected for air bubbles. If bubbles are present, the tubes are re-spun. Otherwise, the tubes are loaded into the SmartCycler® instrument.
6) QPCR is performed on the SmartCycler® instrument. The data are exported and analyzed.

### Quantitative PCR Assay on the Cepheid GeneXpert® Instrument

Quantitative PCR can be performed on the Cepheid GeneXpert® instrument.

### Materials

1) Cepheid GeneXpert® self contained cartridge preloaded with a lyophilized SmartMix™-HM master mix bead and a lyophilized SmartBead™ containing four primer/probe sets.
2) Molecular grade water, containing Tris buffer, pH 9.0.
3) Extraction and purification reagents.
4) Clinical sample (whole blood, RNA, etc.).
5) Cepheid GeneXpert® instrument.

### Methods

1) Molecular grade water with Tris buffer, pH 9.0 is filled into appropriate chamber of GeneXpert® self-contained cartridge.
2) Extraction and purification reagents are filled into appropriate chambers of self-contained cartridge.
3) Clinical sample is loaded into appropriate chamber of self-contained cartridge.
4) The cartridge is sealed and loaded into GeneXpert® instrument.
5) The appropriate extraction and amplification protocol is performed on the GeneXpert® instrument. The result is then analyzed.

### Quantitative PCR Assay on the Roche LightCycler® 480 Real-Time PCR System

Quantitative PCR can be performed on the Roche LightCycler® 480 Real-Time PCR System.

### Materials

1) 20× Primer/Probe stock for the 18S endogenous control gene. The endogenous control gene may be dual labeled with either VIC-MGB or VIC-TAMRA.
2) 20× Primer/Probe stock for each target gene, dual labeled with either FAM-TAMRA or FAM-BHQ1.
3) 2× LightCycler® 480 Probes Master (master mix).
4) 1×cDNA sample stock reverse-transcribed from RNA extracted from each sample.
5) 1×TE buffer, pH 8.0.
6) LightCycler® 480 384-well plates.
7) Source MDx 24 gene Precision Profile™ 96-well intermediate plates.
8) RNase/DNase-free 96-well plate.
9) 1.5 mL microcentrifuge tubes.
10) Beckman/Coulter Biomek® 3000 Laboratory Automation Workstation.
11) Velocity 11 Bravo™ Liquid Handling Platform.
12) LightCycler® 480 Real-Time PCR System.

### Methods

1) A Source MDx 24 gene Precision Profile™ 96-well intermediate plate is removed from the freezer, thawed and spun in a plate centrifuge.
2) Four 1×cDNA sample stocks are diluted in separate 1.5 mL microcentrifuge tubes with the total final volume for each of 540 μL.
3) The 4 diluted cDNA samples are transferred to an empty RNase/DNase-free 96-well plate using the Biomek® 3000 Laboratory Automation Workstation.
4) The cDNA samples from the cDNA plate created in step 3 are placed in the thawed and centrifuged Source MDx 24 gene Precision Profile™ 96-well intermediate plate using Biomek® 3000 Laboratory Automation Workstation. The plate is sealed with a foil seal and spun in a plate centrifuge.
5) The contents of the cDNA-loaded Source MDx 24 gene Precision Profile™ 96-well intermediate plate are transferred to a new LightCycler® 480 384-well plate using the Bravo™ Liquid Handling Platform. The 384-well plate is sealed with a LightCycler® 480 optical sealing foil and spun in a plate centrifuge for 1 minute at 2000 rpm.
6) The sealed plate is placed in a dark 4° C. refrigerator for a minimum of 4 minutes.
7) The plate is loaded into the LightCycler® 480 Real-Time PCR System and the PCR is started with appropriate parameters.
8) At the conclusion of the run, the data are analyzed and the resulting CP values are exported to a database.

### Results

Quantitative PCR was performed on the ABI Prism® 7900 Sequence Detector system to determine the amount of RNA corresponding to specific genes in these samples.

In some instances, target gene measurements may be beyond the detection limit of the particular platform instrument used to detect and quantify constituents of a target gene. To address the issue of “undetermined” gene expression measures as lack of expression for a particular gene, the detection limit was reset and the “undetermined” constituents were “flagged.” For the ABI Prism® 7900HT Sequence Detection System, target gene FAM measurements that were beyond the detection limit of the instrument (>40 cycles) were reported as “undetermined.” Detection Limit Reset was performed when at least 1 of 3 target gene FAM CT replicates was not detected after 40 cycles.

Samples were typically run on a 384 well PCR plate in replicates of three wells for each target gene (assay). A sample was divided into aliquots. For each aliquot, the concentration of each constituent target gene was measured in a separate well of the 384 well plate. With each assay conducted in triplicate, an average coefficient of variation (in accordance with (standard deviation/average)*100) of less than 2 percent was found among the normalized ACt measurements for each assay. In this embodiment, normalized quantitation of the target mRNA was determined by the difference in threshold cycles between the internal control (e.g., an endogenous marker such as 18S rRNA, or an exogenous marker) and the gene of interest. This is a measure called “intra-assay variability.” Duplicate assays also were conducted on different occasions using the same sample material. This is a measure of “inter-assay variability.” To eliminate data points that are statistical “outliers,” data points that differed by a percentage greater than 3% from the average of three values were excluded. Moreover, if more than one data point in a set of three were excluded by this procedure, then data for the relevant constituent were discarded.

Calibrated data sets were highly reproducible in samples taken from the same individual under the same conditions. Calibrated profile data sets were also reproducible in samples that were repeatedly tested.

### Example 3: Classifying a Subject into Either (1) the Grade 3-4 Diarrhea/Colitis Group, or (2) the Grade 0-2 Diarrhea Group

Statistical analyses were performed for models and classifiers for classifying a subject into either (1) the Grade 3-4 diarrhea/colitis group, or (2) the Grade 0-2 diarrhea group.

### Model Construction

The gene set for models and classifiers for classifying a subject into either (1) the Grade 3-4 diarrhea/colitis group, or (2) the Grade 0-2 diarrhea group, includes CCR3, MMP9, and PTGS2.

One, two, three, four, or all five genes selected from the group consisting of CARD12, CCND1, IL5, F5 and GYPA can be added to the gene set that includes CCR3, MMP9, and PTGS2, to obtain more gene sets useful in the present methods. Various gene sets were built and tested (Table 3).

The levels of transcribed mRNA corresponding to the genes in each tested gene set were used as explanatory variables in logistic regression. The model was then applied to the 1008 dataset to create a classifier. Logistic regressions were first performed in the 1008 training dataset to determine the parameters for the classifier. The resulting classifier was then tested in the 1009 validation dataset. In this example, the immune-related adverse event in both the training dataset and the validation dataset was defined as diarrhea of Grade 3 or Grade 4. Thus, if a subject experienced either Grade 3 or Grade 4 diarrhea during the 12-month study period, the subject was categorized as having experienced an immune-related adverse event (“immunotherapy-intolerant”). If, throughout the 12-month study period, a subject instead experienced diarrhea that was no more severe than Grade 1 or Grade 2, or did not experience diarrhea, the subject was categorized as not having experienced an immune-related adverse event (“immunotherapy-tolerant”).

### Result

Table 3 lists several classifiers for classifying a subject into either (1) the Grade 3-4 diarrhea/colitis group, or (2) the Grade 0-2 diarrhea group, providing coefficients, logistic regression equation constant, and two AUCs for each classifier.

Classifier 9 in Table 3 was utilized in the analysis shown in Table 4. Table 4 shows the results of applying the classifier to expression data for the 150 subjects represented in the 1008 training dataset. The classifier calculated a likelihood score for each subject, and an appropriate likelihood score cut-off point was selected. A subject with a likelihood score that is higher than the cut-off point would be classified as expected to experience the immune-related adverse event. The remaining subjects were classified as not expected to experience the immune-related adverse event. As shown in Table 4, this classifier correctly classified 8 of the 9 subjects who actually experienced Grade 3 diarrhea, by classifying them as expected to experience Grade 3 or Grade 4 diarrhea. (The other 1 of the 9 was incorrectly classified as not expected to experience Grade 3 or Grade 4 diarrhea.) Of the 12 subjects who actually experienced Grade 2 diarrhea but no higher grade, the classifier correctly classified 7 as not expected to experience Grade 3 or Grade 4 diarrhea. (The other 5 of the 12 were incorrectly classified as expected to experience Grade 3 or Grade 4 diarrhea.) Of the 39 subjects who experienced Grade 1 diarrhea but no higher grade, the classifier correctly classified 33 as not expected to experience Grade 3 or Grade 4 diarrhea. (The other 6 of the 39 were incorrectly classified as expected to experience Grade 3 or Grade 4 diarrhea.) Of the 90 subjects who did not have diarrhea, 72 were correctly classified as not expected to experience Grade 3 or Grade 4 diarrhea. (The other 18 of the 90 were incorrectly classified as expected to experience Grade 3 or Grade 4 diarrhea.) Table 5 shows the sensitivity, the specificity and the negative predictive value of applying Classifier 9 in Table 3 to the 1008 training dataset.

Table 6 shows the results of applying the same classifier (Classifier 9 in Table 3) to expression data for the 210 subjects represented in the 1009 validation dataset.

Table 7 shows the sensitivity, the specificity and the negative predictive value of applying Classifier 9 in Table 3 to the 1009 validation dataset.

TABLE 3
Examples of classifiers that classify a subject into either (1) the
Grade 3-4 diarrhea/colitis group, or (2) the Grade 0-2 diarrhea group

| | | | | AUC | AUC |
|---|---|---|---|---|---|
| Classi- | | | | (training) | (Validation) |
| fier | | Coeffi- | | 1008 dataset | 1009 dataset |
| No. | Gene | cient | Constant | (N = 150) | (N = 210) |
| 1 | CCR3 | 0.392 | −25.023 | 0.8085 | 0.6778 |
| | MMP9 | −0.381 | | | |
| | PTGS2 | 1.185 | | | |
| 2 | CCR3 | 0.404 | −28.231 | 0.7890 | 0.6926 |
| | IL5 | 0.285 | | | |
| | MMP9 | −0.305 | | | |
| | PTGS2 | 0.921 | | | |
| 3 | CARD12 | −0.502 | −35.256 | 0.8597 | 0.6921 |
| | CCND1 | 0.766 | | | |
| | CCR3 | 0.39 | | | |
| | F5 | −0.516 | | | |
| | MMP9 | −0.088 | | | |
| | PTGS2 | 1.353 | | | |
| 4 | CARD12 | −0.244 | −24.982 | 0.7865 | 0.7061 |
| | CCR3 | 0.37 | | | |
| | F5 | −0.478 | | | |
| | IL5 | 0.345 | | | |
| | MMP9 | 0.003 | | | |
| | PTGS2 | 1.166 | | | |
| 5 | CARD12 | −0.842 | −36.358 | 0.8357 | 0.6914 |
| | CCND1 | 0.742 | | | |
| | CCR3 | 0.37 | | | |
| | MMP9 | −0.4 | | | |
| | PTGS2 | 1.35 | | | |
| 6 | CARD12 | −0.902 | −38.369 | 0.8463 | 0.6881 |
| | CCND1 | 0.707 | | | |
| | CCR3 | 0.367 | | | |
| | IL5 | 0.254 | | | |
| | MMP9 | 0.106 | | | |
| | PTGS2 | 1.167 | | | |
| 7 | CCND1 | 0.627 | −39.347 | 0.8507 | 0.689 |
| | CCR3 | 0.435 | | | |
| | GYPA | 0.125 | | | |
| | MMP9 | −0.364 | | | |
| | PTGS2 | 0.944 | | | |
| 8 | CCND1 | 0.627 | −39.347 | 0.8569 | 0.74 |
| | CCR3 | 0.435 | | | |
| | GYPA | 0.5 | | | |
| | MMP9 | −0.364 | | | |
| | PTGS2 | 0.944 | | | |
| 9 | CCND1 | 0.627 | −39.347 | 0.8595 | 0.7506 |
| | CCR3 | 0.435 | | | |
| | GYPA | 0.6 | | | |
| | MMP9 | −0.364 | | | |
| | PTGS2 | 0.944 | | | |
| 10 | CCND1 | 0.627 | −39.347 | 0.8424 | 0.7643 |
| | CCR3 | 0.435 | | | |
| | GYPA | 0.7 | | | |
| | MMP9 | −0.364 | | | |
| | PTGS2 | 0.944 | | | |
| 11 | CCND1 | 0.627 | −39.347 | 0.8268 | 0.7736 |
| | CCR3 | 0.435 | | | |
| | GYPA | 0.8 | | | |
| | MMP9 | −0.364 | | | |
| | PTGS2 | 0.944 | | | |

TABLE 4
Results of applying an exemplary classifier (Classifier
9 in Table 3) to expression data for the 150 subjects
represented in the 1008 training dataset

| | | | No. of | Percentage |
|---|---|---|---|---|
| | | | subjects | of subjects |
| | | Of the 150 | in that | in that |
| Grade Group | No. of | subjects, | Grade Group | Grade Group |
| (highest Grade | subjects | percentage | predicted to | predicted to |
| of diarrhea | in that | in that | experience | experience |
| actually | Grade | Grade | Grade 3 or 4 | Grade 3 or 4 |
| experienced) | Group | Group | diarrhea | diarrhea |
| Grade 4 | 0 | 0.0% | — | — |
| Grade 3 | 9 | 6.0% | 8 of 9 | 88.9% |
| Grade 2 | 12 | 8.0% | 5 of 12 | 41.7% |
| Grade 1 | 39 | 26.0% | 6 of 39 | 15.4% |
| Grade 0 | 90 | 60.0% | 18 of 90 | 20.0% |
| Total | 150 | 100.0% | 37 of 150 | 24.7% |
| (all Grades) | | | | |

TABLE 5
Statistical measures of an exemplary classifier (Classifier
9 in Table 3) in the 1008 training dataset

| Statistical measure | Results |
|---|---|
| Sensitivity | 88.9% |
| Specificity | 80.1% |
| Negative Predictive Value | 99.1% |

TABLE 6
Results of applying an exemplary classifier (Classifier
9 in Table 3) to expression data for the 210 subjects
represented in the 1009 validation dataset

| | | | No. of | Percentage |
|---|---|---|---|---|
| | | | subjects | of subjects |
| | | Of the 210 | in that | in that |
| Grade Group | No. of | subjects, | Grade Group | Grade Group |
| (highest Grade | subjects | percentage | predicted to | predicted to |
| of diarrhea | in that | in that | experience | experience |
| actually | Grade | Grade | Grade 3 or 4 | Grade 3 or 4 |
| experienced) | Group | Group | diarrhea | diarrhea |
| Grade 4 | 1 | 0.5% | 0 of 1 | 0.0% |
| Grade 3 | 26 | 12.4% | 16 of 26 | 61.5% |
| Grade 2 | 29 | 13.8% | 8 of 29 | 27.6% |
| Grade 1 | 36 | 17.1% | 5 of 36 | 13.9% |
| Grade 0 | 118 | 56.2% | 14 of 118 | 11.9% |
| Total | 210 | 100.0% | 43 of 210 | 20.5% |
| (all Grades) | | | | |

TABLE 7
Statistical measures of an exemplary classifier (Classifier
9 in Table 3) in the 1009 validation dataset

| Statistical measure | Results |
|---|---|
| Sensitivity | 59.3% |
| Specificity | 85.2% |
| Negative Predictive Value | 93.4% |

### Example 4: Classifying a Subject into Either (1) the Grade 2-4 Diarrhea/Colitis Group, or (2) the Grade 0-1 Diarrhea Group

Statistical analyses were performed for models and classifiers for classifying a subject into either (1) the Grade 2-4 diarrhea/colitis group, or (2) the Grade 0-1 diarrhea group.

### Model Construction

The gene set for models and classifiers for classifying a subject into either (1) the Grade 2-4 diarrhea/colitis group, or (2) the Grade 0-1 diarrhea group includes CCL3, CCR3, IL8, and PTGS2.

One, two, three, four, five, six, seven, eight, nine, ten, eleven, twelve, thirteen, fourteen, fifteen, or all sixteen genes selected from the group consisting of CARD12, CDC25A, CXCL1, F5, FAM210, GADD45A, IL18BP, IL2RA, IL5, IRAK3, ITGA4, MAPK14, MMP9, SOCS3, TLR9, and UBE2C can be added to the gene set that includes CCL3, CCR3, IL8, and PTGS2 to obtain a new gene set. Various gene sets were built and tested (Table 8). Among these gene sets, some of them have one, two, three, four, five, or all six genes selected from the group consisting of CARD12, F5, MMP9, SOCS3, IL5 and TLR9, as well as all of CCL3, CCR3, IL8, and PTGS2 (e.g., Classifiers 2-5 in Table 8). In some embodiments, the gene set includes not only CCL3, CCR3, IL8, and PTGS2, but also CARD12, CXCL1, F5, FAM210, GADD45A, IL18BP, IL2RA, IL5, IRAK3, ITGA4, MAPK14, MMP9, SOCS3, TLR9, and UBE2C (e.g., Classifier 16 in Table 8).

The levels of transcribed mRNA corresponding to the genes in each tested gene set were used as explanatory variables in logistic regression. The model was then applied to the 1008 training dataset to create a classifier. Logistic regressions were first performed in the 1008 training dataset to determine the parameters for the classifier. The resulting classifier was then tested in the 1009 validation dataset. In this example (unlike Example 3), the immune-related adverse event in both the training dataset and the validation dataset was defined as diarrhea of any of Grade 2, Grade 3, or Grade 4. Thus, if a subject experienced Grade 2, Grade 3, or Grade 4 diarrhea during the 12-month study period, the subject was categorized as having experienced an immune-related adverse event (“immunotherapy-intolerant”). If, throughout the 12-month study period, a subject instead experienced diarrhea that was no more severe than Grade 1, or did not experience diarrhea (“Grade 0 diarrhea”), the subject was categorized as not having experienced an immune-related adverse event (“immunotherapy-tolerant”).

### Result

Table 8 lists several classifiers for classifying a subject into either (1) the Grade 2-4 diarrhea/colitis group, or (2) the Grade 0-1 diarrhea group, providing coefficients, logistic regression equation constant, and two AUCs for each classifier.

Classifier 4 in Table 8 was utilized in the analysis shown in Table 9. Table 9 shows the results of applying the classifier to expression data for the 150 subjects represented in the 1008 training dataset. The classifier calculated a likelihood score for each subject, and an appropriate likelihood score cut-off point was selected. A subject with a likelihood score that is higher than the cut-off point would be classified as expected to experience the immune-related adverse event. The remaining subjects were classified as not expected to experience the immune-related adverse event. As shown in Table 9, Classifier 4 correctly classified 7 of the 9 subjects who actually experienced Grade 3 diarrhea, by classifying them as expected to experience Grade 2, Grade 3, or Grade 4 diarrhea. (The other 2 of the 9 were incorrectly classified as not expected to experience Grade 2, Grade 3, or Grade 4 diarrhea.) The classifier correctly classified 6 of the 12 subjects who experienced Grade 2 diarrhea but no higher grade, classifying them as expected to experience Grade 2, Grade 3 or Grade 4 diarrhea. (The other 6 of the 12 were incorrectly classified as not expected to experience Grade 2, Grade 3, or Grade 4 diarrhea.) Of the 39 subjects who experienced Grade 1 diarrhea but no higher grade, 35 were correctly classified as not expected to experience Grade 2, Grade 3, or Grade 4 diarrhea. (The other 4 of the 39 were incorrectly classified as expected to experience Grade 2, Grade 3, or Grade 4 diarrhea.) Among the 90 subjects who did not experience diarrhea, 79 were correctly classified as not expected to experience Grade 2, Grade 3, or Grade 4 diarrhea. (The other 11 of the 90 were incorrectly classified as expected to experience Grade 2, Grade 3, or Grade 4 diarrhea.)

Table 10 shows the sensitivity, the specificity and the negative predictive value of applying Classifier 4 in Table 8 to the 1008 training dataset.

Table 11 shows the results of applying the same classifier (Classifier 4 in Table 8) to expression data for the 210 subjects represented in the 1009 validation dataset.

Table 12 shows the sensitivity, the specificity and the negative predictive value of applying Classifier 4 in Table 8 to the 1009 validation dataset.

Classifier 15 of Table 8 was also utilized in the analysis shown in Table 13. Table 13 shows the results of applying the classifier to expression data for the 150 subjects represented in the 1008 training dataset.

Table 14 shows the sensitivity, the specificity, the negative predictive value, the positive predictive value, and the AUC of applying Classifier 15 of Table 8 to the 1008 training dataset.

Table 15 shows the results of applying the same classifier (Classifier 15 of Table 8) to expression data for the 210 subjects represented in the 1009 validation dataset.

Table 16 shows the sensitivity, the specificity, the negative predictive value, the positive predictive value, and the AUC of applying Classifier 15 of Table 8 to the 1009 validation dataset.

When Classifier 15 in Table 8 was applied to the 1008 training dataset and the 1009 validation set, the cut-off point for the likelihood score was selected to maximize the positive predictive value (the proportions of true positives in the group of both true positives and false positives). This cut-off point happened to be the same, −0.29, for both the 1008 training dataset and the 1009 validation dataset. Thus, the sensitivities, the specificities, the negative predictive values, and the positive predictive values in Tables 14 and 16 were calculated using the same cut-off point. As the subjects in 1008 training population received chemotherapy before being treated with the immunotherapy while those in the 1009 validation population did not, the fact that both datasets had the same cut-off point at least suggests that the biomarkers and the classifiers hold true among subjects that received chemotherapy and subjects that did not receive chemotherapy prior to being treated with an immunotherapy.

In further work, the cut-off point was adjusted to increase negative predictive value (the proportion of true negatives in the group of both true negatives and false negatives). With a low cut-off point of −2.4, Classifier 15 in Table 8 categorized 84 (56%) out of 150 subjects in the 1008 training population as not expected to experience Grade 2, Grade 3, or Grade 4 diarrhea. Among the 84 subjects, only two actually experienced Grade 2 diarrhea, one actually experienced Grade 3 diarrhea, and none actually experienced Grade 4 diarrhea (no patient in the entire 1008 training population experienced Grade 4 diarrhea). Using the same cut off point of −2.4, Classifier 15 in Table 8 categorized 81 (39%) out of 210 subjects in the 1009 validation population as not expected to experience Grade 2, Grade 3, or Grade 4 diarrhea. Of these 81 subjects, only four subjects actually experienced Grade 2 diarrhea, one subject actually experienced Grade 4 diarrhea, and none experienced Grade 3 diarrhea.

Table 17 shows the results of applying Classifier 16 of Table 8 to expression data for the 150 subjects represented in the 1008 training dataset.

Table 18 shows the sensitivity, the specificity, the negative predictive value, the positive predictive value, and the AUC of applying Classifier 16 of Table 8 to the 1008 training dataset.

Table 19 shows the results of applying Classifier 16 of Table 8 to expression data for the 210 subjects represented in the 1009 validation dataset.

Table 20 shows the sensitivity, the specificity, the negative predictive value, the positive predictive value, and the AUC of applying Classifier 16 of Table 8 to the 1009 validation dataset.

When Classifier 16 in Table 8 was applied to the 1008 training dataset and the 1009 validation set, the cut-off point was set to zero for both the 1008 training dataset and the 1009 validation dataset. Thus, the sensitivities, the specificities, the negative predictive values, and the positive predictive values in Tables 17 and 19 were calculated using the same cut-off point. In a further analysis, the cut-off point was adjusted to increase negative predictive value. With a low cut-off point of −2.0, Classifier 16 in Table 8 categorized 91 (61%) out of 150 subjects in the 1008 training population as not expected to experience Grade 2, Grade 3, or Grade 4 diarrhea. Among those 91 subjects, only three actually experienced Grade 2 diarrhea, one actually experienced Grade 3 diarrhea, and none experienced Grade 4 diarrhea (no patient in the entire 1008 patient population experienced Grade 4 diarrhea). Using the same cut-off point of −2.0, Classifier 16 in Table 8 categorized 90 (43%) out of 210 subjects in the 1009 validation population as not expected to experience Grade 2, Grade 3, or Grade 4 diarrhea. Of these 90 subjects, only five subjects actually experienced Grade 2 diarrhea, two subjects actually experienced Grade 3 diarrhea, and none experienced Grade 4 diarrhea.

TABLE 8
Examples of classifiers that classify a subject into either (1) the
Grade 2-4 diarrhea/colitis group, or (2) the Grade 0-1 diarrhea group

| | | | | AUC | AUC |
|---|---|---|---|---|---|
| Classi- | | | | (training) | (Validation) |
| fier | | Coeffi- | | 1008 dataset | 1009 dataset |
| No. | Gene | cient | Constant | (N = 150) | (N = 210) |
| 1 | CCL3 | 0.672 | −32.05 | 0.7434 | 0.6623 |
| | CCR3 | 0.509 | | | |
| | IL8 | 0.342 | | | |
| | PTGS2 | 0.3 | | | |
| 2 | CCL3 | 0.769 | −28.326 | 0.7844 | 0.6993 |
| | CCR3 | 0.389 | | | |
| | F5 | −0.952 | | | |
| | IL5 | 0.228 | | | |
| | IL8 | −0.2 | | | |
| | PTGS2 | 0.769 | | | |
| 3 | CARD12 | −0.602 | −28.018 | 0.7896 | 0.7039 |
| | CCL3 | 0.822 | | | |
| | CCR3 | 0.358 | | | |
| | F5 | −0.53 | | | |
| | IL5 | 0.222 | | | |
| | IL8 | 0.074 | | | |
| | PTGS2 | 0.92 | | | |
| 4 | CARD12 | −0.509 | −26.585 | 0.7914 | 0.7035 |
| | CCL3 | 0.86 | | | |
| | CCR3 | 0.406 | | | |
| | F5 | −0.494 | | | |
| | IL5 | 0.224 | | | |
| | IL8 | 0.121 | | | |
| | PTGS2 | 0.944 | | | |
| | TLR9 | −0.377 | | | |
| 5 | CARD12 | −0.759 | −28.064 | 0.7933 | 0.7025 |
| | CCL3 | 0.82 | | | |
| | CCR3 | 0.424 | | | |
| | F5 | −0.644 | | | |
| | IL5 | 0.262 | | | |
| | IL8 | 0.159 | | | |
| | MMP9 | 0.266 | | | |
| | PTGS2 | 0.79 | | | |
| | SOCS3 | −0.033 | | | |
| 6 | CCL3 | 0.755 | −20.483 | 0.7573 | 0.6828 |
| | CCR3 | 0.488 | | | |
| | GADD45A | −0.724 | | | |
| | IL8 | 0.134 | | | |
| | PTGS2 | 0.338 | | | |
| 7 | CCL3 | 0.879 | −24.995 | 0.7649 | 0.6977 |
| | CCR3 | 0.403 | | | |
| | GADD45A | −0.359 | | | |
| | IL8 | 0.175 | | | |
| | PTGS2 | 0.502 | | | |
| | SOCS3 | −0.421 | | | |
| 8 | CARD12 | −0.735 | −26.903 | 0.7936 | 0.702 |
| | CCL3 | 0.812 | | | |
| | CCR3 | 0.434 | | | |
| | F5 | −0.651 | | | |
| | GADD45A | −0.09 | | | |
| | IL5 | 0.263 | | | |
| | IL8 | 0.148 | | | |
| | MMP9 | 0.274 | | | |
| | PTGS2 | 0.782 | | | |
| 9 | CARD12 | −0.6 | −22.693 | 0.8172 | 0.7075 |
| | CCL3 | 1.218 | | | |
| | CCR3 | 0.745 | | | |
| | F5 | −0.139 | | | |
| | IL18BP | −1.176 | | | |
| | IL2RA | −0.386 | | | |
| | IL5 | 0.396 | | | |
| | IL8 | 0.212 | | | |
| | ITGA4 | 0.114 | | | |
| | MMP9 | −0.056 | | | |
| | PTGS2 | 0.506 | | | |
| 10 | CARD12 | −1.303 | −21.353 | 0.8299 | 0.7135 |
| | CCL3 | 1.213 | | | |
| | CCR3 | 0.795 | | | |
| | F5 | −0.219 | | | |
| | IL18BP | −1.277 | | | |
| | IL2RA | −0.362 | | | |
| | IL5 | 0.404 | | | |
| | IL8 | 0.235 | | | |
| | IRAK3 | 0.698 | | | |
| | ITGA4 | 0.015 | | | |
| | MAPK14 | 0.366 | | | |
| | MMP9 | −0.184 | | | |
| | PTGS2 | 0.418 | | | |
| 11 | CARD12 | −1.418 | −19.921 | 0.8281 | 0.7286 |
| | CCL3 | 1.27 | | | |
| | CCR3 | 0.828 | | | |
| | CXCL1 | −0.949 | | | |
| | F5 | −0.243 | | | |
| | IL18BP | −1.623 | | | |
| | IL2RA | −0.448 | | | |
| | IL5 | 0.455 | | | |
| | IL8 | 0.415 | | | |
| | IRAK3 | 0.753 | | | |
| | ITGA4 | −0.133 | | | |
| | MAPK14 | 0.234 | | | |
| | MMP9 | 0.06 | | | |
| | PTGS2 | 0.625 | | | |
| | TLR9 | 0.929 | | | |
| 12 | FAM210 | 0.815 | −45.269 | 0.8475 | 0.7592 |
| | CARD12 | −0.865 | | | |
| | CCL3 | 1.189 | | | |
| | CCR3 | 0.8 | | | |
| | CXCL1 | −1.01 | | | |
| | F5 | −0.568 | | | |
| | IL18BP | −1.806 | | | |
| | IL2RA | 0.083 | | | |
| | IL5 | 0.387 | | | |
| | IL8 | 0.655 | | | |
| | IRAK3 | 0.423 | | | |
| | ITGA4 | 0.098 | | | |
| | MAPK14 | 0.092 | | | |
| | MMP9 | 0.084 | | | |
| | PTGS2 | 0.769 | | | |
| | TLR9 | 1.092 | | | |
| 13 | FAM210 | 0.846 | −40.881 | 0.8537 | 0.7785 |
| | CARD12 | −0.748 | | | |
| | CCL3 | 1.166 | | | |
| | CCR3 | 0.867 | | | |
| | CXCL1 | −1.122 | | | |
| | F5 | −0.465 | | | |
| | IL18BP | −1.69 | | | |
| | IL2RA | 0.034 | | | |
| | IL5 | 0.441 | | | |
| | IL8 | 0.64 | | | |
| | IRAK3 | 0.516 | | | |
| | ITGA4 | 0.064 | | | |
| | MAPK14 | 0.153 | | | |
| | MMP9 | −0.03 | | | |
| | PTGS2 | 0.826 | | | |
| | TLR9 | 1.328 | | | |
| | UBE2C | −0.726 | | | |
| 14 | FAM210 | 1.128 | −30.855 | 0.8674 | 0.7849 |
| | CARD12 | −0.517 | | | |
| | CCL3 | 1.238 | | | |
| | CCR3 | 1.213 | | | |
| | CXCL1 | −1.163 | | | |
| | F5 | −0.573 | | | |
| | GADD45A | −1.401 | | | |
| | IL18BP | −1.892 | | | |
| | IL2RA | 0.214 | | | |
| | IL5 | 0.423 | | | |
| | IL8 | 0.545 | | | |
| | IRAK3 | 0.986 | | | |
| | ITGA4 | −0.074 | | | |
| | MAPK14 | 0.182 | | | |
| | MMP9 | 0.005 | | | |
| | PTGS2 | 0.637 | | | |
| | SOCS3 | 0.236 | | | |
| | TLR9 | 1.386 | | | |
| | UBE2C | −0.797 | | | |
| 15 | FAM210 | 1.281 | −36.696 | 0.8754 | 0.7792 |
| | CARD12 | −0.502 | | | |
| | CCL3 | 1.165 | | | |
| | CCR3 | 1.304 | | | |
| | CDC25A | 0.782 | | | |
| | CXCL1 | −1.192 | | | |
| | F5 | −0.46 | | | |
| | GADD45A | −1.349 | | | |
| | IL18BP | −2.085 | | | |
| | IL2RA | 0.184 | | | |
| | IL5 | 0.438 | | | |
| | IL8 | 0.685 | | | |
| | IRAK3 | 0.904 | | | |
| | ITGA4 | −0.223 | | | |
| | MAPK14 | −0.181 | | | |
| | MMP9 | 0.042 | | | |
| | PTGS2 | 0.703 | | | |
| | SOCS3 | 0.272 | | | |
| | TLR9 | 1.713 | | | |
| | UBE2C | −1.55 | | | |
| 16 | FAM210 | 0.732 | −23.94 | 0.8359 | 0.7902 |
| | CARD12 | −0.946 | | | |
| | CCL3 | 0.851 | | | |
| | CCR3 | 0.806 | | | |
| | CXCL1 | −0.83 | | | |
| | F5 | −0.446 | | | |
| | GADD45A | −0.008 | | | |
| | IL18BP | −0.554 | | | |
| | IL2RA | −0.219 | | | |
| | IL5 | 0.354 | | | |
| | IL8 | 0.656 | | | |
| | IRAK3 | 1.501 | | | |
| | ITGA4 | −0.17 | | | |
| | MAPK14 | −0.239 | | | |
| | MMP9 | −0.15 | | | |
| | PTGS2 | 0.38 | | | |
| | SOCS3 | −0.281 | | | |
| | TLR9 | 0.98 | | | |
| | UBE2C | −1.194 | | | |

TABLE 9
Results of applying an exemplary classifier (Classifier
4 in Table 8) to expression data for the 150 subjects
represented in the 1008 training dataset

| | | | No. of | Percentage |
|---|---|---|---|---|
| | | | subjects | of subjects |
| | | Of the 150 | in that | in that |
| Grade Group | No. of | subjects, | Grade Group | Grade Group |
| (highest Grade | subjects | percentage | predicted to | predicted to |
| of diarrhea | in that | in that | experience | experience |
| actually | Grade | Grade | Grade 2, 3 | Grade 2, 3 or |
| experienced) | Group | Group | or 4 diarrhea | 4 diarrhea |
| Grade 4 | 0 | 0.0% | — | — |
| Grade 3 | 9 | 6.0% | 7 of 9 | 77.8% |
| Grade 2 | 12 | 8.0% | 6 of 12 | 50.0% |
| Grade 1 | 39 | 26.0% | 4 of 39 | 10.3% |
| Grade 0 | 90 | 60.0% | 11 of 90 | 12.2% |
| Total | 150 | 100.0% | 28 of 150 | 18.7% |
| (all Grades) | | | | |

TABLE 10
Statistical measures of an exemplary classifier (Classifier
4 in Table 8) in the 1008 training dataset

| Statistical measure | Results |
|---|---|
| Sensitivity | 61.9% |
| Specificity | 88.4% |
| Negative Predictive Value | 93.4% |

TABLE 11
Results of applying an exemplary classifier (Classifier 4 in Table 8) to expression
data for the 210 subjects represented in the 1009 validation dataset

| | | | No. of subjects | Percentage of |
|---|---|---|---|---|
| Grade Group | | Of the 210 | in that Grade | subjects in that |
| (highest Grade | No. of | subjects, | Group predicted | Grade Group |
| of diarrhea | subjects in | percentage in | to experience | predicted to |
| actually | that Grade | that Grade | Grade 2, 3 or 4 | experience Grade |
| experienced) | Group | Group | diarrhea | 2, 3 or 4 diarrhea |
| Grade 4 | 1 | 0.5% | 0 of 1 | 0.0% |
| Grade 3 | 26 | 12.4% | 17 of 26 | 65.4% |
| Grade 2 | 29 | 13.8% | 15 of 29 | 51.7% |
| Grade 1 | 36 | 17.1% | 9 of 36 | 25.0% |
| Grade 0 | 118 | 56.2% | 23 of 118 | 19.5% |
| Total (all Grades) | 210 | 100.0% | 64 of 210 | 30.5% |

TABLE 12
Statistical measures of an exemplary classifier (Classifier
4 in Table 8) in the 1009 validation dataset

| Statistical measure | Results |
|---|---|
| Sensitivity | 57.1% |
| Specificity | 79.2% |
| Negative Predictive Value | 83.6% |

TABLE 13
Results of applying an exemplary classifier (Classifier 15 in Table 8) to expression
data for the 150 subjects represented in the 1008 training dataset

| | | | | Percentage of |
|---|---|---|---|---|
| | | | No. of subjects | subjects in that |
| Grade Group | No. of | Of the 150 | in that Grade | Grade Group |
| (highest Grade | subjects | subjects, | Group predicted | predicted to |
| of diarrhea | in that | percentage in | to experience | experience Grade |
| actually | Grade | that Grade | Grade 2, 3 or 4 | 2, 3 or 4 |
| experienced) | Group | Group | diarrhea | diarrhea |
| Grade 4 | 0 | 0.0% | — | — |
| Grade 3 | 9 | 6.0% | 6 of 9 | 66.7% |
| Grade 2 | 12 | 8.0% | 5 of 12 | 41.7% |
| Grade 1 | 39 | 26.0% | 0 of 39 | 0.0% |
| Grade 0 | 90 | 60.0% | 1 of 90 | 1.1% |
| Total (all Grades) | 150 | 100.0% | 12 of 150 | 8.0% |

TABLE 14
Statistical measures of an exemplary classifier (Classifier
15 in Table 8) in the 1008 training dataset

| Statistical measure | Results |
|---|---|
| Sensitivity | 52.4% |
| Specificity | 99.2% |
| Negative Predictive Value | 92.8% |
| Positive Predictive Value | 91.7% |
| AUC | 0.8754 |

TABLE 15
Results of applying an exemplary classifier (Classifier 15 in Table 8) to expression
data for the 210 subjects represented in the 1009 validation dataset

| | | | | Percentage of |
|---|---|---|---|---|
| | | | No. of subjects | subjects in that |
| Grade Group | | Of the 210 | in that Grade | Grade Group |
| (highest Grade | No. of | subjects, | Group predicted | predicted to |
| of diarrhea | subjects | percentage | to experience | experience Grade |
| actually | in that | in that | Grade 2, 3 or 4 | 2, 3 or 4 |
| experienced) | Grade Group | Grade Group | diarrhea | diarrhea |
| Grade 4 | 1 | 0.5% | 0 of 1 | 0.0% |
| Grade 3 | 26 | 12.4% | 18 of 26 | 69.2% |
| Grade 2 | 29 | 13.8% | 17 of 29 | 58.6% |
| Grade 1 | 36 | 17.1% | 8 of 36 | 22.2% |
| Grade 0 | 118 | 56.2% | 19 of 118 | 16.1% |
| Total (all Grades) | 210 | 100.0% | 62 of 210 | 29.5% |

TABLE 16
Statistical measures of an exemplary classifier (Classifier
15 in Table 8) in the 1009 validation dataset

| Statistical measure | Results |
|---|---|
| Sensitivity | 62.5% |
| Specificity | 82.5% |
| Negative Predictive Value | 85.8% |
| Positive Predictive Value | 56.5% |
| AUC | 0.7792 |

TABLE 17
Results of applying an exemplary classifier (Classifier 16 in Table 8) to expression
data for the 150 subjects represented in the 1008 training dataset

| | | | | Percentage of |
|---|---|---|---|---|
| | | | No. of subjects | subjects in |
| Grade Group | No. of | Of the 150 | in that Grade | that Grade Group |
| (highest Grade | subjects | subjects, | Group predicted | predicted to |
| of diarrhea | in that | percentage in | to experience | experience Grade |
| actually | Grade | that Grade | Grade 2, 3 or 4 | 2, 3 or 4 |
| experienced) | Group | Group | diarrhea | diarrhea |
| Grade 4 | 0 | 0.0% | — | — |
| Grade 3 | 9 | 6.0% | 6 of 9 | 66.7% |
| Grade 2 | 12 | 8.0% | 6 of 12 | 50.0% |
| Grade 1 | 39 | 26.0% | 1 of 39 | 2.6% |
| Grade 0 | 90 | 60.0% | 0 of 90 | 0.0% |
| Total (all Grades) | 150 | 100.0% | 13 of 150 | 8.7% |

TABLE 18
Statistical measures of an exemplary classifier (Classifier
16 in Table 8) in the 1008 training dataset

| Statistical measure | Results |
|---|---|
| Sensitivity | 57.1% |
| Specificity | 99.2% |
| Negative Predictive Value | 93.4% |
| Positive Predictive Value | 92.3% |
| AUC | 0.8359 |

TABLE 19
Results of applying an exemplary classifier (Classifier 16 in Table 8) to expression
data for the 210 subjects represented in the 1009 validation dataset

| | | | | Percentage of |
|---|---|---|---|---|
| | | | No. of subjects | subjects in that |
| Grade Group | | Of the 210 | in that Grade | Grade Group |
| (highest Grade | No. of | subjects, | Group predicted | predicted to |
| of diarrhea | subjects in | percentage | to experience | experience Grade |
| actually | that Grade | in that | Grade 2, 3 or 4 | 2, 3 or 4 |
| experienced) | Group | Grade Group | diarrhea | diarrhea |
| Grade 4 | 1 | 0.5% | 0 of 1 | 0.0% |
| Grade 3 | 26 | 12.4% | 16 of 26 | 61.5% |
| Grade 2 | 29 | 13.8% | 17 of 29 | 58.6% |
| Grade 1 | 36 | 17.1% | 1 of 36 | 2.8% |
| Grade 0 | 118 | 56.2% | 13 of 118 | 11.0% |
| Total (all Grades) | 210 | 100.0% | 47 of 210 | 22.4% |

TABLE 20
Statistical measures of an exemplary classifier (Classifier
16 in Table 8) in the 1009 validation dataset

| Statistical measure | Results |
|---|---|
| Sensitivity | 58.9% |
| Specificity | 90.9% |
| Negative Predictive Value | 85.9% |
| Positive Predictive Value | 70.2% |
| AUC | 0.7902 |

### Example 5: Classifying a Subject Who has Mild Diarrhea into Either (1) the Grade 2-4 Diarrhea/Colitis Group, or (2) the Grade 1 Diarrhea Group

Statistical analyses were performed for models and classifiers for classifying a subject who has presented with early symptoms of mild (Grade 1) diarrhea into either (1) the Grade 2-4 diarrhea/colitis group, or (2) the Grade 1 diarrhea group.

### Model Construction

Any of the gene sets, models, and classifiers described above for classifying a subject into either (1) the Grade 2-4 diarrhea/colitis group, or (2) the Grade 0-1 diarrhea group can also be used to classify a subject who has Grade 1 diarrhea into either (1) the Grade 2-4 diarrhea/colitis group, or (2) the Grade 1 diarrhea group, i.e., to predict whether the subject is likely to progress to Grade 2-4 diarrhea/colitis (i.e., is classified as “immunotherapy intolerant” for purposes of this method), or instead is likely to experience no diarrhea more severe than Grade 1 (i.e., is classified as “immunotherapy tolerant” for purposes of this method).

The gene set used in this case includes CCL3, CCR3, IL8, and PTGS2. One, two, three, four, five, six, seven, eight, nine, ten, eleven, twelve, thirteen, fourteen, fifteen, or all sixteen genes selected from the group consisting of CARD12, CDC25A, CXCL1, F5, FAM210, GADD45A, IL18BP, IL2RA, IL5, IRAK3, ITGA4, MAPK14, MMP9, SOCS3, TLR9, and UBE2C can be added to the gene set that includes CCL3, CCR3, IL8, and PTGS2, to obtain a new gene set. In some embodiments, the gene set includes not only CCL3, CCR3, IL8, and PTGS2, but also CARD12, CXCL1, F5, FAM210, GADD45A, IL18BP, IL2RA, IL5, IRAK3, ITGA4, MAPK14, MMP9, SOCS3, TLR9, and UBE2C (e.g., Classifier 1 in Table 21).

To identify useful gene sets and classifiers, the levels of transcribed mRNA corresponding to the genes in each tested gene set were used as explanatory variables in logistic regression. The model was then applied to a limited training dataset that was derived from the 1008 training dataset but includes data solely from the 60 subjects who actually experienced some level of diarrhea (Grade 1-4) during the period of the 1008 clinical trial, to create a classifier. Logistic regressions were first performed in this limited training dataset to determine the parameters for the classifier. The resulting classifier was then tested in a limited validation dataset that was derived from the 1009 validation dataset but includes data solely from the 92 subjects who actually experienced some level of diarrhea (Grade 1-4) during the period of the 1009 clinical trial. Thus, in this example (unlike Example 4), all data used for training and validation were from subjects who ultimately experienced some level of diarrhea during the treatment period. The results, described below, expand on the Example 4 results to show that the models and classifiers are able not only to distinguish the group predicted to experience Grade 2-4 diarrhea/colitis from the group predicted instead to experience no diarrhea or Grade 1 diarrhea (i.e., as in Example 4), but also are able to distinguish the group predicted to experience Grade 2-4 diarrhea/colitis from the group predicted instead to experience Grade 1 diarrhea (omitting “no diarrhea” from the latter group). This is important because it means that the models and classifiers can be applied to a blood sample taken from an immunotherapy patient who has already begun to show symptoms of mild diarrhea (Grade 1), and the results used to predict whether that patient is likely to progress to Grade 2-4 diarrhea and thus should be immediately started on aggressive prophylactic anti-inflammatory therapy, or is unlikely to progress and so need not begin that prophylactic therapy.

### Result

Table 21 lists an exemplary classifier for classifying a subject into either (1) the Grade 2-4 diarrhea/colitis group, or (2) the Grade 1 diarrhea group, providing coefficients, logistic regression equation constant, and two AUCs for each classifier. The gene set for this classifier is the same as the gene set in Classifier 16 of Table 8.

Table 22 shows the results of applying the classifier to expression data for the 60 subjects represented in the limited training dataset, with the likelihood score cut-off point being set to 0 (a score greater than 0 means that the subject is likely to develop Grade 2-4 diarrhea).

Table 23 shows the sensitivity, the specificity, the negative predictive value, the positive predictive value, and the AUC of applying Classifier 1 in Table 21 to the limited training dataset.

Table 24 shows the results of applying Classifier 1 in Table 21 to expression data for the 92 subjects represented in the limited validation dataset, with the likelihood score cut-off point similarly being set to 0.

Table 25 shows the sensitivity, the specificity, the negative predictive value, the positive predictive value, and the AUC of applying Classifier 1 in Table 21 to the limited validation dataset.

TABLE 21
An exemplary classifier that classifies a subject into either (1) the
Grade 2-4 diarrhea/colitis group, or (2) the Grade 1 diarrhea group

| | | | | AUC | AUC |
|---|---|---|---|---|---|
| | | | | (training | (Validation |
| Classifier | | | | dataset) | dataset) |
| No. | Gene | Coefficient | Constant | (N = 60) | (N = 92) |
| 1 | FAM210 | 0.732 | −25.61 | 0.827 | 0.787 |
| | CARD12 | −0.946 | | | |
| | CCL3 | 0.851 | | | |
| | CCR3 | 0.806 | | | |
| | CXCL1 | −0.83 | | | |
| | F5 | −0.446 | | | |
| | GADD45A | −0.008 | | | |
| | IL18BP | −0.554 | | | |
| | IL2RA | −0.219 | | | |
| | IL5 | 0.354 | | | |
| | IL8 | 0.656 | | | |
| | IRAK3 | 1.501 | | | |
| | ITGA4 | −0.17 | | | |
| | MAPK14 | −0.239 | | | |
| | MMP9 | −0.15 | | | |
| | PTGS2 | 0.38 | | | |
| | SOCS3 | −0.281 | | | |
| | TLR9 | 0.98 | | | |
| | UBE2C | −1.194 | | | |

TABLE 22
Results of applying an exemplary classifier (Classifier 1 in Table 21) to expression
data for the 60 subjects represented in the limited training dataset

| | | | | Percentage of |
|---|---|---|---|---|
| | | | No. of subjects | subjects in |
| Grade Group | No. of | Of the 60 | in that Grade | that Grade |
| (highest Grade | subjects | subjects, | Group predicted | Group predicted |
| of diarrhea | in that | percentage in | to experience | to experience |
| actually | Grade | that Grade | Grade 2, 3 or 4 | Grade 2, 3 or |
| experienced) | Group | Group | diarrhea | 4 diarrhea |
| Grade 4 | 0 | 0.0% | — | — |
| Grade 3 | 9 | 15.0% | 6 of 9 | 66.7% |
| Grade 2 | 12 | 20.0% | 6 of 12 | 50.0% |
| Grade 1 | 39 | 65.0% | 1 of 39 | 2.6% |
| Total (all Grades) | 60 | 100.0% | 13 of 60 | 21.7% |

TABLE 23
Statistical measures of an exemplary classifier (Classifier
1 in Table 21) in the limited training dataset

| Statistical measure | Results |
|---|---|
| Sensitivity | 57.1% |
| Specificity | 97.4% |
| Negative Predictive Value | 80.9% |
| Positive Predictive Value | 92.3% |
| AUC | 0.827 |

TABLE 24
Results of applying an exemplary classifier (Classifier 1 in Table 21) to expression
data for the 92 subjects represented in the limited validation dataset

| | | | No. of subjects | Percentage of |
|---|---|---|---|---|
| Grade Group | No. of | Of the 92 | in that Grade | subjects in that |
| (highest Grade | subjects | subjects, | Group predicted | Grade Group |
| of diarrhea | in that | percentage in | to experience | predicted to |
| actually | Grade | that Grade | Grade 2, 3 or 4 | experience Grade |
| experienced) | Group | Group | diarrhea | 2, 3 or 4 diarrhea |
| Grade 4 | 1 | 1.1% | 0 of 1 | 0.0% |
| Grade 3 | 26 | 28.3% | 16 of 26 | 61.5% |
| Grade 2 | 29 | 31.5% | 17 of 29 | 58.6% |
| Grade 1 | 36 | 39.1% | 2 of 36 | 5.6% |
| Total (all Grades) | 92 | 100.0% | 35 of 92 | 38.0% |

TABLE 25
Statistical measures of an exemplary classifier (Classifier
1 in Table 21) in the limited validation dataset

| Statistical measure | Results |
|---|---|
| Sensitivity | 58.9% |
| Specificity | 94.4% |
| Negative Predictive Value | 59.6% |
| Positive Predictive Value | 94.3% |
| AUC | 0.787 |

### OTHER EMBODIMENTS

It is to be understood that, while the disclosure has been described in conjunction with the detailed description thereof, the foregoing description is intended to illustrate and not limit the scope of the disclosure.

For example, implementations of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Implementations of the subject matter described in this specification can be implemented as one or more computer programs, i.e., one or more modules of computer program instructions encoded on a tangible program carrier for execution by, or to control the operation of, a processing device. Alternatively, or in addition, the program instructions can be encoded on a propagated signal that is an artificially generated signal, e.g., a machine-generated electrical, optical, or electromagnetic signal that is generated to encode information for transmission to suitable receiver apparatus for execution by a processing device. A machine-readable medium can be a machine-readable storage device, a machine-readable storage substrate, a random or serial access memory device, or a combination of one or more of them.

In some embodiments, various methods and formulae are implemented, in the form of computer program instructions, and executed by a processing device. Suitable programming languages for expressing the program instructions include, but are not limited to, C, C++, an embodiment of FORTRAN such as FORTRAN77 or FORTRAN90, Java, Visual Basic, Perl, Tcl/Tk, JavaScript, ADA, and statistical analysis software, such as SAS, R, MATLAB, SPSS, and Stata etc. Various aspects of the methods may be written in different computing languages from one another, and the various aspects are caused to communicate with one another by appropriate system-level-tools available on a given system.

The processes and logic flows described in this specification can be performed by one or more programmable computers executing one or more computer programs to perform functions by operating on input information and generating output. The processes and logic flows can also be performed by, and apparatus can also be implemented as, special purpose logic circuitry, e.g., an FPGA (field programmable gate array) or an ASIC (application specific integrated circuit) or RISC.

Computers suitable for the execution of a computer program include, by way of example, general or special purpose microprocessors or both, or any other kind of central processing unit. Generally, a central processing unit will receive instructions and information from a read only memory or a random access memory or both. The essential elements of a computer are a central processing unit for performing or executing instructions and one or more memory devices for storing instructions and information. Generally, a computer will also include, or be operatively coupled to receive information from or transfer information to, or both, one or more mass storage devices for storing information, e.g., magnetic, magneto optical disks, or optical disks. However, a computer need not have such devices. Moreover, a computer can be embedded in another device, e.g., a mobile telephone, a smartphone or a tablet, a touchscreen device or surface, a personal digital assistant (PDA), a mobile audio or video player, a game console, a Global Positioning System (GPS) receiver, or a portable storage device (e.g., a universal serial bus (USB) flash drive), to name just a few.

Computer readable media suitable for storing computer program instructions and information include various forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and (Blue Ray) DVD-ROM disks. The processor and the memory can be supplemented by, or incorporated in, special purpose logic circuitry.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well. In addition, a computer can interact with a user by sending documents to and receiving documents from a device that is used by the user; for example, by sending web pages to a web browser on a user's client device in response to requests received from the web browser.

Implementations of the subject matter described in this specification can be implemented in a computing system that includes a back end component, e.g., as an information server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital information communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, the server can be in the cloud via cloud computing services.

While this specification includes many specific implementation details, these should not be construed as limitations on the scope of any of what may be claimed, but rather as descriptions of features that may be specific to particular implementations. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Particular implementations of the subject matter have been described. Other implementations are within the scope of the following claims. For example, the actions recited in the claims can be performed in a different order and still achieve desirable results. In one embodiment, the processes depicted in the accompanying figures do not necessarily require the particular order shown, or sequential order, to achieve desirable results. In some implementations, multitasking and parallel processing may be advantageous. Accordingly, other aspects, advantages, and modifications are within the scope of the following claims.

1. A computer-implemented method for processing data in one or more data processing devices to determine a likelihood score for an immune-related adverse event associated with an immunotherapy given to a test subject, the method comprising:
inputting, into a classifier, data representing one or more values for a classifier parameter that represents a gene-specific level of mRNA transcribed from a gene of a set of genes in a sample of blood collected from a test subject who was treated with the immunotherapy prior to collecting the sample, with the input data specifying a gene-specific level of mRNA transcribed from each gene of the set of genes in the sample of blood of the test subject, the set of genes comprising CCR3 and PTGS2, with the classifier being for determining a likelihood score indicating whether the gene-specific levels of mRNA transcribed from each gene in the set of genes classifies with (A) a set of immunotherapy-intolerance levels, the set of immunotherapy-intolerance levels being a set of gene-specific levels of mRNA transcribed from each gene of the set of genes in blood samples collected from a first group of individuals who were treated with the immunotherapy prior to collecting the sample, wherein each individual of the first group experienced the immune-related adverse event associated with the immunotherapy; as opposed to classifying with (B) a set of immunotherapy-tolerance levels, the set of immunotherapy-tolerance levels being a set of gene-specific levels of mRNA transcribed from each gene of the set of genes in blood samples collected from a second group of individuals who were treated with the immunotherapy prior to collecting the sample, wherein each individual of the second group did not experience the immune-related adverse event associated with the immunotherapy;
for each of one or more of the genes in the set, binding, by the one or more data processing devices, to the classifier parameter one or more values representing a gene-specific level of transcribed mRNA from that gene as specified by the input data;
applying, by the one or more data processing devices, the classifier to bound values for the parameter;
determining, by the one or more data processing devices based on application of the classifier, the likelihood score for the immune-related adverse event for the test subject; and
outputting, by the one or more data processing devices, information indicative of the determined likelihood score for the immune-related adverse event for the test subject.

2. The computer-implemented method of claim 1,
wherein the input data comprise one or more records that each have one or more values for the parameter representing the level of transcribed mRNA; and
wherein determining the likelihood score for the immune-related adverse event for the test subject comprises:
determining, by the one or more data processing devices based on application of the classifier to the input data comprising the one or more records, the likelihood score for the immune-related adverse event for the test subject.

3. The computer-implemented method of claim 1, wherein the immune-related adverse event is Grade 3 diarrhea, Grade 4 diarrhea, or colitis.

4. The computer-implemented method of claim 3, wherein the second group of individuals did not experience diarrhea, or experienced diarrhea no more severe than Grade 1 or Grade 2 diarrhea.

5. The computer-implemented method of claim 3, wherein the set of genes comprises CCR3, MMP9, and PTGS2.

6. The computer-implemented method of claim 5, wherein the set of genes further comprises at least one gene selected from the group consisting of CARD12, CCND1, IL5, F5 and GYPA.

7. The computer-implemented method of claim 5, wherein the set of genes further comprises CARD12, CCND1, IL5, F5 and GYPA.

8. The computer-implemented method of claim 1, wherein the immune-related adverse event is Grade 2, Grade 3, or Grade 4 diarrhea, or colitis.

9. The computer-implemented method of claim 8, wherein the second group of individuals did not experience diarrhea, or experienced diarrhea no more severe than Grade 1 diarrhea.

10. The computer-implemented method of claim 8, wherein the set of genes comprises CCL3, CCR3, IL8, and PTGS2.

11. The computer-implemented method of claim 10, wherein the set of genes further comprises at least one gene selected from the group consisting of CARD12, F5, MMP9, SOCS3, IL5, and TLR9.

12. The computer-implemented method of claim 10, wherein the set of genes further comprises CARD12, F5, MMP9, SOCS3, IL5, and TLR9.

13. The computer-implemented method of claim 10, wherein the set of genes further comprises at least one gene selected from the group consisting of CARD12, CDC25A, CXCL1, F5, FAM210, GADD45A, IL18BP, IL2RA, IL5, IRAK3, ITGA4, MAPK14, MMP9, SOCS3, TLR9, and UBE2C.

14. The computer-implemented method of claim 10, wherein the set of genes further comprises CARD12, CXCL1, F5, FAM210, GADD45A, IL18BP, IL2RA, IL5, IRAK3, ITGA4, MAPK14, MMP9, SOCS3, TLR9, and UBE2C.

15. The computer-implemented method of claim 1, wherein the classifier has a form:
Y=α+ΣβᵢXᵢ
wherein
Y is a likelihood score indicating a probability that the set of test levels classifies with the set of immunotherapy-intolerance levels, as opposed to the set of immunotherapy-tolerance levels,
Xᵢ is a level of mRNA transcribed from an ith gene of the set of genes in blood of the test subject,
βᵢ is a logistic regression equation coefficient for the ith gene,
α is a logistic regression equation constant that can be zero, and
βᵢ and α are the result of applying logistic regression analysis to the set of immunotherapy-intolerance levels and the set of immunotherapy-tolerance levels.

16. One or more machine-readable hardware storage devices for processing data to determine a likelihood score for an immune-related adverse event associated with an immunotherapy given to a test subject by storing instructions that are executable by one or more data processing devices to perform the computer-implemented method of claim 1.

17.-29. (canceled)

30. A system comprising:
one or more data processing devices; and
one or more machine-readable hardware storage devices for processing data to determine a likelihood score for an immune-related adverse event associated with an immunotherapy given to a test subject by storing instructions that are executable by one or more data processing devices to perform the computer-implemented method of claim 1.

31.-43. (canceled)

44. A method comprising:
a) obtaining a biological sample from a subject who is undergoing immunotherapy;
b) determining, from the biological sample, gene-specific levels of mRNA transcribed from each gene of a set of genes, wherein the set of genes comprises CCR3 and PTGS2,
c) determining that the gene-specific levels of mRNA transcribed from each gene in the set of genes are classified with (A) a set of immunotherapy-intolerance levels, the set of immunotherapy-intolerance levels being a set of gene-specific levels of mRNA transcribed from each gene of the set of genes in blood samples collected from a first group of individuals who were treated with the immunotherapy prior to collecting the sample, wherein each individual of the first group experienced an immune-related adverse event associated with the immunotherapy; rather than being classified with (B) a set of immunotherapy-tolerance levels, the set of immunotherapy-tolerance levels being a set of gene-specific levels of mRNA transcribed from each gene of the set of genes in blood samples collected from a second group of individuals who were treated with the immunotherapy prior to collecting the sample, wherein each individual of the second group did not experience the immune-related adverse event associated with the immunotherapy;
d) either (i) providing to the subject a degree of monitoring for early symptoms related to development of the immune-related adverse event that is heightened compared to the degree of monitoring (if any) for the early symptoms provided to the subject between the start of the immunotherapy and the time the determination of (c) was made; or (ii) administering to the subject a treatment suitable for reducing the likelihood the subject will actually experience the immune-related adverse event; or (iii) reducing the subject's dosage of the immunotherapy; or
(iv) a combination of any two or more of (i), (ii), and (iii).

45. The method of claim 44, wherein the immune-related adverse event is Grade 3 diarrhea, Grade 4 diarrhea, or colitis.

46. The method of claim 45, wherein the second group of individuals did not experience diarrhea, or experienced diarrhea no more severe than Grade 1 or Grade 2 diarrhea.

47. The method of claim 45, wherein the set of genes comprises CCR3, MMP9, and PTGS2.

48. The method of claim 47, wherein the set of genes further comprises at least one gene selected from the group consisting of CARD12, CCND1, IL5, F5 and GYPA.

49. The method of claim 47, wherein the set of genes further comprises CARD12, CCND1, IL5, F5 and GYPA.

50. The method of claim 44, wherein the immune-related adverse event is Grade 2, Grade 3, or Grade 4 diarrhea, or colitis.

51. The method of claim 50, wherein the second group of individuals did not experience diarrhea, or experienced diarrhea no more severe than Grade 1 diarrhea.

52. The method of claim 50, wherein the set of genes comprises CCL3, CCR3, IL8, and PTGS2.

53. The method of claim 52, wherein the set of genes further comprises at least one gene selected from the group consisting of CARD12, F5, MMP9, SOCS3, IL5, and TLR9.

54. The method of claim 52, wherein the set of genes further comprises CARD12, F5, MMP9, SOCS3, IL5, and TLR9.

55. The method of claim 52, wherein the set of genes further comprises at least one gene selected from the group consisting of CARD12, CDC25A, CXCL1, F5, FAM210, GADD45A, IL18BP, IL2RA, IL5, IRAK3, ITGA4, MAPK14, MMP9, SOCS3, TLR9, and UBE2C.

56. The method of claim 52, wherein the set of genes further comprises CARD12, CXCL1, F5, FAM210, GADD45A, IL18BP, IL2RA, IL5, IRAK3, ITGA4, MAPK14, MMP9, SOCS3, TLR9, and UBE2C.

57-62. (canceled)

63. A method comprising:
a) obtaining a biological sample from a subject who is undergoing immunotherapy and is identified as having one or more diarrhea symptoms;
b) determining, from the biological sample, gene-specific levels of mRNA transcribed from each gene of a set of genes, wherein the set of genes comprises CCR3 and PTGS2,
c) determining that the gene-specific levels of mRNA transcribed from each gene in the set of genes are classified with (A) a set of immunotherapy-intolerance levels, the set of immunotherapy-intolerance levels being a set of gene-specific levels of mRNA transcribed from each gene of the set of genes in blood samples collected from a first group of individuals who were treated with the immunotherapy prior to collecting the sample, wherein each individual of the first group experienced Grade 2, Grade 3, or Grade 4 diarrhea at some point during that individual's immunotherapy treatment period; rather than being classified with (B) a set of immunotherapy-tolerance levels, the set of immunotherapy-tolerance levels being a set of gene-specific levels of mRNA transcribed from each gene of the set of genes in blood samples collected from a second group of individuals who were treated with the immunotherapy prior to collecting the sample, wherein each individual of the second group experienced Grade 1 diarrhea at some point during that individual's immunotherapy treatment period but did not experience a higher grade of diarrhea during that period; and
d) administering an anti-inflammatory agent to the subject.

64. The method of claim 63, wherein the set of genes comprises CCL3, CCR3, IL8, and PTGS2.

65. The method of claim 64, wherein the set of genes further comprises at least one gene selected from the group consisting of CARD12, F5, MMP9, SOCS3, IL5, and TLR9.

66. The method of claim 64, wherein the set of genes further comprises CARD12, F5, MMP9, SOCS3, IL5, and TLR9.

67. The method of claim 64, wherein the set of genes further comprises at least one gene selected from the group consisting of CARD12, CDC25A, CXCL1, F5, FAM210, GADD45A, IL18BP, IL2RA, IL5, IRAK3, ITGA4, MAPK14, MMP9, SOCS3, TLR9, and UBE2C.

68. The method of claim 64, wherein the set of genes further comprises CARD12, CXCL1, F5, FAM210, GADD45A, IL18BP, IL2RA, IL5, IRAK3, ITGA4, MAPK14, MMP9, SOCS3, TLR9, and UBE2C.
